SEQUENCE LISTING (17 sequences)

SEQ ID NO: 1 - DNA, 16357 bp, Homo Sapiens
ctgagcgccgcgggcctgcgccattgaggagcggcggggaggaaacgccgcgcagcgccgggctggggcgggcggcccgggacaccgacagatttttctgtgaccatgaaagagagaaataaagaatgatccatgatttctaaacaccttttcctgaggatatagtcatgttggaaggccttgtagcctgggttctcaatacctatttgggaaaatatgtcaataacctgaacactgaccagctctcagttgcacttctcaaaggtgctgttgaattagaaaacttgccattaaagaaagatgccttgaaagaattggaattaccatttgaagtcaaagctggcttcattgggaaagtaacccttcagattcccttttatcgcccccatgtggacccttgggtgatctccatctccagccttcacttaattggagccccagagaaaatacaggatttcaatgatgaaaaggagaagctgttggaaagggaacgtaagaaagcactacttcaagccctggaggagaaatggaagaatgaccgccagcagaaaggggagtcctattggtattcagttaccgcctccgtagttacaaggattgtggagaatattgaattaaaaattcaagatgtccatttacgctttgaagatggtgtcaccaatccctcccatccttttgcttttggcatctgcattaagaatgtgtccatgcaaaatgctgtgaatgagcctgtacagaaactaatgcggaaaaagcaattagacgtagcagaatttagcatctattgggatgtcgattgcactttactgggggatttgcctcagatggagttacaggaggccatggccaggagcatggagagtcgcagccatcactacgtcctggagcctgtgtttgcatctgctcttttgaagagaaactgctccaagaagcccctgcggtctcggcacagtccccgtattgattgtgatattcagctggagaccattcccttgaaactctctcagctgcaataccggcaaatcatggaattcctcaaggagctggaacgaaaggagaggcaggtgaagttccgaaggtggaaacccaaggtggcgatatctaagaactgccgagaatggtggtattttgctttgaatgctaacttgtatgagatcagagagcagaggaaacgttgcacctgggactttatgttgcaccgcgctcgtgatgctgtatcttacactgacaaatatttcaacaagttaaaaggaggcctgctgtccacagatgacaaggaggaaatgtgtcggattgaagaggaacagagctttgaggaattgaagattttgcgtgaactggttcatgatcgatttcacaaacaggaagaactagcagagagtctgcgggagcctcagtttgattctccaggagcctgtccgggagccccagaacccggtggaggcagtgggatgctgcagtatctccagtcctggtttcctggatggggtggctggtacgggcagcagaccccagaagggaatgtggttgagggactgtcagcagagcaacaggagcagtggattcctgaagagatcctgggcactgaggagttttttgaccccactgcagatgcctcgtgtatgaacacgtatacaaagcgagatcatgtctttgccaaactgaatttgcagttgcagcgaggtacagtgactctgttacacaaggagcaaggaactcctcaaatgaatgaaagtgctttcatgcagctcgagttttcagatgtaaaacttctagcagagtctcttcctcgaagaaattcctcgttgctttcagtccggttgggtggactgtttcttcgagacctggctacagaaggaactatgtttcctcttctagtcttccctaatccacaaaaagaagttggcagagtctcacaatcttttggtctacaaactacatctgcagacagaagtgatcattacccagctgcagatccagatggccccgtttttgagatgctgtatgagagaaatccggcgcacagccactttgagaggcggctcaatgtcagcacaaggcccttgaacatcatatacaatccgcaggccattaaaaaagtagcagactttttctacaagggaaaggttcatacctcaggttttggttatcagtctgaacttgagctgagagtggctgaagctgcccgaagacaatataacaagctgaagatgcagaccaaggcagaaatccggcaaactcttgatcgtttgctagtgggtgatttcattgaggagagtaaacgatggaccgtgcggctggatatttctgcccctcaggtgatatttcctgatgatttcaaattcaagaatcctgtgttagttgtcgtggatctaggaagaatgcttttgacgaacacccaagataactccaggaggaaaagtagggatgggtcagcatctgaagagacccagtttagtgatgatgaatataagacccccctggccacacctcctaacaccccacctcccgagtcaagcagcagcaacggagagaaaacacctcccttttctggagttgagttcagtgaagaacagcttcaagcacatttaatgagcacaaagatgtatgagaggtactcgctgtcatttatggacctccagatcatggttggacgagtgaaagacaattggaagcatgtccaggatattgacgtgggaccaacacatgtggtagagaagttcaacgttcacctacagttagagcgtcgattgatttatacttcagatcccaaatatccaggagccgtgctctcaggcaacttaccagacttaaaaatccacattaatgaagataaaatatctgcactaaagaattgctttgctctcctcaccaccccagaaatgaaaacttctgacactcagattaaagaaaagatttttccccaggaggagcagcggggaagtttgcaagactccgtaatgaatttaacccagagcattgtgttgttggagcagcatacccgcgaggttctggtggagtcgcagctcctcctggcggaatttaaagtgaactgtatgcagcttggtgttgagagcaatggccggtacatttctgtgctcaaggtgtttggtaccaatgctcactttgtgaagaggccttatgatgctgaagtctccctaactgttcatggtttgctcctggtggataccatgcagacatatggtgctgattttgaccttttgatggcttcacataagaacttgagctttgatattccaacgggaagccttcgggatagcagggcccagtctcctgtctctggaccgaatgtggcccacttaactgatggagctacactgaacgaccgatcagctactagtgtttcacttgacaaaattcttaccaaagagcaagagtcccttattaagttggaatatcagtttgtgagttcagagtgcccatcgatgaatttagacagtactcttcaggtgatttccctacaggtgaataatttagatattatcctcaatccagagacgattgtggagctaattggttttcttcaaaaatcctttcccaaggaaaaagatgatttaagtcctcaacctttaatgactgattttgaaagaagcttcagagaacaaggaacttaccagtctacatatgaacaaaacactgaggttgcagtggaaatccataggctgaacttactgcttcttcggacagtgggcatggcaaatagagagaaatatggcagaaaaattgcaactgcaagtataggtggcaccaaagttaatgtctcaatgggtagcacgtttgacatgaatggttctcttggctgtttacagcttatggatttgacacaagataacgttaaaaaccagtatgttgtcagcattgggaattctgtaggctatgaaaatatcatcagtgatattggctactttgaatctgtgtttgtcagaatggaagatgcagccctcactgaagctttgagtttcacgtttgttgagagatctaaacaggagtgttttctcaacctgaagatggcttctttacattataaccactctgctaagtttttgaaggagttgacgttatccatggatgaactggaagaaaattttcgaggtatgctgaaaagcgcagccaccaaagtcaccacagtactagctaccaagactgccgagtatagcgagatggtatcgctctttgaaactccaaggaagactcgggaaccctttatcttagaggaaaatgaaatatatgggtttgacctagcttcgtctcatttggacactgtaaagctaatcttgaacataaacattgaatcaccagttgtttctatccctcggaagccggggagtcctgagttgttggtgggacacttgggacagatattcatccagaattttgtggcgggagatgatgaatccagaagtgaccgtctgcaggtggaaatcaaggacattaaactgtattctttgaattgcacccagttggcaggtagagaagctgttgggtctgaaggaagccggatgttttgcccaccttccgggtctggcagtgccaacagtcaggaggaagctcatttcacacgacatgatttctttgaatctttgcatagaggtcaagcttttcacatcctgaacaacaccaccattcagtttaaactggagaagatccctatagagagagaatctgaattgactttttctcttagcccagatgacctgggaacttctagcatcatgaagattgaaggaaaatttgtcaatccagttcaggtggtgttagcaaagcatgtatatgagcaggttttacaaaccctggacaatctcgtgtacagtgaagatctgaataagtatccagccagtgctacctcctccccttgccctgattctcctctgcctcccctcagtacctgtggagaatcttctgttgaaaggaaggagaatggattgttcagccactccagcctttctaacacctctcagaagtcattgtcagtgaaggaagtcaaatcctttactcagattcaagccaccttttgtatatcagagcttcaggttcagctaagtggagatctgactttgggggcccaaggtcttgtgagcttaaagtttcaggactttgaggtggaattcagtaaagaccatccccagactttatctattcagattgccctgcattctctgctgatggaggacttattggagaagaatccagattctaaatataagaacctgatggtgtctcgaggagcccctaagccatctagtttagcacaaaaagaatacctttctcagtcttgcccctcagtgtccaatgtggaatatcctgatatgcctcggtctctcccttcccacatggaagaagctcctaatgtcttccagttgtatcaaaggcccacctctgcgtcccggaaaaagcaaaaggaagtccaagacaaggactatcccttgaccccacctccttctccaacagtggatgagcccaagatacttgttggaaagagtaaatttgatgattccttagtccacatcaacatattcttggtagataagaaacatccagaattctcttccagttacaatcgagttaaccggagcattgatgttgattttaattgcttggatgtgctgatcacactgcaaacctgggttgtgatattagacttttttggaatcggctccactgcagacaaccacgcaatgaggctgcctcctgagggcattctgcacaacgtgaagttggagccacatgcctccatggagtctggacttcaggatccagtgaacaccaaactggatctcaaggttcattcactttctctagtgctgaataagaccaccagtgagcttgccaaagcaaatgtgtccaaattagtagcacacctggaaatgattgagggagacctggccttacagggcagcattgggagtctgtctctaagtgacctcacatgccatggagagttctacagagaacggttcactaccagtggtgaagaagcactcatcttccagacttttaaatacggacggcctgaccctctgctccggagagaacacgacattcgcgtgagcctccggatggcctctgtgcagtatgtgcatactcagcgtttccaggcagaggtggtggccttcattcagcatttcactcagctgcaggatgtcttagggcgccagcgagctgctattgaggggcagacggtgagagatcaagcccagcgctgttcacgggttctcctggatattgaggctggtgctcccgttctcttgatcccagaaagttccagatcaaataatctgattgtagcaaatttggggaagttgaaagtcaaaaataagtttctgtttgctggttttcctggcaccttttccctacaagataaggaatctgtgccttcagcttccccaacgggtattcccaaacacagtctgaggaaaacgacaagcacggaggagcccaggggaacccattcccaggggcagttcacgatgcctcttgctggaatgagcctaggaagcctgaagagtgagtttgtgcccagtacctccaccaagcagcaagggccgcaacccacactgtctgttggccaagagtccagtagtccagaagaccatgtctgcctgctggattgcgttgtcgtggatctccaggacatggacatctttgctgcagagagacatccgagagaatactcgaaggcaccagaggatagtagtggagatctgatcttcccttcctattttgtgcgacagacaggaggaagcctcttaaccgagccttgtaggctgaaattgcaggtggaaaggaatttggacaaagaaataagtcatactgtgccagacatatctatccatggcaatctctcctcagtccactgctctctggatctgtataaatacaagctgatccgcggcttattagagaacaacctgggagaacccatagaggaatttatgcggccttatgatttacaagatccaagaattcatactgtcctgagtggagaagtgtacacctgtatgtgcttcctcattgatatggtgaatgtaagtctggagcttaaagatccaaaaagaaaagaaggtgctgggtccctagccagatttgacttcaagaaatgcaaactgctctatgaaagtttttccaaccaaaccaagtccattaacttggtttcccattccatgatggcttttgacacccgttatgctgggcagaagaccagccctggcatgacgaatgtgttcagctgtatctttcagcccgctaagaacagcagcaccacccaagggtccattcagattgaactacatttcagatctaccaaggattcctcctgctttacagtagttctcaacaatctccgtgtgtttctcatatttgactggctactgttagtccatgattttctccacactcccagtgatattaagaaacaaaatcatgttactccttctcgccaccgtaactctagcagcgaatctgctatagttcccaaaactgtgaagagtggagtagttaccaagcggtcttcccttcctgtgtccaatgaaaggcacctggaggtcaaggtcaatgtaacaggtacggagtttgtggtcattgaagatgtgtcctgcttcgacaccaatgccattattctgaaaggcaccacagtgctcacctataagccccggtttgttgatcgccccttttcaggaagtttgtttggcattgaggtgttttcatgccgactagggaatgagcatgatacagctctttcaattgtggatcccgtacaaattcaaatggagttggtggggaattcttcttatcaaaatagttcaggattgatggatgcattcaatagtgaagatttcccacctgtcctggagattcagttacaagccctggatatcagactctcctataatgatgttcagctgtttcttgccattgcaaaatccatcccagagcaagctaatgctgcagtgccagactcagtggccctggagtcagactccgttggcacttaccttccaggtgcatctcgcgttggagaggaaatcagagaagggacaagacacaccttagatcctgtcttggagttacagctggctaggctgcaggagctgggattcagcatggatgattgtcgcaaagctcttttggcgtgtcaaggccaattgaaaaaggcagcaagttggttgtttaagaatgcggaacctctgaagtctctttccttggcctccaccagccgagatagcccaggggctgtggcagcgccattgatctctggcgtggagatcaaagctgagagtgtgtgcatctgtttcatcgatgactgcatggattgtgatgttcctctcgctgaactcaccttttcccgtctgaattttcttcagcgtgtaagaactagccctgaaggctatgcccacttcaccctttctggagattattataaccgtgctctttcaggctgggagccatttattgagccttggccatgctctgtatcctggcaacagcaggcagctagtcgtctccatcctcctcgactgaagctagaagccaaggccaaacctcgtttggatatcaatatcacttctgtgctaattgaccagtatgtaagtaccaaggaatcgtggatggcagattactgtaaagatgacaaggacatagagtcagctaaatcagaagactggatgggctcttcggtggatcctccatgttttggacaaagcctcccccttgtctaccttagaactaggagtacagccagtctgactaacctagagcaccagatctatgctagagcagaggtgaaaacccccaagcgccggcagccatttgtcccctttgctctgaggaaccacacggggtgcactttgtggtttgccaccctgaccaccacacccaccagagctgcactctctcacagtgggagtccaggggtagttccagaagggaacggaacatttctcgatgatactcacaatgttagtgaatggcgagaagtccttacaggtgaagagattccctttgaatttgaagcaagaggaaagttaagacacagacacacccatgacctccggattcatcaactgcaagtgagagtaaatggctgggagcaagtgagcccagtgtctgtggacaaagtcgggaccttttttcgatatgcagcaccagataaaaattcatcttcctctacgattggcagcccaagcagcagaacaaatattatacatccccaggtttatttctcttcactcccaccagtgcgggtggtctttgcagtgactatggaaggcagtgcacggaaagtcatcactgtccggtcagccctcattgtgaggaacagacttgagacaccaatggaactaagactggatagcccatcagctccagacaagccagtggtgcttcctgctatcatgccaggggattcgtttgctgtgcctttacacctcacttcttggcggctacaggcccggcccaaaggattgggtgtatttttctgtaaggctcccattcattggaccaatgtagtgaagactgcagaaattagtagcagtaaacgagagtgccactctatggacacagaaaaaagccgatttttcaggttttgtgtggctataaagaaagagaattatccagattatatgccctcaaacatattttctgacagtgcaaaacagattttcagacagcctgggcataccatatatctcctgccaactgtggtaatctgcaacttgctaccctgtgaacttgatttttatgttaaaggaatgccaattaatgggacgctgaaacctggcaaggaggcagctctccatacagctgatacatcccagaacattgagctgggggtatcactggagaatttccccctctgtaaagaattgctcattccacctggaacccaaaactatatggtgagaatgcgactctatgacgtcaaccgtcggcagctgaacctcaccatccggattgtgtgtcgagcagaaggatccttaaagatcttcatttctgctccatattggctgattaacaaaacagggttgccactgatcttcagacaggacaatgccaagacagatgctgcaggccagtttgaggagcatgagctggcccgtagcctgagtcctctcttattctgctatgctgacaaagagcagccaaacctctgcacgatgagaatcggaagggggattcatccagaaggcatgccgggctggtgtcagggcttctccctggatggtggtagtggtgtccgagctttgaaagtcatccagcaaggaaaccgcccagggctgatctataacattggtattgatgtcaagaaaggccgaggtcgatacattgatacctgcatggtcatctttgccccccgttacctgttagataataaatcatctcacaagcttgcatttgcacagagggaatttgccaggggacagggaacagccaatcccgaaggttacatttccacccttcctggttccagtgtggtgttccactggcctcggaatgactatgatcagctattgtgtgtcagactgatggacgttcccaattgtatttggtctggaggctttgaagtcaacaagaataattccttccatatcaacatgagggataccttgggaaaatgcttcttcctacgagtggaaattactctccgaggagctacgtataggatctcatttagtgacacagatcagttacctcctcctttccgaattgacaacttttctaaggtcccggttgtctttactcagcatggcgtagctgaacccaggctccggactgaagtgaagcccatgacttcattggattatgcctgggacgaacccaccttgccaccttttatcactctgactgttaaaggggcagggtcctctgagatcaactgcaacatgaatgatttccaggataatcggcagctttattatgaaaatttcatttacattgctgctacatatacattctctggcttgcaggagggaacaggcaggcctgtggcttccaacaaggccattacctgtgcggagctcgttttggatgtctcacccaagacacaaagagtcattttaaaaaagaaggaaccaggaaagcgttctcagctgtggaggatgacaggaacaggaatgctggcccatgagggctcctcagttcctcacaatcccaataagccctcagccgcccgctccaccgaggggtctgccatcttagatattgctggtctcgctgcagtgactgacaacagatacgagccactgatgctgagaaagcctgaccgcaggcgaagcacaactcagacgtggagtttccgagaaggaaaactgacctgtgggttacatgggttggtcgtccaggccaaaggaggactttctggtttgtttgatggagctgaagttgttcttggtcctgacacttccatggagcttttggggccagttccacctgaacaacaatttattaatcaaaaaatgagacctggttctggaatgttatccatcagagtcatcccagatggaccaactagagcactccagataacagatttctgccaccggaaaagcagccgttcatatgaagtggatgaacttcctgtcaccgaacaagagctgcagaaattaaagaatccagatacagagcaggaattggaagtgcttgtgaggttagaaggtggaattgggttgtccttaattaataaagtcccagaagaactggtctttgcaagtcttacaggaatcaatgtgcactatacacagctggcaaccagtcacatgcttgaactcagcatacaggatgtacaggtggacaatcagctcattggtaccacgcagcccttcatgctctatgtgactcccctgagcaatgagaatgaggtcatcgagaccggcccagctgtgcaagtcaacgcagtgaagttccccagtaagagtgcactgaccaacatctacaagcatctgatgatcacagctcagagattcacagtgcaaattgaggagaaactgctcctcaagctgctaagtttctttggctacgatcaagcagaatcagaggtggaaaaatatgatgaaaacctccatgaaaagacagctgagcaaggtggaacaccaattcgatactactttgaaaatctcaaaatcagcattcctcagatcaagctaagtgtgttcacctccaacaagctcccattggatcttaaggccctaaaaagcaccttggggtttcctttgatacggtttgaagacgctgtgattaatctagatccattcactcgggtacatccctatgagaccaaggagttcatcatcaatgatatcctcaaacatttccaggaggaactcctcagccaggcagctcgaatcctgggatcagtggattttcttggcaatcctatggggcttttgaatgatgtttctgaaggggttactggactgataaaatatggaaatgtcgggggcctcatcagaaatgttacacacggagtatcaaactctgctgccaagtttgctggaacattatcagatggcttagggaagacgatggacaatcggcatcagtcagagcgggagtacatcaggtaccatgcagccacaagtggtgaacaccttgtagccggcatccatggcctggctcatggtatcattggtggactgaccagtgttataacttcgacagtggaaggtgtgaaaacagaagggggtgtcagcggtttcatatctggccttggaaaagggcttgttggcactgtaaccaagccagtggcaggcgccctggattttgcatcagaaacagcccaggcggtgagagacacagccacactcagcggccccaggactcaagcacagagggttcggaaaccgcgttgctgcacggggccccaggggctgcttccccgatattctgagagccaggcggaaggacaggagcagctcttcaaactcacagacaacatacaggacgaattcttcatcgctgtggagaacattgacagctactgcgtgctcatctcctccaaagctgtttacttcctgaaaagtggagactacgtggatcgagaagccattttcctagaagtcaaatacgatgacctctaccactgccttgtctccaaagaccatgggaaggtgtatgtgcaggtgaccaagaaagccgtgagcacgagcagtggagtgtccatccccggcccctcccaccagaagcccatggtccatgtgaaatctgaggtccttgctgtcaagttgtcacaagaaataaactacgcaaagagcctctactatgaacagcagcttatgttaagactcagcgaaaaccgagagcagctggagctggactcctgaagccccgctgctgagatgggcgctcccgacacagcgcagacccaccaggaggaaagaggcccagctctcagctgacgatggaggcagaaccggagtcgggtttggggaagttgtcaaggaatgagggaaagtaaatcctcatgaggaaaagtacaaatggaaatcgtattaatttgtgaggcagggagttattttagattatgggaaataatttttaaaggtattggttaaataacgtttaaaaacatgtactgagatgaatctaatttttagattgccctgtattttgttaacatgtatatatgtacaacagtgtgtttgtaaatatataggaacgtttctgaacagggtctgtgctatgtgtaaaggtttgttaactgtaaagtaatataaagttatattggatcttctattgcactaattctagatgtctaattcaggatactgtctatagaaaggcattcttaaaagttaaagaatgttacgtcttagttttggagactaaagtattcccagtaaagtgggttgaggtgagggctgtggtcctgaaagggacgcctttgacatcgtggctgtccagttgggctgtgagctgtggcacccaggactggcgctggcccttcagaaggatctaggagaggggcttgggagcccacttttaatttctcacccccattttacaaagagtgcttagattcttacaaattatgatgtaagttatccatttggctttttcctaactagtcttaccaaacttagggggaaacctgtgctccattaccacatgggtgcaagtcagcattgtaagttttctcaggttattattattagagaggttggaaacattggtaaactctgttgattgagaaggaaaaaaaaagtcccattgaactgttgcaacaaatcagaaatccacataaaagtgctctcctgcctgggcagcaacaaccaagaacaaagccccgggactgttttctttttaataaagccacaggcaggcatcgtagctccacagcccgaggggacacaggatggaaaccccaggatgagaagggagcagggagagttccagaaagggggatgaaataggagtattaaaaagctgcgttggtaagtttttcatggaaccaagatttgacaaaggcatctcttatccttggttttaaattcctgctgggagcaaggcctggtatgagcgccctgggtcttgtttttggtgtttcgcttttctgtaaggattaagcagatagggagaagggaaaaggggcctcactttagaatgaatgagtcaccttgtgatttttaaatttttattttaataaagctaatcaatttctacaaccttgtcacatgtagctgagtctgggatgactcagtggatcagtggatgcgtggaaggttttggtgtttataactcatgacccaaatccttccaagacacaaatgaagctaatcttcttcccctccctttcctctcccacttcccctctgtgtctgtgtctgtgtgtgtgtgtgtgtgtgtgtgtgtgtgcgcgcgcgcgcgtgcatgcagagaggaaggaagggagctttagcacaagtagctacatagtgttcaaggatcagctttcttgaactccgaggcttggttgcctttgctgaactccaagttagtggattgcagaatggaaacttggcttttgcggcactgggtgagttttagtttgtgtgtgtcttgctggggggtggtgatgattgtctcagcactcacgcactgcacaagatggcagcaggatacagcactgcacaagatggcagctcctctgcagcttcctcctcagcctccctccttgcacccccacaggtttggcttgtggtttttgtcatcagtaacctactgcctgagatcatgatctcttaaaagatgagactctcggaagggttgattgtatgcgtcagtgagccttctatcaccttctggaacaaagtcacttgaaatctcttgatgagattaaggagtttagtgttactaagaaaatctgctttgggccgcagcagtgctgggtgttctcagacctgactgaggaagttagctgcgggctgccctgtgggctggtgcttcaggaggaatccagagaagtgttcagatgccccccttgggctcctttctaattttaatcagctctttaaatagctgcccatctcctgtgattgcacaaccaagcactttgacatttgcaccttaggagaggcagatgttaaaatggaatccaaagaccacctagggcggggctgggtgggagatgggagggccaactgcgagctgctccacttctcagctctcccctgccctgcagccctgggccagacaaggccagaaggtttcaggggcatttgacatcccctcctggttctcaccaggaaaacatccaaagctttggaggaaacaggccctgcccctggctccttaaatgccccgtctctttgtaaactgatattcagccagcaatgcctaagactttgttaagatcatttctactgcttttctttctgcttcaaacacacagttcgtctctgaggaaagtaaaataaatggaataagagtaaattgggtaaggagatatccaaagctacccagtcccttgacccagcacagttggccgacccgtgtcactccctggctgtcgctgcttctctgtgctcactgaagggtgagccaggccagtgcttccccagcccctgggcctggtcactacacagtggaaaacagacaagcggccccttccccaaatcccaagagtgtcttgctgcttggtgggtgctcatcgcaatgttctgaaggctccagggccactttgtttgtaagtatgatctgggcctcaaaataccatagtagctgcttgataaaattctaaaaatatctggttctctattatgtaaacactattacagtcaccagtgtgtgaagactcttgagtctggttctcatatcagagtcatcatttttcttcctgtggaataaaatgccttgtggacttcccaaa

SEQ ID NO: 2 - DNA, 16282 bp, Homo Sapiens
ctgagcgccgcgggcctgcgccattgaggagcggcggggaggaaacgccgcgcagcgccgggctggggcgggcggcccgggacaccgacagatttttctgtgaccatgaaagagagaaataaagaatgatccatgatttctaaacaccttttcctgaggatatagtcatgttggaaggccttgtagcctgggttctcaatacctatttgggaaaatatgtcaataacctgaacactgaccagctctcagttgcacttctcaaaggtgctgttgaattagaaaacttgccattaaagaaagatgccttgaaagaattggaattaccatttgaagtcaaagctggcttcattgggaaagtaacccttcagattcccttttatcgcccccatgtggacccttgggtgatctccatctccagccttcacttaattggagccccagagaaaatacaggatttcaatgatgaaaaggagaagctgttggaaagggaacgtaagaaagcactacttcaagccctggaggagaaatggaagaatgaccgccagcagaaaggggagtcctattggtattcagttaccgcctccgtagttacaaggattgtggagaatattgaattaaaaattcaagatgtccatttacgctttgaagatggtgtcaccaatccctcccatccttttgcttttggcatctgcattaagaatgtgtccatgcaaaatgctgtgaatgagcctgtacagaaactaatgcggaaaaagcaattagacgtagcagaatttagcatctattgggatgtcgattgcactttactgggggatttgcctcagatggagttacaggaggccatggccaggagcatggagagtcgcagccatcactacgtcctggagcctgtgtttgcatctgctcttttgaagagaaactgctccaagaagcccctgcggtctcggcacagtccccgtattgattgtgatattcagctggagaccattcccttgaaactctctcagctgcaataccggcaaatcatggaattcctcaaggagctggaacgaaaggagaggcaggtgaagttccgaaggtggaaacccaaggtggcgatatctaagaactgccgagaatggtggtattttgctttgaatgctaacttgtatgagatcagagagcagaggaaacgttgcacctgggactttatgttgcaccgcgctcgtgatgctgtatcttacactgacaaatatttcaacaagttaaaaggaggcctgctgtccacagatgacaaggaggaaatgtgtcggattgaagaggaacagagctttgaggaattgaagattttgcgtgaactggttcatgatcgatttcacaaacaggaagaactagcagagagtctgcgggagcctcagtttgattctccaggagcctgtccgggagccccagaacccggtggaggcagtgggatgctgcagtatctccagtcctggtttcctggatggggtggctggtacgggcagcagaccccagaagggaatgtggttgagggactgtcagcagagcaacaggagcagtggattcctgaagagatcctgggcactgaggagttttttgaccccactgcagatgcctcgtgtatgaacacgtatacaaagcgagatcatgtctttgccaaactgaatttgcagttgcagcgaggtacagtgactctgttacacaaggagcaaggaactcctcaaatgaatgaaagtgctttcatgcagctcgagttttcagatgtaaaacttctagcagagtctcttcctcgaagaaattcctcgttgctttcagtccggttgggtggactgtttcttcgagacctggctacagaaggaactatgtttcctcttctagtcttccctaatccacaaaaagaagttggcagagtctcacaatcttttggtctacaaactacatctgcagacagaagtgatcattacccagctgcagatccagatggccccgtttttgagatgctgtatgagagaaatccggcgcacagccactttgagaggcggctcaatgtcagcacaaggcccttgaacatcatatacaatccgcaggccattaaaaaagtagcagactttttctacaagggaaaggttcatacctcaggttttggttatcagtctgaacttgagctgagagtggctgaagctgcccgaagacaatataacaagctgaagatgcagaccaaggcagaaatccggcaaactcttgatcgtttgctagtgggtgatttcattgaggagagtaaacgatggaccgtgcggctggatatttctgcccctcaggtgatatttcctgatgatttcaaattcaagaatcctgtgttagttgtcgtggatctaggaagaatgcttttgacgaacacccaagataactccaggaggaaaagtagggatgggtcagcatctgaagagacccagtttagtgatgatgaatataagacccccctggccacacctcctaacaccccacctcccgagtcaagcagcagcaacggagagaaaacacctcccttttctggagttgagttcagtgaagaacagcttcaagcacatttaatgagcacaaagatgtatgagaggtactcgctgtcatttatggacctccagatcatggttggacgagtgaaagacaattggaagcatgtccaggatattgacgtgggaccaacacatgtggtagagaagttcaacgttcacctacagttagagcgtcgattgatttatacttcagatcccaaatatccaggagccgtgctctcaggcaacttaccagacttaaaaatccacattaatgaagataaaatatctgcactaaagaattgctttgctctcctcaccaccccagaaatgaaaacttctgacactcagattaaagaaaagatttttccccaggaggagcagcggggaagtttgcaagactccgtaatgaatttaacccagagcattgtgttgttggagcagcatacccgcgaggttctggtggagtcgcagctcctcctggcggaatttaaagtgaactgtatgcagcttggtgttgagagcaatggccggtacatttctgtgctcaaggtgtttggtaccaatgctcactttgtgaagaggccttatgatgctgaagtctccctaactgttcatggtttgctcctggtggataccatgcagacatatggtgctgattttgaccttttgatggcttcacataagaacttgagctttgatattccaacgggaagccttcgggatagcagggcccagtctcctgtctctggaccgaatgtggcccacttaactgatggagctacactgaacgaccgatcagctactagtgtttcacttgacaaaattcttaccaaagagcaagagtcccttattaagttggaatatcagtttgtgagttcagagtgcccatcgatgaatttagacagtactcttcaggtgatttccctacaggtgaataatttagatattatcctcaatccagagacgattgtggagctaattggttttcttcaaaaatcctttcccaaggaaaaagatgatttaagtcctcaacctttaatgactgattttgaaagaagcttcagagaacaaggaacttaccagtctacatatgaacaaaacactgaggttgcagtggaaatccataggctgaacttactgcttcttcggacagtgggcatggcaaatagagagaaatatggcagaaaaattgcaactgcaagtataggtggcaccaaagttaatgtctcaatgggtagcacgtttgacatgaatggttctcttggctgtttacagcttatggatttgacacaagataacgttaaaaaccagtatgttgtcagcattgggaattctgtaggctatgaaaatatcatcagtgatattggctactttgaatctgtgtttgtcagaatggaagatgcagccctcactgaagctttgagtttcacgtttgttgagagatctaaacaggagtgttttctcaacctgaagatggcttctttacattataaccactctgctaagtttttgaaggagttgacgttatccatggatgaactggaagaaaattttcgaggtatgctgaaaagcgcagccaccaaagtcaccacagtactagctaccaagactgccgagtatagcgagatggtatcgctctttgaaactccaaggaagactcgggaaccctttatcttagaggaaaatgaaatatatgggtttgacctagcttcgtctcatttggacactgtaaagctaatcttgaacataaacattgaatcaccagttgtttctatccctcggaagccggggagtcctgagttgttggtgggacacttgggacagatattcatccagaattttgtggcgggagatgatgaatccagaagtgaccgtctgcaggtggaaatcaaggacattaaactgtattctttgaattgcacccagttggcaggtagagaagctgttgggtctgaaggaagccggatgttttgcccaccttccgggtctggcagtgccaacagtcaggaggaagctcatttcacacgacatgatttctttgaatctttgcatagaggtcaagcttttcacatcctgaacaacaccaccattcagtttaaactggagaagatccctatagagagagaatctgaattgactttttctcttagcccagatgacctgggaacttctagcatcatgaagattgaaggaaaatttgtcaatccagttcaggtggtgttagcaaagcatgtatatgagcaggttttacaaaccctggacaatctcgtgtacagtgaagatctgaataagtatccagccagtgctacctcctccccttgccctgattctcctctgcctcccctcagtacctgtggagaatcttctgttgaaaggaaggagaatggattgttcagccactccagcctttctaacacctctcagaagtcattgtcagtgaaggaagtcaaatcctttactcagattcaagccaccttttgtatatcagagcttcaggttcagctaagtggagatctgactttgggggcccaaggtcttgtgagcttaaagtttcaggactttgaggtggaattcagtaaagaccatccccagactttatctattcagattgccctgcattctctgctgatggaggacttattggagaagaatccagattctaaatataagaacctgatggtgtctcgaggagcccctaagccatctagtttagcacaaaaagaatacctttctcagtcttgcccctcagtgtccaatgtggaatatcctgatatgcctcggtctctcccttcccacatggaagaagctcctaatgtcttccagttgtatcaaaggcccacctctgcgtcccggaaaaagcaaaaggaagtccaagacaaggactatcccttgaccccacctccttctccaacagtggatgagcccaagatacttgttggaaagagtaaatttgatgattccttagtccacatcaacatattcttggtagataagaaacatccagaattctcttccagttacaatcgagttaaccggagcattgatgttgattttaattgcttggatgtgctgatcacactgcaaacctgggttgtgatattagacttttttggaatcggctccactgcagacaaccacgcaatgaggctgcctcctgagggcattctgcacaacgtgaagttggagccacatgcctccatggagtctggacttcaggatccagtgaacaccaaactggatctcaaggttcattcactttctctagtgctgaataagaccaccagtgagcttgccaaagcaaatgtgtccaaattagtagcacacctggaaatgattgagggagacctggccttacagggcagcattgggagtctgtctctaagtgacctcacatgccatggagagttctacagagaacggttcactaccagtggtgaagaagcactcatcttccagacttttaaatacggacggcctgaccctctgctccggagagaacacgacattcgcgtgagcctccggatggcctctgtgcagtatgtgcatactcagcgtttccaggcagaggtggtggccttcattcagcatttcactcagctgcaggatgtcttagggcgccagcgagctgctattgaggggcagacggtgagagatcaagcccagcgctgttcacgggttctcctggatattgaggctggtgctcccgttctcttgatcccagaaagttccagatcaaataatctgattgtagcaaatttggggaagttgaaagtcaaaaataagtttctgtttgctggttttcctggcaccttttccctacaagataaggaatctgtgccttcagcttccccaacgggtattcccaaacacagtctgaggaaaacgacaagcacggaggagcccaggggaacccattcccaggggcagttcacgatgcctcttgctggaatgagcctaggaagcctgaagagtgagtttgtgcccagtacctccaccaagcagcaagggccgcaacccacactgtctgttggccaagagtccagtagtccagaagaccatgtctgcctgctggattgcgttgtcgtggatctccaggacatggacatctttgctgcagagagacatccgagagaatactcgaaggcaccagaggatagtagtggagatctgatcttcccttcctattttgtgcgacagacaggaggaagcctcttaaccgagccttgtaggctgaaattgcaggtggaaaggaatttggacaaagaaataagtcatactgtgccagacatatctatccatggcaatctctcctcagtccactgctctctggatctgtataaatacaagctgatccgcggcttattagagaacaacctgggagaacccatagaggaatttatgcggccttatgatttacaagatccaagaattcatactgtcctgagtggagaagtgtacacctgtatgtgcttcctcattgatatggtgaatgtaagtctggagcttaaagatccaaaaagaaaagaaggtgctgggtccctagccagatttgacttcaagaaatgcaaactgctctatgaaagtttttccaaccaaaccaagtccattaacttggtttcccattccatgatggcttttgacacccgttatgctgggcagaagaccagccctggcatgacgaatgtgttcagctgtatctttcagcccgctaagaacagcagcaccacccaagggtccattcagattgaactacatttcagatctaccaaggattcctcctgctttacagtagttctcaacaatctccgtgtgtttctcatatttgactggctactgttagtccatgattttctccacactcccagtgatattaagaaacaaaatcatgttactccttctcgccaccgtaactctagcagcgaatctgctatagttcccaaaactgtgaagagtggagtagttaccaagcggtcttcccttcctgtgtccaatgaaaggcacctggaggtcaaggtcaatgtaacaggtacggagtttgtggtcattgaagatgtgtcctgcttcgacaccaatgccattattctgaaaggcaccacagtgctcacctataagccccggtttgttgatcgccccttttcaggaagtttgtttggcattgaggtgttttcatgccgactagggaatgagcatgatacagctctttcaattgtggatcccgtacaaattcaaatggagttggtggggaattcttcttatcaaaatagttcaggattgatggatgcattcaatagtgaagatttcccacctgtcctggagattcagttacaagccctggatatcagactctcctataatgatgttcagctgtttcttgccattgcaaaatccatcccagagcaagctaatgctgcagtgccagactcagtggccctggagtcagactccgttggcacttaccttccaggtgcatctcgcgttggagaggaaatcagagaagggacaagacacaccttagatcctgtcttggagttacagctggctaggctgcaggagctgggattcagcatggatgattgtcgcaaagctcttttggcgtgtcaaggccaattgaaaaaggcagcaagttggttgtttaagaatgcggaacctctgaagtctctttccttggcctccaccagccgagatagcccaggggctgtggcagcgccattgatctctggcgtggagatcaaagctgagagtgtgtgcatctgtttcatcgatgactgcatggattgtgatgttcctctcgctgaactcaccttttcccgtctgaattttcttcagcgtgtaagaactagccctgaaggctatgcccacttcaccctttctggagattattataaccgtgctctttcaggctgggagccatttattgagccttggccatgctctgtatcctggcaacagcaggcagctagtcgtctccatcctcctcgactgaagctagaagccaaggccaaacctcgtttggatatcaatatcacttctgtgctaattgaccagtatgtaagtaccaaggaatcgtggatggcagattactgtaaagatgacaaggacatagagtcagctaaatcagaagactggatgggctcttcggtggatcctccatgttttggacaaacagaggtgaaaacccccaagcgccggcagccatttgtcccctttgctctgaggaaccacacggggtgcactttgtggtttgccaccctgaccaccacacccaccagagctgcactctctcacagtgggagtccaggggtagttccagaagggaacggaacatttctcgatgatactcacaatgttagtgaatggcgagaagtccttacaggtgaagagattccctttgaatttgaagcaagaggaaagttaagacacagacacacccatgacctccggattcatcaactgcaagtgagagtaaatggctgggagcaagtgagcccagtgtctgtggacaaagtcgggaccttttttcgatatgcagcaccagataaaaattcatcttcctctacgattggcagcccaagcagcagaacaaatattatacatccccaggtttatttctcttcactcccaccagtgcgggtggtctttgcagtgactatggaaggcagtgcacggaaagtcatcactgtccggtcagccctcattgtgaggaacagacttgagacaccaatggaactaagactggatagcccatcagctccagacaagccagtggtgcttcctgctatcatgccaggggattcgtttgctgtgcctttacacctcacttcttggcggctacaggcccggcccaaaggattgggtgtatttttctgtaaggctcccattcattggaccaatgtagtgaagactgcagaaattagtagcagtaaacgagagtgccactctatggacacagaaaaaagccgatttttcaggttttgtgtggctataaagaaagagaattatccagattatatgccctcaaacatattttctgacagtgcaaaacagattttcagacagcctgggcataccatatatctcctgccaactgtggtaatctgcaacttgctaccctgtgaacttgatttttatgttaaaggaatgccaattaatgggacgctgaaacctggcaaggaggcagctctccatacagctgatacatcccagaacattgagctgggggtatcactggagaatttccccctctgtaaagaattgctcattccacctggaacccaaaactatatggtgagaatgcgactctatgacgtcaaccgtcggcagctgaacctcaccatccggattgtgtgtcgagcagaaggatccttaaagatcttcatttctgctccatattggctgattaacaaaacagggttgccactgatcttcagacaggacaatgccaagacagatgctgcaggccagtttgaggagcatgagctggcccgtagcctgagtcctctcttattctgctatgctgacaaagagcagccaaacctctgcacgatgagaatcggaagggggattcatccagaaggcatgccgggctggtgtcagggcttctccctggatggtggtagtggtgtccgagctttgaaagtcatccagcaaggaaaccgcccagggctgatctataacattggtattgatgtcaagaaaggccgaggtcgatacattgatacctgcatggtcatctttgccccccgttacctgttagataataaatcatctcacaagcttgcatttgcacagagggaatttgccaggggacagggaacagccaatcccgaaggttacatttccacccttcctggttccagtgtggtgttccactggcctcggaatgactatgatcagctattgtgtgtcagactgatggacgttcccaattgtatttggtctggaggctttgaagtcaacaagaataattccttccatatcaacatgagggataccttgggaaaatgcttcttcctacgagtggaaattactctccgaggagctacgtataggatctcatttagtgacacagatcagttacctcctcctttccgaattgacaacttttctaaggtcccggttgtctttactcagcatggcgtagctgaacccaggctccggactgaagtgaagcccatgacttcattggattatgcctgggacgaacccaccttgccaccttttatcactctgactgttaaaggggcagggtcctctgagatcaactgcaacatgaatgatttccaggataatcggcagctttattatgaaaatttcatttacattgctgctacatatacattctctggcttgcaggagggaacaggcaggcctgtggcttccaacaaggccattacctgtgcggagctcgttttggatgtctcacccaagacacaaagagtcattttaaaaaagaaggaaccaggaaagcgttctcagctgtggaggatgacaggaacaggaatgctggcccatgagggctcctcagttcctcacaatcccaataagccctcagccgcccgctccaccgaggggtctgccatcttagatattgctggtctcgctgcagtgactgacaacagatacgagccactgatgctgagaaagcctgaccgcaggcgaagcacaactcagacgtggagtttccgagaaggaaaactgacctgtgggttacatgggttggtcgtccaggccaaaggaggactttctggtttgtttgatggagctgaagttgttcttggtcctgacacttccatggagcttttggggccagttccacctgaacaacaatttattaatcaaaaaatgagacctggttctggaatgttatccatcagagtcatcccagatggaccaactagagcactccagataacagatttctgccaccggaaaagcagccgttcatatgaagtggatgaacttcctgtcaccgaacaagagctgcagaaattaaagaatccagatacagagcaggaattggaagtgcttgtgaggttagaaggtggaattgggttgtccttaattaataaagtcccagaagaactggtctttgcaagtcttacaggaatcaatgtgcactatacacagctggcaaccagtcacatgcttgaactcagcatacaggatgtacaggtggacaatcagctcattggtaccacgcagcccttcatgctctatgtgactcccctgagcaatgagaatgaggtcatcgagaccggcccagctgtgcaagtcaacgcagtgaagttccccagtaagagtgcactgaccaacatctacaagcatctgatgatcacagctcagagattcacagtgcaaattgaggagaaactgctcctcaagctgctaagtttctttggctacgatcaagcagaatcagaggtggaaaaatatgatgaaaacctccatgaaaagacagctgagcaaggtggaacaccaattcgatactactttgaaaatctcaaaatcagcattcctcagatcaagctaagtgtgttcacctccaacaagctcccattggatcttaaggccctaaaaagcaccttggggtttcctttgatacggtttgaagacgctgtgattaatctagatccattcactcgggtacatccctatgagaccaaggagttcatcatcaatgatatcctcaaacatttccaggaggaactcctcagccaggcagctcgaatcctgggatcagtggattttcttggcaatcctatggggcttttgaatgatgtttctgaaggggttactggactgataaaatatggaaatgtcgggggcctcatcagaaatgttacacacggagtatcaaactctgctgccaagtttgctggaacattatcagatggcttagggaagacgatggacaatcggcatcagtcagagcgggagtacatcaggtaccatgcagccacaagtggtgaacaccttgtagccggcatccatggcctggctcatggtatcattggtggactgaccagtgttataacttcgacagtggaaggtgtgaaaacagaagggggtgtcagcggtttcatatctggccttggaaaagggcttgttggcactgtaaccaagccagtggcaggcgccctggattttgcatcagaaacagcccaggcggtgagagacacagccacactcagcggccccaggactcaagcacagagggttcggaaaccgcgttgctgcacggggccccaggggctgcttccccgatattctgagagccaggcggaaggacaggagcagctcttcaaactcacagacaacatacaggacgaattcttcatcgctgtggagaacattgacagctactgcgtgctcatctcctccaaagctgtttacttcctgaaaagtggagactacgtggatcgagaagccattttcctagaagtcaaatacgatgacctctaccactgccttgtctccaaagaccatgggaaggtgtatgtgcaggtgaccaagaaagccgtgagcacgagcagtggagtgtccatccccggcccctcccaccagaagcccatggtccatgtgaaatctgaggtccttgctgtcaagttgtcacaagaaataaactacgcaaagagcctctactatgaacagcagcttatgttaagactcagcgaaaaccgagagcagctggagctggactcctgaagccccgctgctgagatgggcgctcccgacacagcgcagacccaccaggaggaaagaggcccagctctcagctgacgatggaggcagaaccggagtcgggtttggggaagttgtcaaggaatgagggaaagtaaatcctcatgaggaaaagtacaaatggaaatcgtattaatttgtgaggcagggagttattttagattatgggaaataatttttaaaggtattggttaaataacgtttaaaaacatgtactgagatgaatctaatttttagattgccctgtattttgttaacatgtatatatgtacaacagtgtgtttgtaaatatataggaacgtttctgaacagggtctgtgctatgtgtaaaggtttgttaactgtaaagtaatataaagttatattggatcttctattgcactaattctagatgtctaattcaggatactgtctatagaaaggcattcttaaaagttaaagaatgttacgtcttagttttggagactaaagtattcccagtaaagtgggttgaggtgagggctgtggtcctgaaagggacgcctttgacatcgtggctgtccagttgggctgtgagctgtggcacccaggactggcgctggcccttcagaaggatctaggagaggggcttgggagcccacttttaatttctcacccccattttacaaagagtgcttagattcttacaaattatgatgtaagttatccatttggctttttcctaactagtcttaccaaacttagggggaaacctgtgctccattaccacatgggtgcaagtcagcattgtaagttttctcaggttattattattagagaggttggaaacattggtaaactctgttgattgagaaggaaaaaaaaagtcccattgaactgttgcaacaaatcagaaatccacataaaagtgctctcctgcctgggcagcaacaaccaagaacaaagccccgggactgttttctttttaataaagccacaggcaggcatcgtagctccacagcccgaggggacacaggatggaaaccccaggatgagaagggagcagggagagttccagaaagggggatgaaataggagtattaaaaagctgcgttggtaagtttttcatggaaccaagatttgacaaaggcatctcttatccttggttttaaattcctgctgggagcaaggcctggtatgagcgccctgggtcttgtttttggtgtttcgcttttctgtaaggattaagcagatagggagaagggaaaaggggcctcactttagaatgaatgagtcaccttgtgatttttaaatttttattttaataaagctaatcaatttctacaaccttgtcacatgtagctgagtctgggatgactcagtggatcagtggatgcgtggaaggttttggtgtttataactcatgacccaaatccttccaagacacaaatgaagctaatcttcttcccctccctttcctctcccacttcccctctgtgtctgtgtctgtgtgtgtgtgtgtgtgtgtgtgtgtgtgcgcgcgcgcgcgtgcatgcagagaggaaggaagggagctttagcacaagtagctacatagtgttcaaggatcagctttcttgaactccgaggcttggttgcctttgctgaactccaagttagtggattgcagaatggaaacttggcttttgcggcactgggtgagttttagtttgtgtgtgtcttgctggggggtggtgatgattgtctcagcactcacgcactgcacaagatggcagcaggatacagcactgcacaagatggcagctcctctgcagcttcctcctcagcctccctccttgcacccccacaggtttggcttgtggtttttgtcatcagtaacctactgcctgagatcatgatctcttaaaagatgagactctcggaagggttgattgtatgcgtcagtgagccttctatcaccttctggaacaaagtcacttgaaatctcttgatgagattaaggagtttagtgttactaagaaaatctgctttgggccgcagcagtgctgggtgttctcagacctgactgaggaagttagctgcgggctgccctgtgggctggtgcttcaggaggaatccagagaagtgttcagatgccccccttgggctcctttctaattttaatcagctctttaaatagctgcccatctcctgtgattgcacaaccaagcactttgacatttgcaccttaggagaggcagatgttaaaatggaatccaaagaccacctagggcggggctgggtgggagatgggagggccaactgcgagctgctccacttctcagctctcccctgccctgcagccctgggccagacaaggccagaaggtttcaggggcatttgacatcccctcctggttctcaccaggaaaacatccaaagctttggaggaaacaggccctgcccctggctccttaaatgccccgtctctttgtaaactgatattcagccagcaatgcctaagactttgttaagatcatttctactgcttttctttctgcttcaaacacacagttcgtctctgaggaaagtaaaataaatggaataagagtaaattgggtaaggagatatccaaagctacccagtcccttgacccagcacagttggccgacccgtgtcactccctggctgtcgctgcttctctgtgctcactgaagggtgagccaggccagtgcttccccagcccctgggcctggtcactacacagtggaaaacagacaagcggccccttccccaaatcccaagagtgtcttgctgcttggtgggtgctcatcgcaatgttctgaaggctccagggccactttgtttgtaagtatgatctgggcctcaaaataccatagtagctgcttgataaaattctaaaaatatctggttctctattatgtaaacactattacagtcaccagtgtgtgaagactcttgagtctggttctcatatcagagtcatcatttttcttcctgtggaataaaatgccttgtggacttcccaaa

SEQ ID NO: 3 - protein, 4388 aa, Homo Sapiens
MLEGLVAWVLNTYLGKYVNNLNTDQLSVALLKGAVELENLPLKKDALKELELPFEVKAGFIGKVTLQIPFYRPHVDPWVISISSLHLIGAPEKIQDFNDEKEKLLERERKKALLQALEEKWKNDRQQKGESYWYSVTASVVTRIVENIELKIQDVHLRFEDGVTNPSHPFAFGICIKNVSMQNAVNEPVQKLMRKKQLDVAEFSIYWDVDCTLLGDLPQMELQEAMARSMESRSHHYVLEPVFASALLKRNCSKKPLRSRHSPRIDCDIQLETIPLKLSQLQYRQIMEFLKELERKERQVKFRRWKPKVAISKNCREWWYFALNANLYEIREQRKRCTWDFMLHRARDAVSYTDKYFNKLKGGLLSTDDKEEMCRIEEEQSFEELKILRELVHDRFHKQEELAESLREPQFDSPGACPGAPEPGGGSGMLQYLQSWFPGWGGWYGQQTPEGNVVEGLSAEQQEQWIPEEILGTEEFFDPTADASCMNTYTKRDHVFAKLNLQLQRGTVTLLHKEQGTPQMNESAFMQLEFSDVKLLAESLPRRNSSLLSVRLGGLFLRDLATEGTMFPLLVFPNPQKEVGRVSQSFGLQTTSADRSDHYPAADPDGPVFEMLYERNPAHSHFERRLNVSTRPLNIIYNPQAIKKVADFFYKGKVHTSGFGYQSELELRVAEAARRQYNKLKMQTKAEIRQTLDRLLVGDFIEESKRWTVRLDISAPQVIFPDDFKFKNPVLVVVDLGRMLLTNTQDNSRRKSRDGSASEETQFSDDEYKTPLATPPNTPPPESSSSNGEKTPPFSGVEFSEEQLQAHLMSTKMYERYSLSFMDLQIMVGRVKDNWKHVQDIDVGPTHVVEKFNVHLQLERRLIYTSDPKYPGAVLSGNLPDLKIHINEDKISALKNCFALLTTPEMKTSDTQIKEKIFPQEEQRGSLQDSVMNLTQSIVLLEQHTREVLVESQLLLAEFKVNCMQLGVESNGRYISVLKVFGTNAHFVKRPYDAEVSLTVHGLLLVDTMQTYGADFDLLMASHKNLSFDIPTGSLRDSRAQSPVSGPNVAHLTDGATLNDRSATSVSLDKILTKEQESLIKLEYQFVSSECPSMNLDSTLQVISLQVNNLDIILNPETIVELIGFLQKSFPKEKDDLSPQPLMTDFERSFREQGTYQSTYEQNTEVAVEIHRLNLLLLRTVGMANREKYGRKIATASIGGTKVNVSMGSTFDMNGSLGCLQLMDLTQDNVKNQYVVSIGNSVGYENIISDIGYFESVFVRMEDAALTEALSFTFVERSKQECFLNLKMASLHYNHSAKFLKELTLSMDELEENFRGMLKSAATKVTTVLATKTAEYSEMVSLFETPRKTREPFILEENEIYGFDLASSHLDTVKLILNINIESPVVSIPRKPGSPELLVGHLGQIFIQNFVAGDDESRSDRLQVEIKDIKLYSLNCTQLAGREAVGSEGSRMFCPPSGSGSANSQEEAHFTRHDFFESLHRGQAFHILNNTTIQFKLEKIPIERESELTFSLSPDDLGTSSIMKIEGKFVNPVQVVLAKHVYEQVLQTLDNLVYSEDLNKYPASATSSPCPDSPLPPLSTCGESSVERKENGLFSHSSLSNTSQKSLSVKEVKSFTQIQATFCISELQVQLSGDLTLGAQGLVSLKFQDFEVEFSKDHPQTLSIQIALHSLLMEDLLEKNPDSKYKNLMVSRGAPKPSSLAQKEYLSQSCPSVSNVEYPDMPRSLPSHMEEAPNVFQLYQRPTSASRKKQKEVQDKDYPLTPPPSPTVDEPKILVGKSKFDDSLVHINIFLVDKKHPEFSSSYNRVNRSIDVDFNCLDVLITLQTWVVILDFFGIGSTADNHAMRLPPEGILHNVKLEPHASMESGLQDPVNTKLDLKVHSLSLVLNKTTSELAKANVSKLVAHLEMIEGDLALQGSIGSLSLSDLTCHGEFYRERFTTSGEEALIFQTFKYGRPDPLLRREHDIRVSLRMASVQYVHTQRFQAEVVAFIQHFTQLQDVLGRQRAAIEGQTVRDQAQRCSRVLLDIEAGAPVLLIPESSRSNNLIVANLGKLKVKNKFLFAGFPGTFSLQDKESVPSASPTGIPKHSLRKTTSTEEPRGTHSQGQFTMPLAGMSLGSLKSEFVPSTSTKQQGPQPTLSVGQESSSPEDHVCLLDCVVVDLQDMDIFAAERHPREYSKAPEDSSGDLIFPSYFVRQTGGSLLTEPCRLKLQVERNLDKEISHTVPDISIHGNLSSVHCSLDLYKYKLIRGLLENNLGEPIEEFMRPYDLQDPRIHTVLSGEVYTCMCFLIDMVNVSLELKDPKRKEGAGSLARFDFKKCKLLYESFSNQTKSINLVSHSMMAFDTRYAGQKTSPGMTNVFSCIFQPAKNSSTTQGSIQIELHFRSTKDSSCFTVVLNNLRVFLIFDWLLLVHDFLHTPSDIKKQNHVTPSRHRNSSSESAIVPKTVKSGVVTKRSSLPVSNERHLEVKVNVTGTEFVVIEDVSCFDTNAIILKGTTVLTYKPRFVDRPFSGSLFGIEVFSCRLGNEHDTALSIVDPVQIQMELVGNSSYQNSSGLMDAFNSEDFPPVLEIQLQALDIRLSYNDVQLFLAIAKSIPEQANAAVPDSVALESDSVGTYLPGASRVGEEIREGTRHTLDPVLELQLARLQELGFSMDDCRKALLACQGQLKKAASWLFKNAEPLKSLSLASTSRDSPGAVAAPLISGVEIKAESVCICFIDDCMDCDVPLAELTFSRLNFLQRVRTSPEGYAHFTLSGDYYNRALSGWEPFIEPWPCSVSWQQQAASRLHPPRLKLEAKAKPRLDINITSVLIDQYVSTKESWMADYCKDDKDIESAKSEDWMGSSVDPPCFGQSLPLVYLRTRSTASLTNLEHQIYARAEVKTPKRRQPFVPFALRNHTGCTLWFATLTTTPTRAALSHSGSPGVVPEGNGTFLDDTHNVSEWREVLTGEEIPFEFEARGKLRHRHTHDLRIHQLQVRVNGWEQVSPVSVDKVGTFFRYAAPDKNSSSSTIGSPSSRTNIIHPQVYFSSLPPVRVVFAVTMEGSARKVITVRSALIVRNRLETPMELRLDSPSAPDKPVVLPAIMPGDSFAVPLHLTSWRLQARPKGLGVFFCKAPIHWTNVVKTAEISSSKRECHSMDTEKSRFFRFCVAIKKENYPDYMPSNIFSDSAKQIFRQPGHTIYLLPTVVICNLLPCELDFYVKGMPINGTLKPGKEAALHTADTSQNIELGVSLENFPLCKELLIPPGTQNYMVRMRLYDVNRRQLNLTIRIVCRAEGSLKIFISAPYWLINKTGLPLIFRQDNAKTDAAGQFEEHELARSLSPLLFCYADKEQPNLCTMRIGRGIHPEGMPGWCQGFSLDGGSGVRALKVIQQGNRPGLIYNIGIDVKKGRGRYIDTCMVIFAPRYLLDNKSSHKLAFAQREFARGQGTANPEGYISTLPGSSVVFHWPRNDYDQLLCVRLMDVPNCIWSGGFEVNKNNSFHINMRDTLGKCFFLRVEITLRGATYRISFSDTDQLPPPFRIDNFSKVPVVFTQHGVAEPRLRTEVKPMTSLDYAWDEPTLPPFITLTVKGAGSSEINCNMNDFQDNRQLYYENFIYIAATYTFSGLQEGTGRPVASNKAITCAELVLDVSPKTQRVILKKKEPGKRSQLWRMTGTGMLAHEGSSVPHNPNKPSAARSTEGSAILDIAGLAAVTDNRYEPLMLRKPDRRRSTTQTWSFREGKLTCGLHGLVVQAKGGLSGLFDGAEVVLGPDTSMELLGPVPPEQQFINQKMRPGSGMLSIRVIPDGPTRALQITDFCHRKSSRSYEVDELPVTEQELQKLKNPDTEQELEVLVRLEGGIGLSLINKVPEELVFASLTGINVHYTQLATSHMLELSIQDVQVDNQLIGTTQPFMLYVTPLSNENEVIETGPAVQVNAVKFPSKSALTNIYKHLMITAQRFTVQIEEKLLLKLLSFFGYDQAESEVEKYDENLHEKTAEQGGTPIRYYFENLKISIPQIKLSVFTSNKLPLDLKALKSTLGFPLIRFEDAVINLDPFTRVHPYETKEFIINDILKHFQEELLSQAARILGSVDFLGNPMGLLNDVSEGVTGLIKYGNVGGLIRNVTHGVSNSAAKFAGTLSDGLGKTMDNRHQSEREYIRYHAATSGEHLVAGIHGLAHGIIGGLTSVITSTVEGVKTEGGVSGFISGLGKGLVGTVTKPVAGALDFASETAQAVRDTATLSGPRTQAQRVRKPRCCTGPQGLLPRYSESQAEGQEQLFKLTDNIQDEFFIAVENIDSYCVLISSKAVYFLKSGDYVDREAIFLEVKYDDLYHCLVSKDHGKVYVQVTKKAVSTSSGVSIPGPSHQKPMVHVKSEVLAVKLSQEINYAKSLYYEQQLMLRLSENREQLELDS

SEQ ID NO: 4 - protein, 4363 aa, Homo Sapiens
MLEGLVAWVLNTYLGKYVNNLNTDQLSVALLKGAVELENLPLKKDALKELELPFEVKAGFIGKVTLQIPFYRPHVDPWVISISSLHLIGAPEKIQDFNDEKEKLLERERKKALLQALEEKWKNDRQQKGESYWYSVTASVVTRIVENIELKIQDVHLRFEDGVTNPSHPFAFGICIKNVSMQNAVNEPVQKLMRKKQLDVAEFSIYWDVDCTLLGDLPQMELQEAMARSMESRSHHYVLEPVFASALLKRNCSKKPLRSRHSPRIDCDIQLETIPLKLSQLQYRQIMEFLKELERKERQVKFRRWKPKVAISKNCREWWYFALNANLYEIREQRKRCTWDFMLHRARDAVSYTDKYFNKLKGGLLSTDDKEEMCRIEEEQSFEELKILRELVHDRFHKQEELAESLREPQFDSPGACPGAPEPGGGSGMLQYLQSWFPGWGGWYGQQTPEGNVVEGLSAEQQEQWIPEEILGTEEFFDPTADASCMNTYTKRDHVFAKLNLQLQRGTVTLLHKEQGTPQMNESAFMQLEFSDVKLLAESLPRRNSSLLSVRLGGLFLRDLATEGTMFPLLVFPNPQKEVGRVSQSFGLQTTSADRSDHYPAADPDGPVFEMLYERNPAHSHFERRLNVSTRPLNIIYNPQAIKKVADFFYKGKVHTSGFGYQSELELRVAEAARRQYNKLKMQTKAEIRQTLDRLLVGDFIEESKRWTVRLDISAPQVIFPDDFKFKNPVLVVVDLGRMLLTNTQDNSRRKSRDGSASEETQFSDDEYKTPLATPPNTPPPESSSSNGEKTPPFSGVEFSEEQLQAHLMSTKMYERYSLSFMDLQIMVGRVKDNWKHVQDIDVGPTHVVEKFNVHLQLERRLIYTSDPKYPGAVLSGNLPDLKIHINEDKISALKNCFALLTTPEMKTSDTQIKEKIFPQEEQRGSLQDSVMNLTQSIVLLEQHTREVLVESQLLLAEFKVNCMQLGVESNGRYISVLKVFGTNAHFVKRPYDAEVSLTVHGLLLVDTMQTYGADFDLLMASHKNLSFDIPTGSLRDSRAQSPVSGPNVAHLTDGATLNDRSATSVSLDKILTKEQESLIKLEYQFVSSECPSMNLDSTLQVISLQVNNLDIILNPETIVELIGFLQKSFPKEKDDLSPQPLMTDFERSFREQGTYQSTYEQNTEVAVEIHRLNLLLLRTVGMANREKYGRKIATASIGGTKVNVSMGSTFDMNGSLGCLQLMDLTQDNVKNQYVVSIGNSVGYENIISDIGYFESVFVRMEDAALTEALSFTFVERSKQECFLNLKMASLHYNHSAKFLKELTLSMDELEENFRGMLKSAATKVTTVLATKTAEYSEMVSLFETPRKTREPFILEENEIYGFDLASSHLDTVKLILNINIESPVVSIPRKPGSPELLVGHLGQIFIQNFVAGDDESRSDRLQVEIKDIKLYSLNCTQLAGREAVGSEGSRMFCPPSGSGSANSQEEAHFTRHDFFESLHRGQAFHILNNTTIQFKLEKIPIERESELTFSLSPDDLGTSSIMKIEGKFVNPVQVVLAKHVYEQVLQTLDNLVYSEDLNKYPASATSSPCPDSPLPPLSTCGESSVERKENGLFSHSSLSNTSQKSLSVKEVKSFTQIQATFCISELQVQLSGDLTLGAQGLVSLKFQDFEVEFSKDHPQTLSIQIALHSLLMEDLLEKNPDSKYKNLMVSRGAPKPSSLAQKEYLSQSCPSVSNVEYPDMPRSLPSHMEEAPNVFQLYQRPTSASRKKQKEVQDKDYPLTPPPSPTVDEPKILVGKSKFDDSLVHINIFLVDKKHPEFSSSYNRVNRSIDVDFNCLDVLITLQTWVVILDFFGIGSTADNHAMRLPPEGILHNVKLEPHASMESGLQDPVNTKLDLKVHSLSLVLNKTTSELAKANVSKLVAHLEMIEGDLALQGSIGSLSLSDLTCHGEFYRERFTTSGEEALIFQTFKYGRPDPLLRREHDIRVSLRMASVQYVHTQRFQAEVVAFIQHFTQLQDVLGRQRAAIEGQTVRDQAQRCSRVLLDIEAGAPVLLIPESSRSNNLIVANLGKLKVKNKFLFAGFPGTFSLQDKESVPSASPTGIPKHSLRKTTSTEEPRGTHSQGQFTMPLAGMSLGSLKSEFVPSTSTKQQGPQPTLSVGQESSSPEDHVCLLDCVVVDLQDMDIFAAERHPREYSKAPEDSSGDLIFPSYFVRQTGGSLLTEPCRLKLQVERNLDKEISHTVPDISIHGNLSSVHCSLDLYKYKLIRGLLENNLGEPIEEFMRPYDLQDPRIHTVLSGEVYTCMCFLIDMVNVSLELKDPKRKEGAGSLARFDFKKCKLLYESFSNQTKSINLVSHSMMAFDTRYAGQKTSPGMTNVFSCIFQPAKNSSTTQGSIQIELHFRSTKDSSCFTVVLNNLRVFLIFDWLLLVHDFLHTPSDIKKQNHVTPSRHRNSSSESAIVPKTVKSGVVTKRSSLPVSNERHLEVKVNVTGTEFVVIEDVSCFDTNAIILKGTTVLTYKPRFVDRPFSGSLFGIEVFSCRLGNEHDTALSIVDPVQIQMELVGNSSYQNSSGLMDAFNSEDFPPVLEIQLQALDIRLSYNDVQLFLAIAKSIPEQANAAVPDSVALESDSVGTYLPGASRVGEEIREGTRHTLDPVLELQLARLQELGFSMDDCRKALLACQGQLKKAASWLFKNAEPLKSLSLASTSRDSPGAVAAPLISGVEIKAESVCICFIDDCMDCDVPLAELTFSRLNFLQRVRTSPEGYAHFTLSGDYYNRALSGWEPFIEPWPCSVSWQQQAASRLHPPRLKLEAKAKPRLDINITSVLIDQYVSTKESWMADYCKDDKDIESAKSEDWMGSSVDPPCFGQTEVKTPKRRQPFVPFALRNHTGCTLWFATLTTTPTRAALSHSGSPGVVPEGNGTFLDDTHNVSEWREVLTGEEIPFEFEARGKLRHRHTHDLRIHQLQVRVNGWEQVSPVSVDKVGTFFRYAAPDKNSSSSTIGSPSSRTNIIHPQVYFSSLPPVRVVFAVTMEGSARKVITVRSALIVRNRLETPMELRLDSPSAPDKPVVLPAIMPGDSFAVPLHLTSWRLQARPKGLGVFFCKAPIHWTNVVKTAEISSSKRECHSMDTEKSRFFRFCVAIKKENYPDYMPSNIFSDSAKQIFRQPGHTIYLLPTVVICNLLPCELDFYVKGMPINGTLKPGKEAALHTADTSQNIELGVSLENFPLCKELLIPPGTQNYMVRMRLYDVNRRQLNLTIRIVCRAEGSLKIFISAPYWLINKTGLPLIFRQDNAKTDAAGQFEEHELARSLSPLLFCYADKEQPNLCTMRIGRGIHPEGMPGWCQGFSLDGGSGVRALKVIQQGNRPGLIYNIGIDVKKGRGRYIDTCMVIFAPRYLLDNKSSHKLAFAQREFARGQGTANPEGYISTLPGSSVVFHWPRNDYDQLLCVRLMDVPNCIWSGGFEVNKNNSFHINMRDTLGKCFFLRVEITLRGATYRISFSDTDQLPPPFRIDNFSKVPVVFTQHGVAEPRLRTEVKPMTSLDYAWDEPTLPPFITLTVKGAGSSEINCNMNDFQDNRQLYYENFIYIAATYTFSGLQEGTGRPVASNKAITCAELVLDVSPKTQRVILKKKEPGKRSQLWRMTGTGMLAHEGSSVPHNPNKPSAARSTEGSAILDIAGLAAVTDNRYEPLMLRKPDRRRSTTQTWSFREGKLTCGLHGLVVQAKGGLSGLFDGAEVVLGPDTSMELLGPVPPEQQFINQKMRPGSGMLSIRVIPDGPTRALQITDFCHRKSSRSYEVDELPVTEQELQKLKNPDTEQELEVLVRLEGGIGLSLINKVPEELVFASLTGINVHYTQLATSHMLELSIQDVQVDNQLIGTTQPFMLYVTPLSNENEVIETGPAVQVNAVKFPSKSALTNIYKHLMITAQRFTVQIEEKLLLKLLSFFGYDQAESEVEKYDENLHEKTAEQGGTPIRYYFENLKISIPQIKLSVFTSNKLPLDLKALKSTLGFPLIRFEDAVINLDPFTRVHPYETKEFIINDILKHFQEELLSQAARILGSVDFLGNPMGLLNDVSEGVTGLIKYGNVGGLIRNVTHGVSNSAAKFAGTLSDGLGKTMDNRHQSEREYIRYHAATSGEHLVAGIHGLAHGIIGGLTSVITSTVEGVKTEGGVSGFISGLGKGLVGTVTKPVAGALDFASETAQAVRDTATLSGPRTQAQRVRKPRCCTGPQGLLPRYSESQAEGQEQLFKLTDNIQDEFFIAVENIDSYCVLISSKAVYFLKSGDYVDREAIFLEVKYDDLYHCLVSKDHGKVYVQVTKKAVSTSSGVSIPGPSHQKPMVHVKSEVLAVKLSQEINYAKSLYYEQQLMLRLSENREQLELDS

SEQ ID NO: 5 - DNA, 5212 bp, Homo Sapiens
actccccctgcctcctctccgcctttaacttctcgggaagatgaggcagtttggcatctgtggccgagttgctgttgccgggtgatagttggagcggagacttagcataatggcagaacctgtttctccactgaagcactttgtgctggctaagaaggcgattactgcaatctttgaccagttactggagtttgttactgaaggatcacattttgttgaagcaacatataagaatccggaacttgatcgaatagccactgaagatgatctggtagaaatgcaaggatataaagacaagctttccatcattggtgaggtgctatctcggagacacatgaaggtggcattttttggcaggacaagcagtgggaagagctctgttatcaatgcaatgttgtgggataaagttctccctagtgggattggccatataaccaattgcttcctaagtgttgaaggaactgatggagataaagcctatcttatgacagaaggatcagatgaaaaaaagagtgtgaagacagttaatcaactggcccatgcccttcacatggacaaagatttgaaagctggctgtcttgtacgtgtgttttggccaaaagcaaaatgtgccctcttgagagatgacctggtgttagtagacagtccaggcacagatgtcactacagagctggatagctggattgataagttttgcctagatgctgatgtctttgttttggtcgcaaactctgaatcaacactaatgaatacggaaaaacacttttttcacaaggtgaatgagcggctttccaagcctaatattttcattctcaataatcgttgggatgcctctgcatcagagccagaatatatggaagacgtacgcagacagcacatggaaagatgcctgcatttcttggtggaggagctcaaagttgtaaatgctttagaagcacagaatcgtatcttctttgtttcagcaaaggaagttcttagtgctagaaagcaaaaagcacaggggatgccagaaagtggtgtggcacttgctgaaggatttcatgcaagattacaggaatttcagaattttgaacaaatctttgaggagtgtatctcgcagtcagcagtgaaaacaaagttcgaacagcacactatcagagctaaacagatactagctactgtgaaaaacataatggattcagtaaacctggcagctgaagataaaaggcattattcagtggaagagagggaagaccaaattgatagactggactttattcgaaaccagatgaaccttttaacactggatgttaagaaaaaaatcaaggaggttaccgaggaggtggcaaacaaagtttcatgtgcaatgacagatgaaatttgtcgactgtctgttttggttgatgaattttgttcagagtttcatcctaatccagatgtattaaaaatatataaaagtgaattaaataagcacatagaggatggtatgggaagaaatttggctgatcgatgcaccgatgaagtaaacgccttagtgcttcagacccagcaagaaattattgaaaatttgaagccattacttccagctggtatacaggataaactacatacactgatcccttgcaagaaatttgatctcagttataatctaaattaccacaagttatgttcagattttcaagaggatattgtatttcgtttttccctgggctggtcttcccttgtacatcgatttttgggccctagaaatgctcaaagggtgctcctaggattatcagagcctatctttcagctccctagatctttagcttctactcccactgctcctaccactccagcaacgccagataatgcatcacaggaagaactcatgattacattagtaacaggattggcgtccgttacatctagaacttctatgggcatcattattgttggaggagtgatttggaaaactataggctggaaactcctatctgtttcattaactatgtatggagctttgtatctttatgaaagactgagctggaccacccatgccaaggagcgagcctttaaacagcagtttgtaaactatgcaactgaaaaactgaggatgattgttagctccacgagtgcaaactgcagtcaccaagtaaaacaacaaatagctaccacttttgctcgcctgtgccaacaagttgatattactcaaaaacagctggaagaagaaattgctagattacccaaagaaatagatcagttggagaaaatacaaaacaattcaaagctcttaagaaataaagctgttcaacttgaaaatgagctggagaattttactaagcagtttctaccttcaagcaatgaagaatcctaacaatagagattgctttggtgaccatgataggaggaaacgaaacttgtaagattggaacagttgttatttttatgaaattactttaaatatgaattgtactaactgtacctaaatagcaaagccctgtgtagattctggtaatgatctgtctcagggtatgtgtatttttgaagagtgttatgtccttagttttaattttgagtaaagaaaaggctaaaatcatgaattagttacaagcaacagtaccaacttatgtgacccctgaggggtggggctgtgagctcttaatttgtttttgattctgaaaaactctgcttcctggcatccaggagttagagattgagcctttcatcttctttctcaaaactagtttttgatgctttctttcatgggaatagtcacttttttatttagtaaatcgcattgctggaaccaccaaggagtgtggaatgtccttgagtgtattatttatgcaagtcacagtcacgttgccatcatggcagctatgtgaaacactaataaatgtgtttttactttttattcccgttaaaactgatgtaaaacaggataaaggcttgttatagtcacttataagtatctgggtctaagtaatttccttagatgtttctaaagaaacattttcagctttgctcccattatgattccaataaggaacgctttcctagtgcaattttaggagtaaagtttgaagagataaaaatagccaaagataggagacgtctgaattttgaatgataaacagtgatgttttaaaaaagctgttgttcttcaggaggcatttgcctaggatattgctggattataccccattggaggcttttaattttatttgtatgaattttccaggatttcattaaaaattattattgtattttttaccttaatgaaagattttgggttcaaatatctttctatattaaaagctgattgagtctgtacatatgtaaattatgcctagtggaggttctgttgactttcttccccactgtggaagaggccagttttgcctccatttgcacattcatttcagttatttctgatccataaatataacatttacaaaattcttccttgagctggtggaaatgcctcaccagtttcctctttaatgaatcaaataaaatctttaactgatgttaaaaaaaattgattgaaactcagatggaatggaaatgtacaaaaatgacaccattctaggaatttgctagacaaaatgtaggactaccagatcagtatctcctagacacttgttagaaatgcacaatcccggggaacgcagtacatttggccacatgtagtttatgtttccttttcatgggagggataaaatttaaagctttttttttctttgaatacagtccttccttttctatgccttttaaggcttctagatgctattcagcctttttacagcaggtgcaactctatttttcaaggtatcttagaagataacactaggccattgaagcctttcaaaaatatatttttatgcaaattgacacgagtgcagtatactaatgcaaattaattttggtgtttagcttttattgctcatttataacccaagaaatggtacagaccacagtaagtgggattaggtaacaactacaacatggaaaaacagtgtctaagatcacgtactgctaatttaggttacaaaactcaaggctgtaacctttatatgaaagtacttaagcttgaaagttcataatagtttggtatcacctcattagtatagtacagtggttctcaaagtttggtcctgggtcatcgacattacttctttttctttctgagacagggtctcagtctgtcacccaggatggagtgcagtggcgtgatcatggctcactgcagcctcaacctctcgggttcaggtgatcctcccacatcagcctcctgggtggctgggactacaggcacacgccaccatgcctggctagtttttgtaaagacgaggtttccccatgttgtgcaggctggtcttgaactcacagactcaagtgatccttttacctcagcctcccaaactgttgggataacaggtgtaagccaccacacacagctataatcaaccttcaaacttataaaaagtgtggatccttgggtctgaacccagacctatggagtcagacagtaggtttgaggcccagcaatctatggtttaacaagccatccaggtgtttctgatgcacagtgaaattggggtaccactggtattaggtttggtatggcaactttttcatcacttgttttatgtagttgtctgatcaattgtgaaaacataatgaatgttggaaatggaacagtaaaataacgaaagccaactttttttttttttttttttttttttgagacggagtctcgctctgtcgcccaggctggagtgcagtggcgcgatctcggctcactgcaagctccgcctcccgggttcacgccattctcctgcctcagcctcccgagtagctgggactacaggcgcccgccaccacgcccggctaattttttgtatttttagtagagacggggtttcaccgtgttagccaggatggtctcgatctcctgacctcgtgatccacccgcctcggcctcccaaagtgctgggattacaggcgtgagccaccgcgcccggccgaaagccaactcttatgcctagaaatatgtgcacctatgaccaagcccatgaattatacaggaattatgtaattatgagtgatgtacttcaaagttattgcacatacacttgtttactttgtatgtttgcaggattaaactttgtataatctttttacaaaaattttttttcagtatgcaagcttgcaagatgaaaataaaacctgtttgcctgatagttga

SEQ ID NO: 6 - protein, 741 aa, Homo Sapiens
MAEPVSPLKHFVLAKKAITAIFDQLLEFVTEGSHFVEATYKNPELDRIATEDDLVEMQGYKDKLSIIGEVLSRRHMKVAFFGRTSSGKSSVINAMLWDKVLPSGIGHITNCFLSVEGTDGDKAYLMTEGSDEKKSVKTVNQLAHALHMDKDLKAGCLVRVFWPKAKCALLRDDLVLVDSPGTDVTTELDSWIDKFCLDADVFVLVANSESTLMNTEKHFFHKVNERLSKPNIFILNNRWDASASEPEYMEDVRRQHMERCLHFLVEELKVVNALEAQNRIFFVSAKEVLSARKQKAQGMPESGVALAEGFHARLQEFQNFEQIFEECISQSAVKTKFEQHTIRAKQILATVKNIMDSVNLAAEDKRHYSVEEREDQIDRLDFIRNQMNLLTLDVKKKIKEVTEEVANKVSCAMTDEICRLSVLVDEFCSEFHPNPDVLKIYKSELNKHIEDGMGRNLADRCTDEVNALVLQTQQEIIENLKPLLPAGIQDKLHTLIPCKKFDLSYNLNYHKLCSDFQEDIVFRFSLGWSSLVHRFLGPRNAQRVLLGLSEPIFQLPRSLASTPTAPTTPATPDNASQEELMITLVTGLASVTSRTSMGIIIVGGVIWKTIGWKLLSVSLTMYGALYLYERLSWTTHAKERAFKQQFVNYATEKLRMIVSSTSANCSHQVKQQIATTFARLCQQVDITQKQLEEEIARLPKEIDQLEKIQNNSKLLRNKAVQLENELENFTKQFLPSSNEES

SEQ ID NO: 7 - DNA, 4262 bp, Homo Sapiens
gtagtgagtgtgatggccgccgcgaggccgggaaggtgaagcgcaatgtccctgctcttctctcgatgcaactctatcgtcacagtcaagaaaaataagagacacatggctgaggtgaatgcatccccacttaagcactttgtcactgccaagaagaagatcaatggcatttttgagcagctgggggcctacatccaggagagcgccaccttccttgaagacacgtacaggaatgcagaactggaccccgttaccacagaagaacaggttctggacgtcaaaggttacctatccaaagtgagaggcatcagtgaggtgctggctcggaggcacatgaaagtggctttttttggccggacgagcaatgggaagagcaccgtgatcaatgccatgctctgggacaaagttctgccctctgggattggccacaccaccaattgcttcctgcgggtagagggcacagatggccatgaggcctttctccttaccgagggctcagaggaaaagaggagtgccaagactgtgaaccagctggcccatgccctccaccaggacaagcagctccatgccggcagcctagtgagtgtgatgtggcccaactctaagtgcccacttctgaaggatgacctcgttttgatggacagccctggtattgatgtcaccacagagctggacagctggattgacaagttttgtctggatgctgatgtgtttgtgctggtggccaactcagagtccaccctgatgcagacggaaaagcacttcttccacaaggtgagtgagcgtctctcccggccaaacatcttcatcctgaacaaccgctgggatgcatctgcctcagagcccgagtacatggaggaggtgcggcggcagcacatggagcgttgtaccagcttcctggtggatgagctgggcgtggtggatcgatcccaggccggggaccgcatcttctttgtgtctgctaaggaggtgctcaacgccaggattcagaaagcccagggcatgcctgaaggagggggcgctctcgcagaaggctttcaagtgaggatgtttgagtttcagaattttgagaggagatttgaggagtgcatctcccagtctgcagtgaagaccaagtttgagcagcacacggtccgggccaagcagattgcagaggcggttcgactcatcatggactccctgcacatggcggctcgggagcagcaggtttactgcgaggaaatgcgtgaagagcggcaagaccgactgaaatttattgacaaacagctggagctcttggctcaagactataagctgcgaattaagcagattacggaggaagtggagaggcaggtgtcgactgcaatggccgaggagatcaggcgcctctctgtactggtggacgattaccagatggacttccacccttctccagtagtcctcaaggtttataagaatgagctgcaccgccacatagaggaaggactgggtcgaaacatgtctgaccgctgctccacggccatcaccaactccctgcagaccatgcagcaggacatgatagatggcttgaaacccctccttcctgtgtctgtgcggagtcagatagacatgctggtcccacgccagtgcttctccctcaactatgacctaaactgtgacaagctgtgtgctgacttccaggaagacattgagttccatttctctctcggatggaccatgctggtgaataggttcctgggccccaagaacagccgtcgggccttgatgggctacaatgaccaggtccagcgtcccatccctctgacgccagccaaccccagcatgcccccactgccacagggctcgctcacccaggaggagttcatggtttccatggttaccggcctggcctccttgacatccaggacctccatgggcattcttgttgttggaggagtggtgtggaaggcagtgggctggcggctcattgccctctcctttgggctctatggcctcctctacgtctatgagcgtctgacctggaccaccaaggccaaggagagggccttcaagcgccagtttgtggagcatgccagcgagaagctgcagcttgtcatcagctacactggctccaactgcagccaccaagtccagcaggaactgtctgggacctttgctcatctgtgtcagcaagttgacgtcacccgggagaacctggagcaggaaattgccgccatgaacaagaaaattgaggttcttgactcacttcagagcaaagcaaagctgctcaggaataaagccggttggttggacagtgagctcaacatgttcacacaccagtacctgcagcccagcagatagtgggcacctgaggcggagtctgcgtggagaggggcggtgctgccagccctaagtgccatgtgggctcccccaggggcacgtgtggctcctgccccctggccactgccaagagaatgaagcacccagtctcgtaccattttgagccctccagcactacttattttcccccacctttgcctgctgttgctggaagagctggctcatacccccaaaggacactttcagcgacagctatggacagcatggtaccaaggagttaagttgaggctttttccagctttctctggttcatttgattgcttgataaggcctcaggatctcagcattgcacaatgcctcatggaagcctttgagggtatcacacagacacccccaccttcctccagcctgtgcgcacctgccctccttgcagcccagcacacctgcaggtgtaagggacgattggagtttcttcccagagagtctgtcccagaaggactgtggcttgtgtgtgtccatctcgcctgttggctcagtgcttcatcccatttgcagagcctcagacacgtcttggtggtgaggctcagttacccctgggcttaggctgaggcgggccctgtgctgggggtggtagaaaggatgctgctgaggcagctggaggagtgggagtagctcagaggggagggctgttggatgtatggggagctggcagagcaggtggcagtcactgggacaaggagggacttgcctctcttctcattattgtgtcctttgctttagtgtcagtcctggacttgtgcaggcctgttttgtgtagatctgttttggaagatggcatggtctaggtggttgaaggatgtagtagaaggatggatggtggaaggtggggacgttggtggctggctgaggtgcatgggccccacacaggacagctggagaatgggccgtccacttggcctcgttctgcgaggggctcatgggtctgagagcccccacccactaggcttgattgcatccctgttgtgccctttaagagacatgtttccaccccacccccaaccttgtcccaagtgccctggactaaatttcctgtgccagtgactgcagttggccaagggacaatgtggaaaacccagtgtccatctttccaccctccctgatctccagaaccttcgactgacccccttgtctttatgctgatgttgagttttgggattgttactggttgaagtgggggcagatgcctgtcaccaaggtgttgactgtgtgagaaaagcagtttgggtgacaaatcctgtgtggcacaagttggatcgcttcctagaaataagcaacacctctcccaaaaagcagcccacaaggcaggggcccagcagcccagccatcactcatctttgaggaaatgagttggtagcctctgtgcactgtttggtggccacatcacaggtgatgtcctgttcacatacctgcttgtatttaaagccctcagtctgtcctgttgtgtggggcgaagtgatggactctgccaggtggacatgctgtgggtggatgttcccggcgtgtgccgggcctgaatggacaggggccacttcacagcatgtcagggaaaatcactgtcacacaattccaatggattttgtgctctttttgaaaaaaaaaaattctttagcgtaaacatgaattttttttcaatgtagcccctggggaatgaatgaaattttgagcttcttcaatacgtaaaattaaatttataccactgagggagagaccctttctgaaagaagtatggccaaaagcactttaatgctgctgacattgttgtttttatgttcatttgctggagcgcaagacgtgctgacacagtgagttttctctgatgtatttaaggtgatgtatttgcttgagttactcctgtatcattgctcataatattggaaactaaaataaaacctagttggaaatcc

SEQ ID NO: 8 - DNA, 4407 bp, Homo Sapiens
gtagtgagtgtgatggccgccgcgaggccgggaaggtgaagtcaggactggtggagtcaacacagtcaatcaatagccaacctcaacctgagacaggacagaagagaactcagaatctttttgtcttttggacttcagccatgtccatgatgcctaccctgtgaagatctctcaccatccaaaaaacgcaatgtccctgctcttctctcgatgcaactctatcgtcacagtcaagaaaaataagagacacatggctgaggtgaatgcatccccacttaagcactttgtcactgccaagaagaagatcaatggcatttttgagcagctgggggcctacatccaggagagcgccaccttccttgaagacacgtacaggaatgcagaactggaccccgttaccacagaagaacaggttctggacgtcaaaggttacctatccaaagtgagaggcatcagtgaggtgctggctcggaggcacatgaaagtggctttttttggccggacgagcaatgggaagagcaccgtgatcaatgccatgctctgggacaaagttctgccctctgggattggccacaccaccaattgcttcctgcgggtagagggcacagatggccatgaggcctttctccttaccgagggctcagaggaaaagaggagtgccaagactgtgaaccagctggcccatgccctccaccaggacaagcagctccatgccggcagcctagtgagtgtgatgtggcccaactctaagtgcccacttctgaaggatgacctcgttttgatggacagccctggtattgatgtcaccacagagctggacagctggattgacaagttttgtctggatgctgatgtgtttgtgctggtggccaactcagagtccaccctgatgcagacggaaaagcacttcttccacaaggtgagtgagcgtctctcccggccaaacatcttcatcctgaacaaccgctgggatgcatctgcctcagagcccgagtacatggaggaggtgcggcggcagcacatggagcgttgtaccagcttcctggtggatgagctgggcgtggtggatcgatcccaggccggggaccgcatcttctttgtgtctgctaaggaggtgctcaacgccaggattcagaaagcccagggcatgcctgaaggagggggcgctctcgcagaaggctttcaagtgaggatgtttgagtttcagaattttgagaggagatttgaggagtgcatctcccagtctgcagtgaagaccaagtttgagcagcacacggtccgggccaagcagattgcagaggcggttcgactcatcatggactccctgcacatggcggctcgggagcagcaggtttactgcgaggaaatgcgtgaagagcggcaagaccgactgaaatttattgacaaacagctggagctcttggctcaagactataagctgcgaattaagcagattacggaggaagtggagaggcaggtgtcgactgcaatggccgaggagatcaggcgcctctctgtactggtggacgattaccagatggacttccacccttctccagtagtcctcaaggtttataagaatgagctgcaccgccacatagaggaaggactgggtcgaaacatgtctgaccgctgctccacggccatcaccaactccctgcagaccatgcagcaggacatgatagatggcttgaaacccctccttcctgtgtctgtgcggagtcagatagacatgctggtcccacgccagtgcttctccctcaactatgacctaaactgtgacaagctgtgtgctgacttccaggaagacattgagttccatttctctctcggatggaccatgctggtgaataggttcctgggccccaagaacagccgtcgggccttgatgggctacaatgaccaggtccagcgtcccatccctctgacgccagccaaccccagcatgcccccactgccacagggctcgctcacccaggaggagttcatggtttccatggttaccggcctggcctccttgacatccaggacctccatgggcattcttgttgttggaggagtggtgtggaaggcagtgggctggcggctcattgccctctcctttgggctctatggcctcctctacgtctatgagcgtctgacctggaccaccaaggccaaggagagggccttcaagcgccagtttgtggagcatgccagcgagaagctgcagcttgtcatcagctacactggctccaactgcagccaccaagtccagcaggaactgtctgggacctttgctcatctgtgtcagcaagttgacgtcacccgggagaacctggagcaggaaattgccgccatgaacaagaaaattgaggttcttgactcacttcagagcaaagcaaagctgctcaggaataaagccggttggttggacagtgagctcaacatgttcacacaccagtacctgcagcccagcagatagtgggcacctgaggcggagtctgcgtggagaggggcggtgctgccagccctaagtgccatgtgggctcccccaggggcacgtgtggctcctgccccctggccactgccaagagaatgaagcacccagtctcgtaccattttgagccctccagcactacttattttcccccacctttgcctgctgttgctggaagagctggctcatacccccaaaggacactttcagcgacagctatggacagcatggtaccaaggagttaagttgaggctttttccagctttctctggttcatttgattgcttgataaggcctcaggatctcagcattgcacaatgcctcatggaagcctttgagggtatcacacagacacccccaccttcctccagcctgtgcgcacctgccctccttgcagcccagcacacctgcaggtgtaagggacgattggagtttcttcccagagagtctgtcccagaaggactgtggcttgtgtgtgtccatctcgcctgttggctcagtgcttcatcccatttgcagagcctcagacacgtcttggtggtgaggctcagttacccctgggcttaggctgaggcgggccctgtgctgggggtggtagaaaggatgctgctgaggcagctggaggagtgggagtagctcagaggggagggctgttggatgtatggggagctggcagagcaggtggcagtcactgggacaaggagggacttgcctctcttctcattattgtgtcctttgctttagtgtcagtcctggacttgtgcaggcctgttttgtgtagatctgttttggaagatggcatggtctaggtggttgaaggatgtagtagaaggatggatggtggaaggtggggacgttggtggctggctgaggtgcatgggccccacacaggacagctggagaatgggccgtccacttggcctcgttctgcgaggggctcatgggtctgagagcccccacccactaggcttgattgcatccctgttgtgccctttaagagacatgtttccaccccacccccaaccttgtcccaagtgccctggactaaatttcctgtgccagtgactgcagttggccaagggacaatgtggaaaacccagtgtccatctttccaccctccctgatctccagaaccttcgactgacccccttgtctttatgctgatgttgagttttgggattgttactggttgaagtgggggcagatgcctgtcaccaaggtgttgactgtgtgagaaaagcagtttgggtgacaaatcctgtgtggcacaagttggatcgcttcctagaaataagcaacacctctcccaaaaagcagcccacaaggcaggggcccagcagcccagccatcactcatctttgaggaaatgagttggtagcctctgtgcactgtttggtggccacatcacaggtgatgtcctgttcacatacctgcttgtatttaaagccctcagtctgtcctgttgtgtggggcgaagtgatggactctgccaggtggacatgctgtgggtggatgttcccggcgtgtgccgggcctgaatggacaggggccacttcacagcatgtcagggaaaatcactgtcacacaattccaatggattttgtgctctttttgaaaaaaaaaaattctttagcgtaaacatgaattttttttcaatgtagcccctggggaatgaatgaaattttgagcttcttcaatacgtaaaattaaatttataccactgagggagagaccctttctgaaagaagtatggccaaaagcactttaatgctgctgacattgttgtttttatgttcatttgctggagcgcaagacgtgctgacacagtgagttttctctgatgtatttaaggtgatgtatttgcttgagttactcctgtatcattgctcataatattggaaactaaaataaaacctagttggaaatcc

SEQ ID NO: 9 - protein, 757 aa, Homo Sapiens
MSLLFSRCNSIVTVKKNKRHMAEVNASPLKHFVTAKKKINGIFEQLGAYIQESATFLEDTYRNAELDPVTTEEQVLDVKGYLSKVRGISEVLARRHMKVAFFGRTSNGKSTVINAMLWDKVLPSGIGHTTNCFLRVEGTDGHEAFLLTEGSEEKRSAKTVNQLAHALHQDKQLHAGSLVSVMWPNSKCPLLKDDLVLMDSPGIDVTTELDSWIDKFCLDADVFVLVANSESTLMQTEKHFFHKVSERLSRPNIFILNNRWDASASEPEYMEEVRRQHMERCTSFLVDELGVVDRSQAGDRIFFVSAKEVLNARIQKAQGMPEGGGALAEGFQVRMFEFQNFERRFEECISQSAVKTKFEQHTVRAKQIAEAVRLIMDSLHMAAREQQVYCEEMREERQDRLKFIDKQLELLAQDYKLRIKQITEEVERQVSTAMAEEIRRLSVLVDDYQMDFHPSPVVLKVYKNELHRHIEEGLGRNMSDRCSTAITNSLQTMQQDMIDGLKPLLPVSVRSQIDMLVPRQCFSLNYDLNCDKLCADFQEDIEFHFSLGWTMLVNRFLGPKNSRRALMGYNDQVQRPIPLTPANPSMPPLPQGSLTQEEFMVSMVTGLASLTSRTSMGILVVGGVVWKAVGWRLIALSFGLYGLLYVYERLTWTTKAKERAFKRQFVEHASEKLQLVISYTGSNCSHQVQQELSGTFAHLCQQVDVTRENLEQEIAAMNKKIEVLDSLQSKAKLLRNKAGWLDSELNMFTHQYLQPSR

SEQ ID NO: 10 - protein, 757 aa, Homo Sapiens; identical to SEQ ID NO: 9

SEQ ID NO: 11 - RNA, 76 nt, Homo Sapiens
gacuauaagcugcgaauuacaugaggccuuucuccuuagcaacucuaucgucacagugguggacgauuaccagaug

SEQ ID NO: 12 - RNA, 76 nt, Homo Sapiens
cgaugaaguaaacgccuuacaugauaggaggaaacgaacagaauauauggaagacguggaaguucuuagugcuaga

SEQ ID NO: 13 - DNA, 20 bp, Artificial Sequence
  Synthetic
tgttgttgtgacgattgctc

SEQ ID NO: 14 - DNA, 20 bp, Artificial Sequence
  Synthetic
ttacgggactagaaaatcag

SEQ ID NO: 15 - DNA, 20 bp, Artificial Sequence
  Synthetic
tgctgtgacatttaagcggt

SEQ ID NO: 16 - DNA, 20 bp, Artificial Sequence
  Synthetic
cgaatgctgtgacatttaag

SEQ ID NO: 17 - DNA, 19 bp, Artificial Sequence
  Synthetic
tttataaaatgcaataggt

(19) United States
(12) Patent Application Publication
(10) Pub. No.: US 2023/0374518 A1
(43) Pub. Date: Nov. 23, 2023

(54) MARF/MFN MODULATORS AND USES THEREOF

(71) Applicant: University of Massachusetts, Boston, MA (US)

(72) Inventors: Eric H. Baeherecke, West Boylston, MA (US); James L. Shen, Shrewsbury, MA (US)

(21) Appl. No.: 18/247,157

(22) PCT Filed: Oct. 1, 2021
(86) PCT No.: PCT/US21/53116

§ 371 Date: Mar. 29, 2023

Related U.S. Application Data
(60) Provisional application No. 63/182,508, filed on Apr. 30, 2021.
(60) Provisional application No. 63/087,111, filed on Oct. 2, 2020.

(51) Int. Cl. C12N 15/113; C12Q 1/34; C12N 9/22; C12N 15/11

(52) U.S. Cl. CPC C12N 15/1137; C12Q 1/34; C12N 9/22; C12N 15/111; C12N 2310/14; C12N 2310/20

(57) ABSTRACT

Aspects of the disclosure relate to compositions and methods for modulating (e.g., inhibiting or promoting) expression of certain mitochondrial regulatory proteins, for example Marf and mitofusin (Mfn). The disclosure is based, in part, on modulation of Marf or Mfn to regulate expression or activity of certain proteins involved in autophagy, for example Vmp1 and vps13D. In some embodiments, compositions and methods described by the disclosure are useful for treating diseases related to aberrant autophagy or mitochondrial function, such as familial neurological movement disorders.

### RELATED APPLICATIONS

This application claims the benefit under 35 U.S.C. 119(e) of the filing date of U.S. provisional Application Ser. No. 63/087,111, filed Oct. 2, 2020, entitled “MARF/MFN MODULATORS AND USES THEREOF”, and U.S. provisional Application Ser. No. 63/182,508, filed Apr. 30, 2021, entitled “MARF/MFN MODULATORS AND USES THEREOF”, the entire contents of each of which are incorporated herein by reference.

### FEDERALLY SPONSORED

This invention was made with government support under GM131689 and CA239374 awarded by the National Institutes of Health. The government has certain rights in the invention.

### BACKGROUND

Autophagy, the lysosome-dependent clearance of intracellular contents, plays important roles in organism development and health. The failure to remove mitochondria by autophagy, or mitophagy, results in defects in cellular homeostasis and health, and contributes to multiple diseases. For example, mutations in genes responsible for mitophagy manifest as inheritable forms of Parkinson's disease and Alzheimer's disease. As a result, understanding the mechanisms that regulate mitophagy under different cellular contexts is crucial to the understanding of biology and health.

### SUMMARY

Aspects of the disclosure relate to compositions and methods for modulating (e.g., inhibiting or promoting) expression of certain mitochondrial regulatory proteins, for example Marf and mitofusin (Mfn). The disclosure is based, in part, on modulation of Marf or Mfn to regulate expression or activity of certain proteins involved in autophagy, for example Vmp1 and vps13D. In some aspects, the disclosure relates to modulation of pink1 to regulate expression or activity of certain proteins involved in autophagy, for example vps13D. In some embodiments, compositions and methods described by the disclosure are useful for treating diseases related to aberrant autophagy or mitochondrial function, such as familial neurological movement disorders.

Accordingly, in some aspects, the disclosure provides a method for treating a disease associated with mitochondrial dysfunction in a subject in need thereof, the method comprising administering to the subject a therapeutically effective amount of a mitofusin (Mfn) modulator.

In some embodiments, a Mfn modulator increases expression or activity of a mitofusin. In some embodiments, a Mfn modulator inhibits expression or activity of a mitofusin. In some embodiments, a mitofusin is Mitofusin 2 (Mfn2).

In some embodiments, a Mfn modulator directly modulates expression or activity of a mitofusin. In some embodiments, a Mfn modulator selectively inhibits expression or activity of a mitofusin. In some embodiments, a Mfn modulator indirectly modulates expression or activity of a mitofusin.

In some embodiments, a Mfn modulator is a nucleic acid, polypeptide, or small molecule. In some embodiments, a nucleic acid is an interfering nucleic acid selected from the group consisting of double stranded RNA (dsRNA), siRNA, shRNA, miRNA, and antisense oligonucleotide (ASO).

In some embodiments, a polypeptide comprises an RNA-guided nuclease. In some embodiments, an RNA-guided nuclease comprises a CRISPR-Cas nuclease.

In some embodiments, a small molecule comprises a proteolysis targeting chimera (PROTAC), a kinase modulator, or an E3 ubiquitin ligase modulator. In some embodiments, a kinase modulator comprises a PINK1 modulator. In some embodiments, a E3 ubiquitin ligase modulator comprises a Mule ligase modulator or Parkin modulator.

In some embodiments, a subject is a mammal. In some embodiments, a subject is a human. In some embodiments, a subject comprises one or more mutations in vps13d. In some embodiments, one or more mutations comprise a frameshift, missense, or partial duplication mutation. In some embodiments, a disease associated with mitochondrial dysfunction is a neurological movement disorder.

In some embodiments, a Mfn modulator is delivered to a neuron of the subject. In some embodiments, a therapeutically effective amount of a Mfn modulator restores normal mitochondrial function in the subject.

In some aspects, the disclosure provides a method for identifying a subject as having a VPS13D-associated disease, the method comprising detecting in a biological sample obtained from a subject an increased level of mitofusin (Mfn) expression or activity relative to a control sample.

In some embodiments, a subject is a mammal. In some embodiments, a subject is a human. In some embodiments, a mitofusin is Mitofusin 2 (Mfn2). In some embodiments, a subject comprises one or more mutations in vps13d.

In some embodiments, methods described herein further comprise administering to the subject one or more Mfn modulators.

In some aspects, the disclosure provides a method for modulating expression or activity of vps13D in a cell (e.g., in a cell of a subject), the method comprising administering to the subject a therapeutically effective amount of a Pink1 modulator.

In some embodiments, the cell is a mammalian cell. In some embodiments, the cell is in a subject. In some embodiments, the subject is a human subject. In some embodiments, the cell or subject comprises one or more mutations in a vps13D gene.

### BRIEF DESCRIPTION OF DRAWINGS

FIGS. 1A-1F show Vmp1 is required for autophagy in Drosophila intestines. FIG. 1A shows vmp1 RNAi intestine cells 2 hours after pupation exhibit decreased mCherryAtg8a puncta formation compared to neighboring control cells. FIG. 1B shows quantification of mCherryAtg8a puncta in vmp1 RNAi cells (n=8) compared to control cells (n=16). FIG. 1C shows vmp1(Δ) mutant cells possess increased Ref2p/p62 puncta compared to neighboring control cells in intestines 2 hours after pupation. FIG. 1D shows quantification of Ref2p puncta in vmp1(Δ) mutant (n=9) and control cells (n=8) 2 hours after pupation. FIG. 1E shows vmp1(Δ) loss-of-function mutant cells possess elevated mitochondrial ATP5a puncta compared to neighboring control cells in intestines 2 hours after pupation. FIG. 1F shows quantification of ATP5a puncta in vmp1(Δ) mutant (n=6) and control cells (n=16) 2 hours after pupation. Scales bars in (A), (C) and (E) represent 40 μm. Error bars in (B), (D) and (F) are SEM. Representative of 3 or more independent biological experiments.

FIGS. 2A-2D show Vmp1 is required for mitophagy and normal mitochondrial morphology in Drosophila intestines. FIG. 2A shows Mito-QC was expressed in different genotypes and analyzed in intestine cells 2 hours after pupation. Control luciferase (luc) RNAi-expressing cells possessed mostly red puncta (reflecting mitochondria in autolysomes, mitolysosomes), while intestine cells expressing RNAi against either vps13d or 2 distinct vmp1 RNAi expressing constructs (#46667 and #100745) all exhibited yellow puncta, reflecting mitochondria that failed to get cleared by mitophagy. FIG. 2B shows quantification of the percentage of mitolysosomes to total mitochondria puncta in luc (n=10) RNAi-, vps13d (n=10) RNAi-, vmp1 (#46667) (n=10) RNAi-, and vmp1 (#100745) (n=8) RNAi-expressing cells 2 hours after pupation. FIG. 2C shows TEM images of cells from intestines expressing either control luciferase (luc) RNAi or vmp1 RNAi 2 hours after pupation. Enlarged regions are outlined by a black box. FIG. 2S shows quantification of the size of mitochondria in either control luc (n=53) RNAi- or vmp1 (n=51) RNAi-expressing intestine cells 2 hours after pupation. Scales bars in (A) represent 40 μm. Scale bars in (C) represent 2.0 μm. Error bars in (B) and (D) are SEM. Representative of 3 or more independent biological experiments.

FIGS. 3A-3F show Vmp1 and Vps13D regulate mitochondria and ER contact. FIG. 3A shows TEM images of cells from intestines expressing either control luciferase (luc) RNAi or vmp1 RNAi 2 hours after pupation. Enlarged regions are outlined by a black box. Mitochondria (M) and ER (arrows) are indicated. FIG. 3B shows quantification of mitochondria and ER contact in either control luc (n=100) RNAi- or vmp1 (n=78) RNAi-expressing intestine cells 2 hours after pupation. Contact between the mitochondria and ER is defined as a distance of less than 0.03 μm and contact length of at least 0.02 μm (16). FIG. 3C shows TEM images of cells from either control +/vps13d (ΔUBA), vps13d (ΔUBA)/vps13d (ΔUBA), or vps13d (ΔUBA)/Df intestines 2 hours after pupation. FIG. 3D shows quantification of mitochondria and ER contact in either control +/vps13d (ΔUBA) (n=50), vps13d (ΔUBA)/vps13d (ΔUBA) (n=50), or vps13d (ΔUBA)/Df (n=50) intestines 2 hours after pupation. FIG. 3E shows TEM images of either wild-type control, VPS13D (ΔUBA), or VPS13D KO (exon 3 deletion) HeLa cells. FIG. 3F shows quantification of mitochondria and ER contact in either control (n=96), VPS13D (ΔUBA) (n=116), or VPS13D KO (exon 3 deletion) (n=100) HeLa cells. In (A), (C), and (E), arrows represent regions of contact between mitochondria (M) and ER. Scale bars in top panels represent 0.5 μm and bottom panels represent 0.03 μm. Error bars in (B), (D), and (F) are SEM. Representative of 3 or more independent biological experiments.

FIGS. 4A-4D show fibroblasts derived from patients with neurological symptoms associated with VPS13D mutations have increased mitochondria and ER contact FIG. 4A shows TEM images of fibroblast cells derived from a family with mutations in VPS13D (Family 1). Cells were derived from either an unrelated donor without mutations in VPS13D (+/+), a relative carrying the G1190D allele for VPS13D (G1190D/+), or a patient with neurological symptoms carrying the G1190D and Q1106* mutations in VPS13D (G1190D/Q1106*). Enlarged regions are outlined by a black box, mitochondria (M) and ER (arrows) are indicated. FIG. 4B shows quantification of mitochondria and ER contact in VPS13D (+/+) (n=54), (G1190D/+) (n=50), and (G1190D/Q1106*) (n=50) fibroblasts derived from Family 1. FIG. 4C shows TEM images of fibroblast cells derived from a family with mutations in VPS13D (Family 2). Cells were derived from either an unrelated donor without mutations in VPS13D (+/+), a relative carrying the A4210V allele for VPS13D (A4210V/+), or the patient with neurological symptoms carrying the A4210V and Y1803* mutations in VPS13D (A4210V and Y1803*). FIG. 4D shows quantification of mitochondria and ER contact in VPS13D (+/+) (n=50), (A4210V/+) (n=56), and (A4210V/Y1803*) (n=50) fibroblasts derived from Family 2. In (A) and (C), scale bars in top panels are 0.5 μm and in bottom panels are 0.03 μm. Error bars in (B) and (D) are SEM. Representative of 3 or more independent biological experiments.

FIGS. 5A-5D show Vps13D puncta formation depends on Vmp1. FIG. 5A shows vmp1 (Δ) loss-of-function mutant cells possess fewer Vps13D puncta compared to neighboring control cells in intestines 2 hours after pupation. FIG. 5B shows quantification of Vps13D puncta in vmp1(Δ) mutant (n=6) and control (n=16) intestine cells 2 hours after pupation. FIG. 5C shows vps13d (MiMic) mutant cells (lacking nuclear RFP) do not have altered GFP-Vmp1 in larval intestines 2 hours after pupation. Antibody against GFP was used to enhance GFP-Vmp1 signal. FIG. 5D shows quantification of GFP-Vmp1 puncta in vps13d (MiMIc) mutant (n=8) and control (n=14) intestine cells 2 hours after pupation. Scale bars in (A) and (C) represent 40 μm. Error bars in (B) and (D) are SEM. Representative of 3 or more independent biological experiments.

FIGS. 6A-6F show Vps13d and Vmp1 function in a pathway to regulate mitophagy and mitochondrial morphology. FIG. 6A shows vmp1(Δ) and vps13d (ΔUBA) double mutant cells exhibit similar levels of mitochondrial ATP5a protein compared to neighboring control vmp1(Δ)/+ and vps13d (ΔUBA) single mutant cells 2 hours after pupation. FIG. 6B shows quantification of ATP5a puncta in vmp1(Δ) and vps13d (ΔUBA) double mutant (n=8) and vmp1(Δ)/+ and vps13d (ΔUBA) single mutant (n=14) control intestine cells 2 hours after pupation. FIG. 6C shows Mito-QC was expressed in different genotypes and analyzed in intestine cells 2 hours after pupation. Control vps13d (ΔUBA)/+cells possessed mostly red puncta (reflecting mitochondria in autolysomes, mitolysosomes), while vps13d (ΔUBA/ΔUBA) homozygous mutant, vps13d (ΔUBA/ΔUBA) mutant expressing flp, and vps13d (ΔUBA/ΔUBA) mutant with vmp1 RNAi-expressing intestine cells all exhibited large yellow puncta (reflecting mitochondria that fail to be cleared by mitophagy). FIG. 6D shows quantification of the percentage of mitolysosomes to total mitochondria puncta in vps13d (ΔUBA)/+(n=10), vps13d (ΔUBA/ΔUBA) (n=10), vps13d (ΔUBA/ΔUBA), uas-flp (n=11), and vps13d (ΔUBA/ΔUBA), uas-vmp1-IR (n=10), cells 2 hours after pupation. FIG. 6E shows TEM images of cells from either control vps13d (ΔUBA)/MiMic expressing rfp RNAi or vps13d (ΔUBA)/MiMic expressing vmpl RNAi intestines 2 hours after pupation. Enlarged regions are outlined by a black box, mitochondria (M) and ER (arrows) are indicated. FIG. 6F shows quantification of either mitochondrial size or mitochondria and ER contact in either control vps13d (ΔUBA)/MiMic expressing rfp RNAi (n=55) or vps13d (ΔUBA)/MiMic expressing vmpl RNAi (n=62) intestine cells 2 hours after pupation. Scale bars in (A) and (C) are 40 μm. Scale bars in top panel of (E) represent 0.5 μm while scale bars in bottom panels represent 0.03 μm. Error bars in (B), (D), and (F) are SEM. Representative of 3 or more independent biological experiments.

FIGS. 7A-7H show Vps13d and Marf physically interact to mediate mitochondrial clearance and mitochondria and ER contact sites. FIG. 7A shows a Western blot of input and eluates from a FLAG immunoprecipitation of control w1118 and vps13d-3xflag pupae 2 hours after pupation that was probed with antibodies against Marf and Actin. Vps13D-3xFLAG levels were too low to be detected in the input with lysate conditions suitable for immunoprecipitation, so the presence of 3xFLAG tagged Vps13D was verified using harsher lysis conditions. FIG. 7B shows a Western blot of lysates from vps13d(ΔUBA)/+, Df/+, and vps13d(ΔUBA)/Df intestines 2 hours after pupation that was probed with antibodies against Marf, ATP5a and Actin. FIG. 7C shows quantification of relative levels of Marf and ATP5a in vps13d(ΔUBA)/+, Df/+, and vps13d(ΔUBA)/Df intestines 2 hours after pupation compared to Actin. FIG. 7D shows intestine cells that overexpress marf using the Act-GAL4 were stained with antibodies against ATP5a and compared to neighboring control cells. FIG. 7E shows quantification of levels of ATP5a puncta in marf overexpressing intestine cells 2 hours after pupation compared to control cells. FIG. 7F shows TEM images of cells from either control vps13d (ΔUBA)/MiMic expressing rfp RNAi (left panels) or vps13d (ΔUBA)/MiMic expressing marf RNAi (right panels) intestines 2 hours after pupation. Enlarged regions are outlined by a black box, mitochondria (M) and ER (arrows) are indicated. FIG. 7G shows quantification of mitochondrial size in either control vps13d (ΔUBA)/MiMic expressing rfp RNAi (n=84) or vps13d (ΔUBA)/MiMic expressing marf RNAi (n=74) intestine cells 2 hours after pupation. FIG. 7H shows quantification mitochondria and ER contact in either control vps13d (ΔUBA)/MiMic expressing rfp RNAi (n=84) or vps13d (ΔUBA)/MiMic expressing marf RNAi (n=74) intestine cells 2 hours after pupation. Scale bar in (D) represents 40 μm. Scale bars in left panels of (F) represent 0.5 μm while scale bars in right panels represent 0.03 μm. Error bars in (C), (E), and (G) and (H) are SEM. Representative of 3 or more independent biological experiments.

FIGS. 8A-8G show VPS13D is essential in human cell lines and vmp1 and vps13d mutants share autophagy deficiencies in developing Drosophila intestines. FIG. 8A shows Genetic Dependency Data from the CRISPR Cancer Dependency Map was compiled for essential genes, tumor suppressors, VPS13D, and other Vps13 family members. Essential genes mechanistic target of rapamycin (MTOR) and DNA polymerase alpha catalytic subunit (POLA1) received Achilles Scores near −1, indicating strong dependency for survival in cultured cell lines. Tumor suppressors tumor protein p53 (TP53) and phosphatase and tensin homolog (PTEN), which may result in enhanced cell survival in cultured cell lines when lost, received positive scores indicating non-essentiality. While vacuolar protein sorting 13A-C (VPS13A-C) scores indicate that they are not required for survival, vacuolar protein sorting 13 d (VPS13D) is similar to other essential genes and is required for survival. FIG. 8B shows vmp1 RNAi (VDRC line #46667) intestine cells exhibit decreased mCherryATG8a puncta formation compared to neighboring control cells 2 hours after pupation. FIG. 8C shows quantification of mCherryATG8a puncta in vmpl (n=6) RNAi (#46667)-expressing and control (n=11) intestine cells 2 hours after pupation. FIG. 8D shows design of the Drosophila vmp1(Δ) mutant using sgRNA1 and sgRNA2. Numbers represent amino acid sequence. FIG. 8E shows number of surviving vmp1(Δ) loss of function mutant and vmp1(+) control adult male flies with or without a duplication of the genomic region that contains vmp1. Results compiled from 100 eclosed male flies. p value derived from the Fisher Exact Test. FIG. 8F shows vps13d (MiMic) loss of function intestine cells were stained with antibody against Ref2p and compared with neighboring vps13d (MiMic)/+control cells. FIG. 8G shows quantification of Ref2p puncta in vps13d (MiMic) loss of function intestine cells (n=7) compared to vps13d (MiMic)/+control intestine cells (n=12). Scale bars in (B) and (F) represent 40 μm. Error bars in (A) (C) and (G) represent SEM. Representative of 3 or more independent biological experiments.

FIG. 9 shows vmp1 RNAi-expressing larval intestine cells have increased mitochondria size 2 hours after pupation. Quantification of mitochondria area in TEM sections of either control luc (n=53) RNAi- or vmp1 (n=50) RNAi (VDRC line #46667)-expressing intestine cells 2 hours after pupation. Error bars represent SEM. Representative of 3 or more independent biological experiments.

FIG. 10 shows vmp1 RNAi-expressing larval intestine cells have increased mitochondria and ER contact 2 hours after pupation. The percentage of contact between mitochondria and ER in TEM sections of either control luc RNAi (n=53) or vmp1 RNAi (VDRC line #46667)-expressing intestine cells 2 hours after pupation. Error bars represent SEM. Representative of 3 or more independent biological experiments.

FIGS. 11A-11E show genetic interaction analyses between vmp1 and vps13d in Drosophila and C. elegans, and characterization of gfp-vmp1. FIG. 11A shows vmp1(Δ)/FM7i-pAct-GFP, vps13d(MiMic)/TM6B-vDAa and vmp1(Δ)/FM7i-pAct-GFP;vps13d(MiMic)/TM6B-vDAa virgin female flies were crossed with control (w1118) males and allowed to lay eggs on standard media. Progeny were allowed to reach adulthood and genotypes of eclosed adult flies were determined. Fewer vmp1(Δ)/+ and vps13d(MiMic)/+flies eclosed than controls of either vmp1(Δ)/+ or vps13d(MiMic)/+flies, which had percentages close to the normal expected Mendelian distribution of 50%. Data compiled from (3) 48-hour egg lays. Only female adult flies were scored due to vmp1(Δ) being on the X chromosome and being homozygous lethal. FIG. 11B shows wild-type or vmp1/epg3 mutant (bp933) background C. elegans worms were fed empty vector control (−) or vps13d (+) RNAi. Parental generation (P0) vmp1 mutant worms fed vps13d RNAi experienced about a 25% reduction in fertility and all first filial generation (F1) worms were inviable. Worms in all other conditions were unaffected. Representative of 3 or more independent biological experiments. FIG. 11C shows design of the Drosophila gfp-vmp1 using sgRNA3 and sgRNA4. Numbers represent amino acid sequence. FIG. 11D shows gfp-vmp1 intestine cells were dissected from early 3rd instar larvae (top) and 2 hours after pupation (bottom). Early 3rd instar larvae expressed tdTomato-Sec61β while intestines 2 hours after pupation were stained with antibody against SERCA, both of which are associated with ER. GFP specific antibody was used to enhance gfp-vmp1 signal. GFP-Vmp1 colocalized with the ER at both stages. FIG. 11E shows gfp-vmp1 larval intestine cells have distinct gfp-vmp1 puncta, are able to clear most mitochondrial ATP5a protein and have reduced cell size (dotted line) in intestines that express control luc RNAi 2 hours after pupation (top). By contrast, animals that express vmp1 RNAi in intestines (bottom) possess depleted gfp-vmp1, retained mitochondria ATP5a protein, and enlarged cells (dotted line). Antibody against GFP was used to enhance gfp-vmp1 signal. Scale bars in enlarged images in (D) (bordered with dotted lines) represent 8 μm. All other scale bars represent 40 μm. Representative of 3 or more independent biological experiments.

FIG. 12 shows loss of vps13d fails to enhance the mitochondrial clearance deficiency in larval intestines cells with reduced vmp1 function. vps13d(MiMic) mutant cells (non-GFP, dotted line) in intestines that express vmp1 RNAi in all cells and stained for mitochondrial ATP5a protein 2 hours after pupation. Scale bars represent 40 μm. Representative of 3 or more independent biological experiments.

FIGS. 13A-13H show vps13d-3xflag flies have normal mitochondrial morphology and clearance, and Marf does not affect Vps13D puncta formation. FIG. 13A shows design of the Drosophila vps13d-3xflag using sgRNA5. Numbers represent amino acid sequence. FIG. 13B shows intestines dissected from control w1118 and vps13d-3xflag animals 2 hours after pupation were stained with antibody against ATP5a. FIG. 13C shows intestines dissected from vps13d-3xflag animals 2 hours after pupation were stained with antibodies against Vps13D and FLAG. FIG. 13D shows lysates from control w1118 and vps13d-3xflag 2 hours after pupation were analyzed by western Blot with antibodies against FLAG and Actin. FIG. 13E shows 2-hour pupal lysates from either control luc or marf RNAi driven by the Act-ga14 driver were analyzed by western Blot with antibodies against Marf and ATP5a. FIG. 13F shows intestines dissected from animals 2 hours after pupation expressing either control luc RNAi or marf RNAi driven by the NP1-GAL4 driver, and expressing UAS-mito-GFP, were compared by immunofluorescence. FIG. 13G shows intestines 2 hours after pupation containing marf(B) loss-of-function mutant cells (non-RFP) were stained with antibody against ATP5a (purple). FIG. 13H shows intestines 2 hours after pupation containing marf(B) loss-of-function mutant cells (non-RFP) were stained with antibody against Vps13D. Scale bars in (B), (C), (F), (G), and (H) represent 40 μm. Representative of 3 or more independent biological experiments.

FIGS. 14A-14H show reduction of Marf/Mfn2 function suppresses Vps13D and Vmp1 phenotypes. A) vps13d (ΔUBA/MiMic) and vmp1 RNAi-expressing intestines 2 hours after pupariation were stained with antibody against ATP5a (purple) with control rfp RNAi or marf RNAi expression. (B) Quantification of ATP5a puncta size in vps13d (ΔUBA/MiMic) and vmp1 RNAi-expressing intestines 2 hours after pupariation with control rfp RNAi (n=13 for vps13d, n=11 for vmp1) or marf RNAi (n=12 for vps13d and vmp1) expression. (C) Fibroblasts from a patient (mutant) with trans heterozygous VPS13D mutations (G1190D/Q1106*) were stained with TOMM20 antibody (green) and compared to heterozygous control (G1190D/+) fibroblasts (control). Control fibroblasts were transfected with negative control mock and VMP1 RNAi and mutant fibroblasts were transfected with mock, MFN2 and VMP1 RNAi. (D) Quantification of mitochondria morphology in control fibroblasts transfected with mock RNAi (n=11) and VMP1 RNAi (n=10) compared to mutant fibroblasts transfected with mock RNAi (n=11), VMP1 RNAi (n=15), and MFN2 RNAi (n=14). (E) Representative TEM images of cells from vps13d (ΔUBA)/(MiMic) intestine cells expressing either rfp (control) or marf RNAi (left panels) 2 hours after pupariation, and VPS13D (A4210V/Y1803*) patient fibroblasts treated with either negative control mock or MFN2 RNAi (right panels). Enlarged regions are outlined by a black box, mitochondria (M) and ER (arrows) are indicated. (F) Quantification of mitochondrial size in vps13d (MiMic)/+(n=62) intestine cell expressing rfp RNAi, vps13d (MiMic)/+(n=82) intestine cells expressing marf RNAi, vps13d (ΔUBA)/(MiMic) (n=84) intestine cell expressing rfp RNAi, and vps13d (ΔUBA)/(MiMic) (n=72) intestine cells expressing marf RNAi 2 hours after pupariation. (G) Quantification mitochondria and ER contact in vps13d (MiMic)/+(n=62) intestine cells expressing rfp RNAi, vps13d (MiMic)/+(n=82) intestine cells expressing marf RNAi, vps13d (ΔUBA)/(MiMic) (n=84) intestine cells expressing rfp RNAi, and vps13d (ΔUBA)/(MiMic) (n=72) intestine cells expressing marf RNAi 2 hours after pupariation. (H) Quantification of mitochondria and ER contact in VPS13D (A4210/+) heterozygous control fibroblasts treated with mock (n=50) and MFN2 (n=51) RNAi compared to VPS13D (A4210V/Y1803*) mutant fibroblasts treated with mock (n=50) and MFN2 (n=50) RNAi. Scale bar in top panels of A) and C) represents 40 μm, bottom panels represent 10 μm. Scale bars in the upper panels of (E) represent 0.5 μm while scale bars in bottom panels represent 0.03 μm. Error bars in (B), (D), (F), (G), and (H) are SEM. Thresholding in (A) and (C) were based on maximizing the quality of signals without over-saturation. Representative of 3 or more independent biological experiments.

FIGS. 15A-15C show marf RNAi partially suppressed vps13d mutant phenotypes, and siRNA knockdown of MFN2 and MFN1 levels in human fibroblasts. (A) vps13d (ΔUBA/MiMic) mutant intestine cells expressing Mito-QC and marf RNAi through the intestine specific NP1-GAL4 driver had less GFP and mCherry positive puncta than vps13d (66 UBA/ΔUBA) mutant intestine cells 2 hours after pupariation (FIG. 5G). (B) vps13d (ΔUBA/MiMic) mutant intestines cells expressing either marf RNAi or FLP through the intestine specific NP1-GAL4 driver were immuno-stained with antibodies against ATP5a (purple) and Ref2p (green) 2 hours after pupariation. marf RNAi expressing mutant intestine cells had less enlarged Ref2p accumulations. (C) Human derived fibroblasts from the UMCtrl1 cell line were transfected with MFN2 and MFN1 siRNA SMARTpool for 48 hours, lysed, and analyzed for MFN2, MFN1, and Actin protein. Representative of 3 or more independent biological experiments.

FIGS. 16A-16H show Vps13D functions in a mitophagy pathway with Pink1. FIG. 16A shows representative TEM images of male pink1^{B9} (−);vps13d (ΔUBA/+) single-mutant and pink1^{B9} (−);vps13d (ΔUBA/ΔUBA) double-mutant intestine cells 2 h after pupariation. FIG. 16B shows quantification of mitochondria area in pink1^{B9} (−);vps13d (ΔUBA/+) single-mutant (n=187) and pink1^{B9};vps13d (ΔUBA/ΔUBA) double-mutant (n=181) intestine cells 2 h after pupariation. FIG. 16C shows quantification of the percentage of mitochondria <0.01 μm and ≥0.1 μm in pink1^{B9} (−);vps13d (ΔUBA/+) single-mutant (n=187)and pink1^{B9} (−);vps13d (ΔUBA/ΔUBA) double-mutant (n=181) intestine cells 2 h after pupariation using Fisher's exact test (P=0.7507). FIG. 16D shows Mito-QC was expressed using the Myo31DFNP0001 driver in pink1^{B9} (−);vps13d (ΔUBA) mutant intestine cells from 2-h-old male prepupae. FIG. 16E shows quantification of the amount of structures that presented as enlarged puncta in pink1 (+);vps13d (ΔUBA)/+ heterozygote control (n=17),pink1^{B9} (−);vps13d (ΔUBA)/+ single-mutant (n=14),pink1 (+);vps13d (ΔUBA/ΔUBA) single-mutant, and pink1^{B9} (−);vps13d (ΔUBA)/ΔUBA) double-mutant (n=14) cells 2 h after pupariation. FIG. 16F shows pink1^{B9}/pink1^{B9} (−/−);vps13d (ΔUBA/ΔUBA) double-mutant intestine cells have similar levels and size of Atg8a puncta compared with pink1^{B9}+ (+/−);vps13d (ΔUBA/ΔUBA) single-mutant cells 2 h after pupariation. FIG. 16G shows quantification of Atg8a puncta number in pink1^{B9}/pink1^{B9} (−/−);vps13d (ΔUBA/ΔUBA) double-mutant cells (n=11) compared with neighboring pink1^{B9}/+ (+/−);vps13d (ΔUBA/ΔUBA) single-mutant cells (n=15). FIG. 16H shows quantification of Atg8a puncta size in pink1^{B9}/pink1^{B9} (−/−);vps13d (ΔUBA/ΔUBA) double-mutant cells (n=11) compared with pink1^{B9}+ (+/−);vps13d (ΔUBA/ΔUBA) single-mutant cells (n=15). Scale bars in A are 0.5 μm. Scale bars in D and F are 40 μm with the exception of the enlarged images in D, which are 5 μm.

FIGS. 17A-17F show loss of Pink1 suppresses ubiquitin localization to mitochondria in vps13d mutant cells. FIG. 17A shows pink1^{B9} (−);vps13d (ΔUBA/+) single-mutant intestine cells have conjugated ubiquitin puncta that do not encircle mitochondria labeled by mito-GFP as frequently as intestines from pink1 (+);vps13d (ΔUBA/ΔUBA) single mutants 2 h after prepupae formation (top panels). Loss of Pink1 in a vps13d (ΔUBA/ΔUBA) background, resulting in a pink1^{B9} (−);vps13d (ΔUBA/ΔUBA) double mutant (bottom panels) suppresses the conjugated ubiquitin localization to mitochondria phenotype in vps13d mutant intestine cells (middle panels) 2 h after pupariation. FIG. 17B shows quantification of the number of mito-GFP puncta with at least 50% of the perimeter encircled by conjugated ubiquitin in pink1^{B9} (−);vps13d (ΔUBA/+) (n=15),pink1 (+);vps13d (ΔUBA/ΔUBA) (n=11),and pink1^{B9} (−);vps13d (ΔUBA/ΔUBA) (n=12) mutant intestines 2 h after pupariation. FIG. 17C shows pink1 (+);vps13d (ΔUBA/ΔUBA) single-mutant intestine cells have Ser65 phosphorylated ubiquitin (pUb) puncta surrounding mitochondria labeled by ATP5a 2 h after puparium formation. pink1^{B9} (−); vps13d (ΔUBA/ΔUBA) double-mutant intestine cells do not have Ser65 pUb puncta surrounding mitochondria 2 h after prepupae formation. FIG. 17D shows quantification of the number of ATP5a puncta with at least 50% of the perimeter encircled by Ser65 pUb in pink1 (+);vps13d (ΔUBA/ΔUBA) single-mutant (n=15) and pink1^{B9};vps13d (ΔUBA/ΔUBA) double-mutant (n=15) intestines 2 h after pupariation. FIG. 17E shows pink1^{B9}/pink1^{B9} (−/−) mutant intestine cells have decreased Vps13D puncta compared with pink1^{B9}/+(+/−) heterozygous control neighboring cells. FIG. 17F shows quantification of Vps13D puncta in pink1^{B9}/pink1^{B9} (−/−) cells (n=6) compared with control (+/−)cells(n=14). Scale bars in A, C, and E are 40 μm with the exception of the enlarged images in A and C, which are 5 μm.

### DETAILED DESCRIPTION

Aspects of the disclosure relate to compositions and methods for modulating (e.g., inhibiting or promoting) expression of certain mitochondrial regulatory proteins, for example Marf and mitofusin (Mfn) and/or PTEN-induced kinase 1 (PINK1). The disclosure is based, in part, on modulation of Marf or Mfn and/or PINK1 to regulate expression or activity of certain proteins involved in autophagy, for example Vmp1 and vps13D. In some embodiments, compositions and methods described by the disclosure are useful for treating diseases related to aberrant autophagy or mitochondrial function, such as familial neurological movement disorders.

### Mitochondrial Dysfunction

In some aspects, the disclosure relates to compositions and methods for treating a subject having or suspected of having a disease associated with mitochondrial dysfunction. As used herein, a “subject” is interchangeable with a “subject in need thereof”, both of which may refer to a subject having a disease associated with mitochondrial dysfunction, or a subject having an increased risk of developing such a disease relative to the population at large. A subject in need thereof may be a subject having a mitochondrion that exhibits aberrant activity, or a subject having one or more mutations in a gene that results in aberrant mitochondrial function (e.g., vps13d). A subject can be a human, non-human primate, rat, mouse, cat, dog, or other mammal. In some embodiments, a subject is a human. In some embodiments, a subject is an invertebrate, for example a fly (e.g., Drosophila) or a nematode (e.g., C. elegans).

As used herein, a “disease associated with mitochondrial dysfunction” refers to a disease characterized by aberrant (e.g., reduced, relative to a healthy individual) mitochondrial activity. In some embodiments, a disease associated with mitochondrial dysfunction is caused by a loss of efficiency in the electron transport chain and reductions in the synthesis of high-energy molecules, such as adenosine-5′-triphosphate (ATP). In some embodiments, a subject having a disease associated with mitochondrial dysfunction is also characterized by aberrant endoplasmic reticulum (ER) contact and/or dysregulated autophagy. Examples of diseases associated with mitochondrial dysfunction include but are not limited to familial neurological movement disorders (e.g., ataxia, dystonia, chorea, VPS13D motor diseases, etc.), Alzheimer's disease, Parkinson's disease, Huntington's disease, amyotrophic lateral sclerosis, Friedreich's ataxia, cardiovascular diseases, such as atherosclerosis and other heart and vascular conditions, diabetes and metabolic syndrome, autoimmune diseases, such as multiple sclerosis, systemic lupus erythematosus, and type 1 diabetes, neurobehavioral and psychiatric diseases, such as autism spectrum disorders, schizophrenia, and bipolar and mood disorders, gastrointestinal disorders, fatiguing illnesses, such as chronic fatigue syndrome and Gulf War illnesses, musculoskeletal diseases, such as fibromyalgia and skeletal muscle hypertrophy/atrophy, cancer, and chronic infections.

In some embodiments, a subject “having” or “suspected of having” a disease associated with mitochondrial dysfunction can be a subject that is known or determined to have one or more mutations in a gene associated with mitochondrial function (e.g., vps13d, PINK1, Mule, etc.), or a subject exhibiting signs and symptoms of a disease associated with mitochondrial dysfunction, including but not limited to motor dysfunction (e.g., spasticity, ataxia, chorea, dystonia), muscle atrophy, ocular dysfunction (e.g., blurry vision, trouble focusing, etc.), heart disease, kidney disease, liver disease, thyroid dysfunction, and/or neuropsychiatric manifestations (e.g., compulsive behavior, apathy, anxiety, etc.).

A subject having or suspected of having a disease associated with mitochondrial dysfunction may comprise one or more mutations in a vps13d gene. Vacuolar Protein Sorting 13 Homolog D (VPS13D) is a protein involved in trafficking of membrane proteins between the trans-Golgi network and the pre-vacuolar compartment. In humans, VPS13D is encoded by the vps13d gene, for example as set forth in NCBI Reference Sequence Accession Number NM_015378.4 (SEQ ID NO: 1) and NM_018156.4 (SEQ ID NO: 2). In some embodiments, VPS13D protein comprises the sequence set forth in NCBI Reference Sequence Accession Number NP_056193.2 (SEQ ID NO: 3) or NP_060626.2 (SEQ ID NO: 4). Examples of mutations in vps13d are known in the art and are described for example by Gauthier et al. (2018) Ann Neurol 83, 1089-1095; and Seong et al. (2018) Ann Neurol 83, 1075-1088.

As used herein, the terms “treatment”, “treating”, and “therapy” refer to therapeutic treatment and prophylactic or preventative manipulations. The terms further include ameliorating existing symptoms, preventing additional symptoms, ameliorating or preventing the underlying causes of symptoms, preventing or reversing causes of symptoms, for example, symptoms associated with a disease associated with mitochondrial dysfunction (e.g., a VPS13D neurological movement disease). Thus, the terms denote that a beneficial result has been conferred on a subject with a disorder (e.g., a disease associated with mitochondrial dysfunction), or with the potential to develop such a disorder. Furthermore, the term “treatment” is defined as the application or administration of an agent (e.g., therapeutic agent or a therapeutic composition) to a subject, or an isolated tissue or cell line from a subject, who may have a disease, a symptom of disease or a predisposition toward a disease, with the purpose to cure, heal, alleviate, relieve, alter, remedy, ameliorate, improve or affect the disease, the symptoms of disease or the predisposition toward disease.

Therapeutic agents or therapeutic compositions may include a compound in a pharmaceutically acceptable form that prevents and/or reduces the symptoms of a particular disease (e.g., a disease associated with mitochondrial dysfunction, such as a VPS13D neurological movement disease). For example, a therapeutic composition may be a pharmaceutical composition that prevents and/or reduces the symptoms of a disease associated with mitochondrial dysfunction. It is contemplated that the therapeutic composition of the present invention will be provided in any suitable form. The form of the therapeutic composition will depend on a number of factors, including the mode of administration as described herein. The therapeutic composition may contain diluents, adjuvants and excipients, among other ingredients as described herein.

### Mitofusin (Mfn) Modulators

Aspects of the disclosure relate to methods of treating certain diseases or disorders (e.g., diseases associated with mitochondrial dysfunction, such as VPS13D-associated diseases) that comprise administering a therapeutically effective amount of a mitofusin (Mfn) modulator to a subject. Mitofusins are GTPase enzymes embedded in the outer membrane of the mitochondria. Humans have two mitofusins, Mfn1 and Mfn2. In some embodiments, Mfn1 is encoded by NCBI Reference Sequence Accession Number NM_033540 (SEQ ID NO: 5). In some embodiments, Mfn1 comprises the amino acid sequence set forth in NCBI Reference Sequence Accession Number NP_284941 (SEQ ID NO: 6). In some embodiments, Mfn2 is encoded by NCBI Reference Sequence Accession Number NM_001127660 (SEQ ID NO: 7) or NM_014874 (SEQ ID NO: 8). In some embodiments, Mfn2 comprises the amino acid sequence set forth in NCBI Reference Sequence Accession Number NP_001121132 (SEQ ID NO: 9) or NP_055689 (SEQ ID NO: 10). In some embodiments, a gene encoding a mitofusin (e.g., Mfn1, Mfn2, etc.) is homologous to a marf gene in Drosophila.

A “modulator” refers to an agent that alters the transcriptional activity of a target gene, for example vps13D or mitofusin (e.g., Mfn1, Mfn2, etc.) or pink1. For example, in some embodiments a modulator of Mfn (e.g., Mfn1, Mfn2, etc.) increases the transcriptional activity of Mfn1 and/or Mfn2. in some embodiments a modulator of PINK1 increases the transcriptional activity of pink1 (e.g., human pink1). Increased transcriptional activity generally results in increased production of mRNA and/or increased protein translation (e.g., translation of Mfn1 and/or Mfn2 protein, translation of PINK1, etc.). In some embodiments a modulator of Mfn (e.g., Mfn1, Mfn2, etc.) decreases (e.g., inhibits) the transcriptional activity of Mfn1 and/or Mfn2. In some embodiments a modulator of Pink1 decreases (e.g., inhibits) the transcriptional activity of pink1. Decreased transcriptional activity generally results in decreased production of mRNA and/or decreased protein translation (e.g., translation of Mfn1 and/or Mfn2 protein, translation of PINK1 protein). A modulator can directly alter transcriptional activity of an Mfn (e.g., Mfn1 and/or Mfn2) or PINK1, or can indirectly alter Mfn (e.g., Mfn1 and/or Mfn2) or PINK1 transcriptional activity by interacting with another factor (e.g., protein) that modulates expression and/or the epigenetic state of a Mfn gene. In some embodiments, a modulator of Mfn inhibits the expression level or activity (e.g., function) of another protein that modulates transcriptional activity of a Mfn protein. For example, in some embodiments, a modulator of Mfn is an agent that inhibits or promotes phosphorylation (e.g., a kinase inhibitor or promoter) or inhibits or promotes ubiquitination (e.g., an E3 ligase inhibitor or promoter. In some embodiments, a modulator of Mfn can be a nucleic acid, polypeptide, small molecule, or any combination of the foregoing.

In some embodiments, a modulator of Mfn modulates expression or activity of a ubiquitin ligase. As used herein, the term “ubiquitin ligase” refers to an enzyme that recruits an E2 ubiquitin-conjugating enzyme that has been loaded with ubiquitin, recognizes a protein substrate (e.g., a histone protein), and assists or directly catalyzes the transfer of ubiquitin from the E2 to the protein substrate (e.g., histone protein). In some embodiments, the disclosure relates to modulators of E3 ubiquitin ligase enzymes. E3 ubiquitin ligases are generally split into four families (HECT, RING-finger, U-box and PHD-finger). In some embodiments, the disclosure relates to modulators of Mule ligase. Mule ligase is an E3 ligase involved in ubiquitination of mitochondrial proteins, for example VPS13D, and is described for example by Dadson et al. (2017) Scientific Reports volume 7, Article number: 41490. Example of E3 ligase modulators include but are not limited to cyclosporine, FK506, rapamycin, lenalidomide, pomalidomide, thalidomide, PRT4165, Bortezomib, and E3 inhibitors described by Landre et al. Oncotarget. 2014 Sep; 5(18): 7988-8013. In some embodiments, a ubiquitin ligase modulator increases expression or activity of a Mfn (e.g., Mfn1, Mfn2, etc.). In some embodiments, a ubiquitin ligase modulator decreases expression or activity of a Mfn (e.g., Mfn1, Mfn2, etc.).

In some embodiments, a modulator of Mfn modulates expression or activity of a kinase. In some embodiments, the kinase is PINK1, which induces parkin protein to bind to depolarized mitochondria to induce autophagy of those mitochondria. PTEN-induced kinase 1 (PINK1) is a mitochondrial serine/threonine-protein kinase encoded by the PINK1 gene. Examples of modulators of PINK1 include but are not limited to AF-6, parkin, MB-10, DECA, celastrol, etc. In some embodiments, a kinase modulator increases expression or activity of a Mfn (e.g., Mfn1, Mfn2, etc.). In some embodiments, a kinase modulator decreases expression or activity of a Mfn (e.g., Mfn1, Mfn2, etc.).

In some embodiments, a modulator of Mfn is a selective inhibitor. In some embodiments, a modulator of PINK1 is a selective inhibitor. As used herein, a “selective inhibitor” or an inhibitor that is said to “selectively inhibit” refers to an inhibitor that preferentially inhibits activity or expression of a target molecule of a particular class compared with other molecules of the class. In some embodiments, a selective inhibitor of a target molecule of a particular class has half maximal inhibitory concentration (IC₅₀) relative to the target molecule that is at least 2-fold, at least 4-fold, at least 8-fold, at least 10-fold, at least 20-fold, at least 30-fold, at least 40-fold, or at least 50-fold lower than the IC₅₀ relative to one or more other members of the class. A selective inhibitor can be an inhibitor of a mitofusin (e.g., Mfn1, Mfn2, etc.), a ubiquitin ligase (e.g., an E3 ubiquitin ligase), a kinase (e.g., PINK1), etc.

In some embodiments, a modulator of Mfn is an interfering RNA. Examples of interfering RNA include, but are not limited to double stranded RNA (dsRNA), siRNA, shRNA, miRNA, and antisense oligonucleotides (ASOs). Inhibitory oligonucleotides may interfere with gene expression, transcription and/or translation. Generally, inhibitory oligonucleotides bind to a target polynucleotide via a region of complementarity. For example, binding of inhibitory oligonucleotide to a target polynucleotide can trigger RNAi pathway-mediated degradation of the target polynucleotide (in the case of dsRNA, siRNA, shRNA, etc.), or can block the translational machinery (e.g., antisense oligonucleotides). Inhibitory oligonucleotides can be single-stranded or double-stranded. In some embodiments, inhibitory oligonucleotides are DNA or RNA. In some embodiments, the inhibitory oligonucleotide is selected from the group consisting of an antisense oligonucleotide, siRNA, shRNA and miRNA. In some embodiments, inhibitory oligonucleotides are modified nucleic acids.

The term “nucleotide analog” or “altered nucleotide” or “modified nucleotide” refers to a non-standard nucleotide, including non-naturally occurring ribonucleotides or deoxyribonucleotides. In some embodiments, nucleotide analogs are modified at any position so as to alter certain chemical properties of the nucleotide yet retain the ability of the nucleotide analog to perform its intended function. Examples of positions of the nucleotide which may be derivitized include the 5 position, e.g., 5-(2-amino)propyl uridine, 5-bromo uridine, 5-propyne uridine, 5-propenyl uridine, etc.; the 6 position, e.g., 6-(2-amino)propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., 2000 Aug. 10(4):297-310.

Nucleotide analogs may also comprise modifications to the sugar portion of the nucleotides. For example the 2′ OH-group may be replaced by a group selected from H, OR, R, F, Cl, Br, I, SH, SR, NH₂, NHR, NR₂, COOR, or, wherein R is substituted or unsubstituted C.sub.1-C.sub.6 alkyl, alkenyl, alkynyl, aryl, etc. Other possible modifications include those described in U.S. Pat. Nos. 5,858,988, and 6,291,438. A locked nucleic acid (LNA), often referred to as inaccessible RNA, is a modified RNA nucleotide. The ribose moiety of an LNA nucleotide is modified with an extra bridge connecting the 2′ oxygen and 4′ carbon.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) preferably decrease the rate of hydrolysis of, for example, polynucleotides comprising said analogs in vivo or in vitro. In some embodiments, the inhibitory oligonucleotide is a modified inhibitory oligonucleotide. In some embodiments, the modified inhibitory oligonucleotide comprises a locked nucleic acid (LNA), phosphorothioate backbone , and/or a 2′-OMe modification.

In some embodiments, an inhibitory nucleic acid specifically binds to at least 2, 3, 4, 5, 6, 7, 8, 9, 10, 11, 12, 13, 14, 15, 16, 17, 18, 19, 20, 21, 22, 23, 24, 25, or more contiguous nucleotides of a nucleic acid (e.g., an mRNA transcript) encoding a mitofusin, for example Mfn1 (e.g., SEQ ID NO: 5) or Mfn2 (e.g., SEQ ID NO: 7 or 8). In some embodiments, an inhibitory nucleic acid comprises the sequence set forth in SEQ ID NO: 11 or 12. In some embodiments, an inhibitory nucleic acid comprises a sequence encoded by the sequence set forth in SEQ ID NO: 11 or 12.

In some embodiments, a modulator of Mfn comprises a Proteolysis Targeting Chimera (PROTAC). A “PROTAC” generally refers to a heterobifunctional small molecule composed of two active domains and a linker capable of removing specific unwanted proteins. In some embodiments, a PROTAC comprises a first domain that interacts with (e.g., specifically binds to) a mitofusin (e.g., Mfn1, Mfn2, etc.) and a second domain that interacts with (e.g., specifically binds to) and E3 ubiquitin ligase. Design and production of PROTACs is generally known, for example as described by Scheepstra et al. (2019) Comput Struct Biotechnol J.

### Biomarkers

Aspects of the disclosure relate to methods for identifying a subject as having a disease associated with mitochondrial dysfunction (e.g., a VPS13D-associated disease). The disclosure is based, in part, on the recognition that increased (e.g., elevated) levels of certain mitofusins (e.g., Mfn2) in a subject are indicative of the subject having one or more mutations in vps13D and/or the subject having a disease associated with mitochondrial dysfunction. In some embodiments, the methods comprise detecting in a biological sample obtained from a subject an increased level of mitofusin (Mfn) expression or activity relative to a control sample.

A “control sample” refers to a sample obtained from a healthy donor (e.g., a subject not having a disease associated with mitochondrial dysfunction, a subject not characterized as having one or more mutations in vps13D, etc.). As used herein, “increased” or “elevated” refers to a level of one or more mitofusins (e.g., Mfn1, Mfn2, etc.) present in a biological sample (e.g., a serum sample) is above a control level, such as a pre-determined threshold or a level of one or more mitofusins in a control sample. Controls and control levels include mitofusin protein levels obtained (e.g., detected) from a subject that does not have or is not suspected of having a disease associated with mitochondrial dysfunction. In some embodiments, a control or control level includes mitofusin protein levels prior to administration of a therapeutic agent (e.g., a modulator of Mfn). An elevated level includes a level that is, for example, 1%, 5%, 10%, 20%, 30%, 40%, 50%, 60%, 70%, 80%, 90%, 100%, 150%, 200%, 300%, 400%, 500%, or more above a control level. An elevated level also includes increasing a phenomenon from a zero state (e.g., no or undetectable Mfn protein expression or level) to a non-zero state (e.g., some or detectable level of Mfn protein expression or presence). In some embodiments, an increase (e.g., increase in the level of one or more Mfn protein levels in the sample relative to a control or a prior sample) can be indicative of a lack of therapeutic efficacy of a therapeutic agent (e.g., therapeutic efficacy in the subject from which the sample was obtained).

Generally, a biological sample can be blood, serum (e.g., plasma from which the clotting proteins have been removed), or cerebrospinal fluid (CSF). However, the skilled artisan will recognize other suitable biological samples, such as certain tissue (e.g., bone marrow, brain tissue, spinal tissue, etc.) and cells (e.g., leukocytes, stem cells, brain cells, neuronal cells, skin cells, etc.). In some embodiments, a biological sample is a blood sample or a tissue sample. In some embodiments, a blood sample is a sample of whole blood, a plasma sample, or a serum sample. In some embodiments, a tissue sample comprises CNS tissue (e.g., brain tissue, spinal tissue, etc.). In some embodiments, a biological sample comprises mitochondria and/or mitochondrial DNA (mtDNA).

In some embodiments, a level of Mfn nucleic acid expression is detected. In some embodiments, the detection comprises performing a gene expression assay on a biological sample. A “gene expression assay” refers to a molecular, biological, or chemical assay which quantifies the relative expression level of a particular gene relative to other genes. In some embodiments, a gene expression assay quantifies the relative expression level of a particular set of genes relative to either 1) other genes or 2) each other gene in the set. Expression levels of genes may be determined by quantifying a level of DNA, RNA (e.g., total RNA, mRNA, miRNA, etc.), or proteins translated as a result of expression of the gene or set of genes.

In some embodiments, a level of Mfn protein expression (translation) is detected. In some embodiments, the detection comprises performing a Western blot. Western blots generally employ the use of a detection agent or probe to identify the presence of a protein or peptide. In some embodiments, detection of one or more Mfn proteins is performed by immunoblot (e.g., dot blot, 2-D gel electrophoresis, etc.), immunohistochemistry (IHC), or ELISA. In some embodiments, the detection agent is an antibody. In some embodiments, the antibody is an anti-Mfn antibody, for example D2D10, D1E9, 6A8, etc.

### EXAMPLES

### Example 1

The mechanisms underlying mitophagy in animals have been defined through studies of derived cell lines. Although these elegant studies of PINK1- and Parkin-dependent mitophagy have significantly advanced our understanding of this important process, studies in animals indicate that the clearance of mitochondria in cells and tissues under physiological conditions do not always utilize the same regulatory pathways. During Drosophila development, the larval intestine undergoes an autophagy driven remodeling process where cells reduce in size and mitochondria are cleared by mitophagy. This system allowed identification of vps13d and other genes as regulators of autophagy under physiological conditions. Importantly, vps13d is an essential and conserved gene that regulates mitochondrial clearance, mitochondrial morphology, and has been implicated in human movement disorders.

Vacuolar protein sorting 13 (vps13) was discovered in yeast, and animals possess four evolutionarily conserved Vps13 family members Vps13A-D. Yeast VPS13, as well as mammalian Vps13A and C, have been implicated in the regulation of inter-organelle contact and lipid transport. However, these studies fail to address whether these human paralogs are repressors or enhancers of membrane contacts. Furthermore, no study has linked VPS13D specifically to regulation of membrane contacts.

Members of the Vps13 family possess unique functional requirements. In contrast to VPS13A-C, VPS13D is one of the most essential genes in human cell lines, and is essential for Drosophila development. Vps13D is the only Vps13 family member that contains a ubiquitin binding domain, which is required for proper mitochondrial morphology and clearance. Vps13D is also the only Vps13 family member in flies that is required for autophagy. Significantly, mutations in VPS13D have been associated with multiple diseases, including a unique group of familial neurological movement disorders involving ataxia, chorea and dystonia.

This example describes vps13d and genes that regulate autophagy and mitochondrial morphology. It was observed that Vps13D acts downstream of Vmp1/EPG-3, a regulator of autophagy and mitochondria and endoplasmic reticulum (ER) contact. Like Vps13D, loss of Vmp1 disrupts autophagy and mitochondrial morphology. Through this relationship, a novel role was identified for Vps13D, as a regulator of mitochondria and ER contact in Drosophila and human cell lines, including fibroblasts derived from patients symptomatic for VPS13D associated neurodegenerative disease. Importantly, it was also observed that Vps13D physically interacts with the regulator of mitochondrial fusion Marf, and that loss of marf suppresses vps13d mutant phenotypes. Data indicate that Vmp1, Vps13D and Marf are important factors in a pathway that regulates inter-organelle contacts in autophagy and mitochondrial morphology.

### Materials and Methods

### Human Cell Lines

All cells were cultured at 37° C. in 5% CO₂ in DMEM supplemented with 5% FBS and Penicillin/Streptomycin.

### vmp1(Δ) and gfp-vmp1Fly Construction

vmp1 loss-of-function, vmp1(Δ), and N terminal GFP-tagged (gfp-vmp1) vmp1 strains were edited using CRISPR/Cas9. For vmp1(Δ), the following sgRNA targeting sequences were used (5′ to 3′): sgRNA1: TGTTGTTGTGACGATTGCTC (SEQ ID NO: 13), sgRNA2: TTACGGGACTAGAAAATCAG (SEQ ID NO: 14). A 200 bp ultramer donor with 100 bp regions flanking the site of the deletion was used to facilitate the deletion, resulting in a single female fly with the deletion that was validated by DNA sequence. For gfp-vmp1, the following sgRNA targeting sequences were used (5′ to 3′): sgRNA3: TGCTGTGACATTTAAGCGGT (SEQ ID NO: 15), sgRNA4: CGAATGCTGTGACATTTAAG (SEQ ID NO: 16). A 2 kb gblock with 1 kb regions flanking the site of insertion and the GFP open reading frame was used to tag the N terminal of vmpl with gfp. A single female fly containing the insertion was collected, and validated by DNA sequencing. For vps13d-3xflag, the following sgRNA targeting sequence was used (5′ to 3′): sgRNA5:TTTATAAAATGCAATAGGT (SEQ ID NO: 17). A 2 kb region flanking the C terminal of genomic vps13d was amplified by PCR and site-directed mutagenesis was used to insert the 3xflag sequence in frame immediately before the stop codon. This fragment was inserted into a TOPO vector via TOPO cloning and sequenced to ensure no additional mutations were present and was used to tag the C terminal of vps13d with 3xflag. A single female fly containing the insertion was collected and validated by DNA sequencing.

### Induction of Mosaic RNAi and Mutant Cell Clones

Mosaic GFP positive RNAi-expressing cell clones were induced. To induce mosaic vmp1(Δ) and vps13d(MiMic) loss-of-function clones, hsflp, FRT19A, mRFP and hsflp;;FRT2A, Ubi-nlsGFP flies were used and crossed with vmp1(Δ) FRT19A/FM7i-pAct-GFP and vps13d(MiMic) FRT2A/TM6B flies, respectively. 8-hour eggs lays were heat shocked for 90 minutes at 37° C.

### Genetic Interaction Analysis

For fly genetic interaction experiments, 20 female flies were mated with 10 male flies for 3 days before being allowed to lay eggs for 48 hours on standard cornmeal agar food and then transferred to new vials. Crosses were allowed to develop until the eclosion of adults, and adult genotypes were quantified based on the presence and absence of dominant genetic markers that are associated with balancer chromosomes.

For worm genetic interaction experiments, N2 Bristol (wild-type) and epg-3(bp933) were used. Worms were cultured at 20° C. on King Agar plates with OP50 E. coli. C25H3.11/vps-13d RNAi bacterial clones and the control bacteria HT115 (expressing empty vector L4440). RNAi clones were confirmed by DNA sequencing. For synthetic lethality assay, five synchronized L1 animals were individually plated on control RNAi plates (L4440). Fifteen synchronized L1 animals were individually plated onto vps-13d RNAi plates. The number of plates exhibiting sterility or larval arrest was then calculated.

### Dissection and Immune-Labeling of Drosophila Larval Intestines

White prepupae were collected and allowed to develop on wet filter paper for 2 hours prior to dissection. Intestines were immuno-stained as previously described with modifications. Intestines were removed in cold PBS before being placed in 4% paraformaldehyde solution for fixation at 4° C. overnight. Intestines were washed twice with PBS and then twice with 0.1% PBSTx before blocking in 5% normal goat serum for 90 minutes and incubation with primary antibody in 0.1% PBSTx overnight. Intestines were then stained with secondary antibody for 3 hours before nuclei staining and mounting. The following primary antibodies were used: rabbit anti-ref(2)p (1:1000), mouse anti-ATP synthase complex V (1:1000, Abcam #ab14748), anti-GFP (1:1000, Abcam #ab13970), rabbit anti-SERCA (1:1000) and anti-VPS13D (1:50). The following secondary antibodies were used: anti-mouse AlexaFluor 647 (Invitrogen #A-21235), anti-rabbit Alexafluor 546 (Invitrogen #A-11035) and anti-chicken AlexaFluor 488 (#A-11039). Nuclei were stained with Hoescht (Invitrogen) and samples were mounted with Vectashield (Vector Lab). Intestines expressing mCherryAtg8a puncta were fixed overnight at 4° C. in 4% paraformaldehyde before being imaged the next day. Images were acquired using a Zeiss LSM 700 confocal microscope.

### Transmission Electron Microscopy

Transmission electron microscopy (TEM) was conducted. Intestines were dissected in PBS (GIBCO) 2 hours after pupation and fixed in a solution of 2.5% glutaraldehyde and 2% paraformaldehyde in 0.1M sodium cacodylate buffer, pH 7.4 (Electron Microscopy Sciences) for 1 hour at room temperature followed by overnight fixation at 4° C. in fresh fix. Intestines were washed in 0.1M sodium cacodylate buffer, pH 7.4, post-fixed in 1% osmium tetroxide in distilled water for 1 hour at room temperature and washed in distilled water. Preparations were stained en bloc in 1% aqueous uranyl acetate for 1 hour at 4° C. in the dark, washed in distilled water, dehydrated through a graded ethanol series, treated with propylene oxide and infiltrated in SPI-pon/Araldite for embedding. Ultrathin sections were cut on a Leica UC7 microtome. Sections were stained with uranyl acetate and lead citrate and examined on a Phillips CM10 TEM. Images were taken down the length of the anterior region of the midgut to ensure an unbiased approach. For each genotype, at least 3 intestines were embedded and sectioned for analyses and quantification. All images were reviewed and representative images selected for analyses.

For cell culture, plated cells were prefixed in 50% media: 50% fix, 2.5% glutaraldehyde and 2% paraformaldehyde in 0.1M sodium cacodylate buffer, pH 7.4 (Electron Microscopy Sciences) for 5 minutes followed by fixation in full fix for 1 hour at room temperature. Cells were then washed with 0.1M cacodylate buffer, pH 7.4, post-fixed in 1% osmium tetroxide in distilled water for 1 hour at room temperature and washed in distilled water. Preparations were stained en bloc in 1% aqueous uranyl acetate over night at 4° C. in the dark and then washed in distilled water. The cells were then scraped and pelleted. Cell pellets were embedded in agarose, dehydrated through a graded ethanol series, treated with propylene oxide and infiltrated in SPI-pon/Araldite for embedding. Ultrathin sections were cut on a Leica UC7 microtome. Sections were stained with uranyl acetate and lead citrate and examined on a Phillips CM10 TEM. For each cell line, at least (3) 10 cm2 dishes at 60-80% confluency were embedded independently of each other and sectioned in an unbiased manner for analyses and quantification.

### Western Blot and Immunoprecipitation

Tissue was lysed in 1× Laemli Sample Buffer diluted in RIPA lysis buffer (10 mM Tris-Cl PH 8.0, 1 mM EDTA PH 8.0, 0.5 mM EGTA, 2.4 mM Sodium Deoxycholate 140 mM Sodium Chloride) at a ratio of 10 μL lysis buffer per intestine and 30 μL per whole pupa. Samples were crushed in solution using a plastic pestle for 30 seconds before being boiled at 99° C. for 6 minutes. Samples were run on 7.5% polyacrylamide gel, transferred onto 0.45 μm PVDF membranes (Millipore Sigma), and probed with antibodies using standard protocols. Primary antibodies used were mouse anti-FLAG (1:1000, Millipore Sigma), rabbit anti-Marf (1:1000, from Alexander Whitworth), mouse anti-Actin (1:1000, Proteintech), and mouse anti-ATP synthase complex V (1:1000, Abcam).

For immunoprecipitations, 2-hour-old pupae were lysed in RIPA lysis buffer supplemented with 1 mM NEM, 1 mM PMSF and Halt Protease Inhibitor Cocktail (Thermo Fisher) at a ratio of 16 pupae per 250 μL lysis buffer. Pupae were crushed with a plastic pestle for 30 seconds and incubated on ice for 30 minutes before being centrifuged at 4° C. at 13,000 rpm for 10 minutes. Supernatant was filtered through 0.45 μm Cellulose Acetate filters (Millipore Sigma). 30 μL of filtered supernatant was diluted in 10 μL of 4× Laemli Sample Buffer (Biorad), boiled for 6 minutes at 99° C. and used as input. 200 μL of filtered supernatant (approximately 1 mg protein) was used for immunoprecipitation. 40 μL of anti-FLAG M2 magnetic bead slurry (Millipore Sigma) warmed to room temperature was washed twice with RIPA buffer before incubation with filtered supernatant for 2 hours at 4° C. on a rotator. Following incubation, supernatant was discarded, and beads were washed 4 times with 1 mL 0.1% PBST. Beads were eluted with 20 μL× Laemli Sample Buffer diluted in RIPA lysis buffer and boiled for 6 minutes at 99° C. 20 μL of input and eluate was run on 7.5% polyacrylamide gel for Western Blot analysis.

### Vmp1 Regulates Autophagy, Mitophagy, and Mitochondrial Morphology

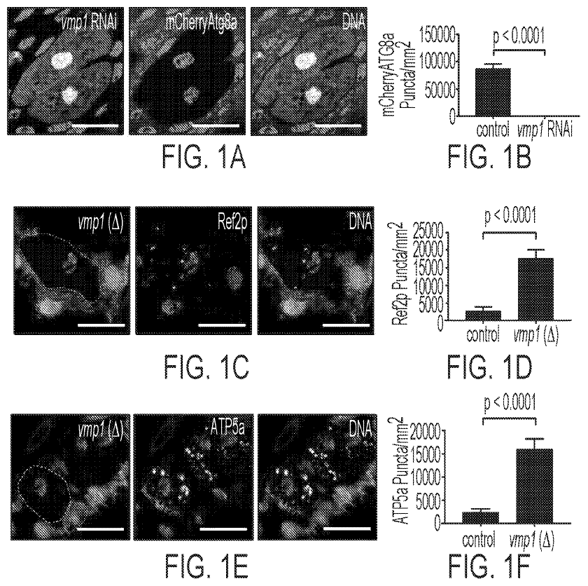

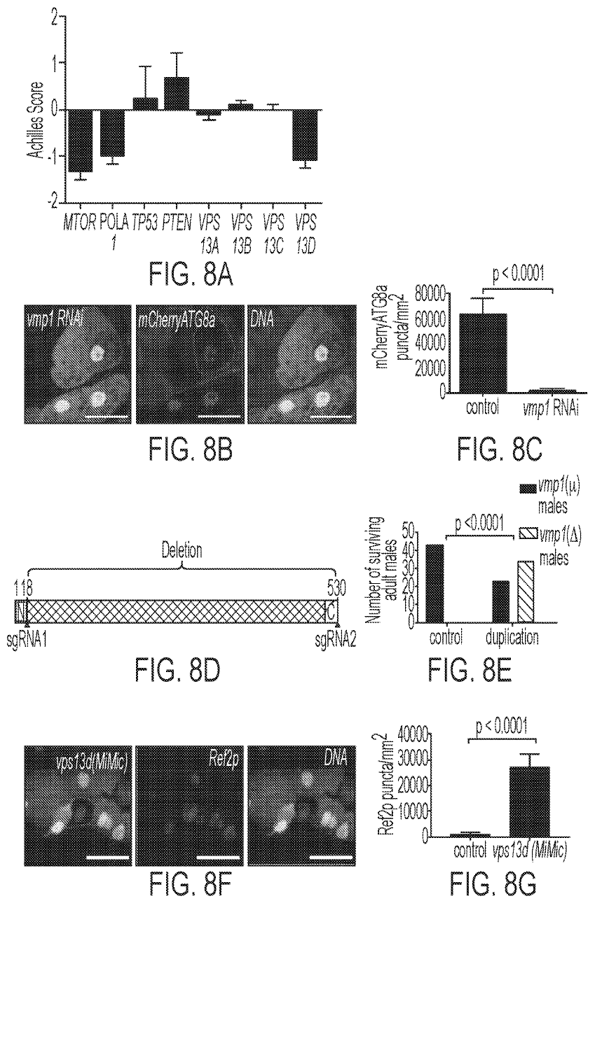

The essentiality (FIGS. 8A-8G) and unique role of Vps13D in autophagy among Vps13 family members prompted consideration of whether other factors implicated in both autophagy and inter-organelle contact may possess phenotypes that are similar to vps13d. Vmp1 (EPG-3 in C. elegans) is a conserved regulator of autophagy in worms and mammals, and also influences inter-organelle contacts. To test if Drosophila Vmp1 (also known as Tango5) has a similar function to Vps13D, the function of vmp1 was analyzed in larval intestine cells where vps13d functions in autophagy, cell size reduction, mitochondrial clearance and maintenance of mitochondrial size. Like vps13d mutant cells, cells with reduced Vmp1 function that express vmp1 RNAi and green fluorescent protein (GFP) did not accumulate mCherryAtg8a autophagy reporter puncta and were unable to reduce in size, unlike neighboring GFP-negative control cells (FIGS. 1A-1B). This was the same phenotype seen in vps13d RNAi-expressing and loss of function intestine cells. Similar results were obtained using a distinct RNAi targeting a different vmp1 sequence (FIGS. 8B-8C).

CRISPR/CAS9 gene editing was used to create a loss-of-function vmp1 mutant Drosophila named vmp1(Δ) (FIG. 8D). Homozygous vmp1(Δ) mutant animals die during development with a small number of animals surviving until the 3rd instar larval stage. Importantly, an X chromosome duplication containing the vmpl open reading frame complemented the vmp1(Δ) lethal phenotype (FIG. 8E).

Next, vmp1(Δ) mutant cells were analyzed for phenotypes that are similar to homozygous vps13d mutant intestine cells. Consistent with vmp1 RNAi knockdown, intestines with homozygous vmp1(Δ) mutant cells lacking red fluorescent protein (RFP) accumulated the autophagic cargo receptor Ref2p (p62 in mammals) compared to neighboring control cells that possess RFP (FIGS. 1C-1D), indicating that autophagy is impaired. Similar to homozygous vmp1(Δ) mutant cells, Ref2p accumulated in vps13d (MiMic) mutant cells (FIGS. 8F-8G).

Mitochondria are cleared by autophagy during intestine development. Therefore, whether Vmp1, like Vps13D, is required for clearance of mitochondria in the intestine was investigated. Significantly, homozygous vmp1(Δ) mutant intestine cells lacking RFP were unable to clear mitochondria compared to neighboring control cells that express RFP based on persistence of the mitochondrial protein ATP5a (FIGS. 1E-1F). Combined, these data indicate that Vmp1 has similar functions to Vps13D, including the regulation of autophagy and clearance of mitochondria.

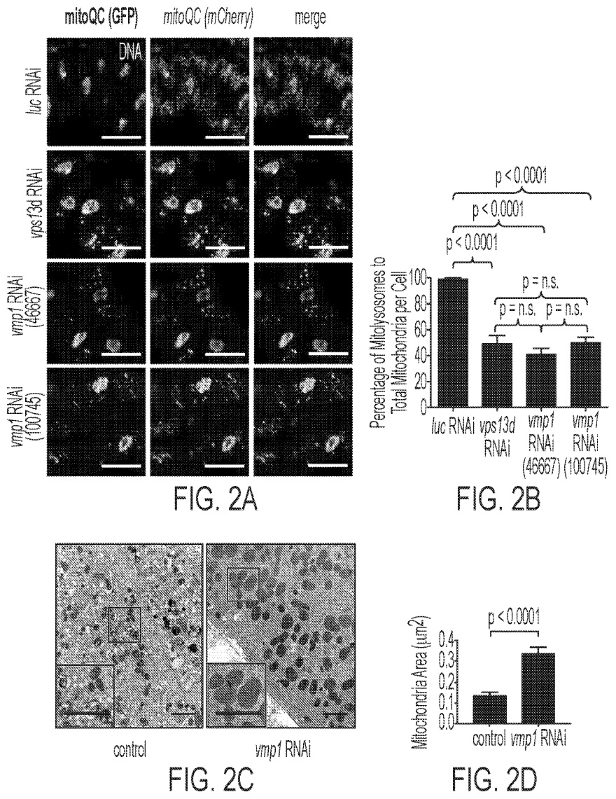

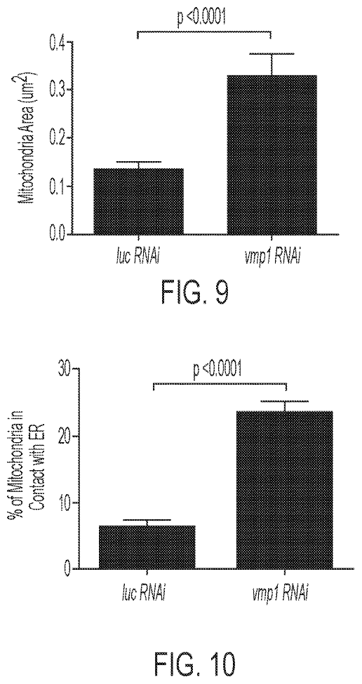

Nest, whether the presence of mitochondria in homozygous vmp1(Δ) mutant intestine cells was due to a defect in mitophagy was investigated. The mito-QC system, which utilizes a mitochondrial protein tagged with GFP and RFP to detect when mitochondria are delivered to autolysosomes, was used. Control intestines that expressed control luc RNAi cleared most mitochondria by 2 hours after pupation as shown by the presence of RFP-positive and GFP-negative puncta (FIGS. 2A-2B). By contrast, intestines that expressed vps13d RNAi or expressed either of two distinct vmp1 RNAi constructs retained mitochondria that were both RFP- and GFP-positive 2 hours after pupation (FIGS. 2A-2B). In addition, transmission electron microscopy (TEM) analyses revealed enlarged mitochondria in vmp1 RNAi-expressing intestine cells compared to control intestine cells at 2 hours after pupation (FIGS. 2C-2D). Larger mitochondria were also observed by TEM analyses of intestine cells expressing a different vmpl RNAi (FIG. 9). These data indicate that Vmp1 and Vps13D have similar functions in regulating autophagy, mitophagy and mitochondrial morphology in Drosophila intestines.

### Vps13D Regulates Mitochondria and Endoplasmic Reticulum Contact

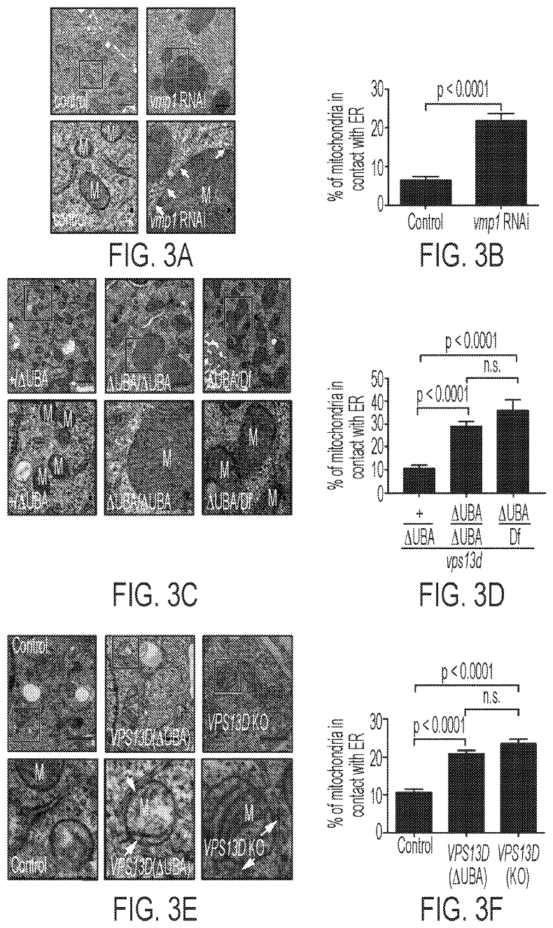

Vmp1 is a repressor of membrane contact, and the failure to disassemble mitochondria and endoplasmic reticulum (ER) contact alters mitochondrial morphology in vmp1 mutant mammalian and C. elegans cells. Whether Vmp1 influences mitochondria and ER contact in Drosophila through TEM analyses of intestines 2 hours after pupation was investigated. Intestines with decreased Vmp1 function that express vmp1 RNAi possessed increased contact between mitochondria and ER compared to luciferase (luc) RNAi control cells (FIGS. 3A-3B). Similar results were obtained with a different vmp1 RNAi line (FIG. 10). These data indicate that Vmp1 regulates mitochondria and ER contact in Drosophila.

Given the role of Vmp1 in mitochondria and ER contact, as well as the similarities between vmp1 and vps13d mutant cell phenotypes, whether or not vps13d functions in mitochondria and ER contact was investigated by TEM analyses. Intestine cells of either homozygous vps13d (ΔUBA), a mutant lacking the ubiquitin binding domain, or vps13d(ΔUBA)/chromosome deficiency (Df) for the vps13d genomic region had significantly increased mitochondria and ER contact compared to heterozygous vps13d (ΔUBA)/wild type control cells 2 hours after pupation (FIGS. 3C-3D).

HeLa cells that either lack the ubiquitin binding domain, VPS13D(ΔUBA), or are thought to be a strong loss-of-function mutant, VPS13D(KO) were analyzed. Significantly, it was observed that mitochondria and ER contact were increased in both VPS13D mutant human HeLa cell lines (FIGS. 3E-3F). These data indicate that both vmp1 and vps13d regulate mitochondria and ER contact in Drosophila and human cells.

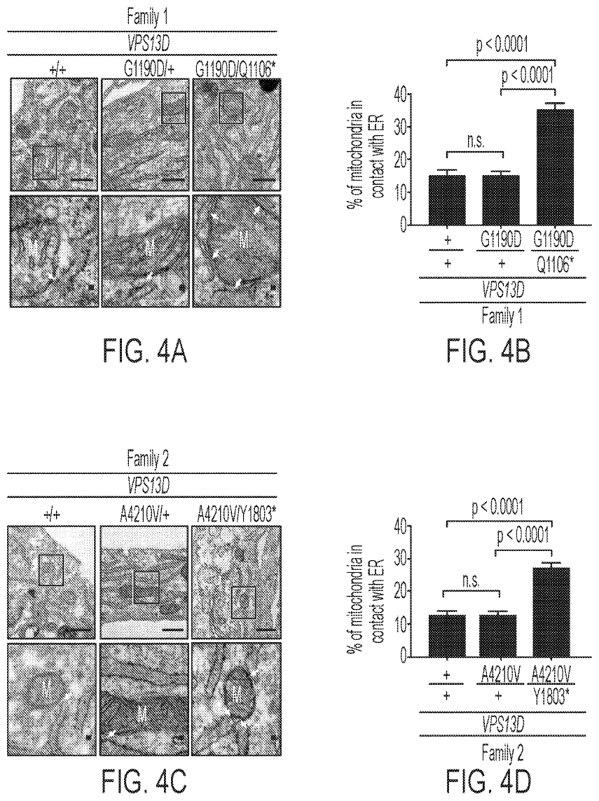

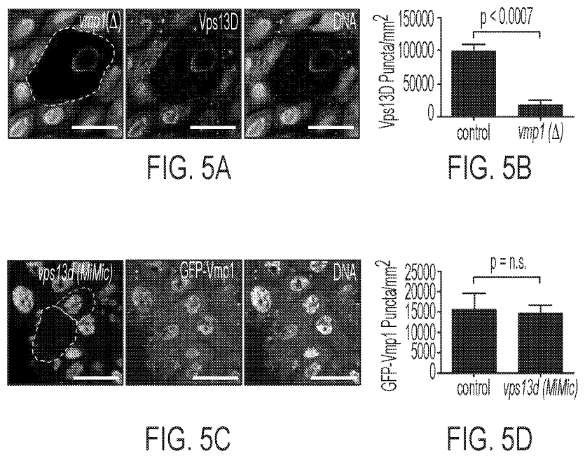

Mutations in VPS13D have been associated with familial neurological movement disorders, including ataxia, dystonia, and chorea. Given the conserved function of VPS13D in inter-organelle contact between fly and human HeLa cells, patient-derived cells with VPS13D mutations were investigated by TEM to detect whether altered mitochondria and ER contact. Remarkably, mitochondria in fibroblasts that were derived from the symptomatic VPS13D mutant (G1190D/Q1106*) patient had increased mitochondria and ER contact compared to the mitochondria in fibroblasts derived from a relative (G1190D/+) and unrelated control (FIGS. 4A-4B). In addition, mitochondria and ER contact was analyzed in a second set of fibroblasts derived from an unrelated family with symptoms associated with the VPS13D mutations. Mitochondria from the symptomatic VPS13D mutant patient from this family (A4210V/Y1803*) also exhibited increased mitochondria and ER contact compared to mitochondria in fibroblasts derived from both an asymptomatic relative (A4210V/+) and a separate unrelated control (FIGS. 4C-4D). Therefore, VPS13D regulates mitochondria and ER contact, this function is conserved from flies to humans, and this phenotype likely contributes to cell health and neurological disease.

### Vps13D Functions Downstream of Vmp1 to Regulate Mitochondrial Morphology and Mitophagy

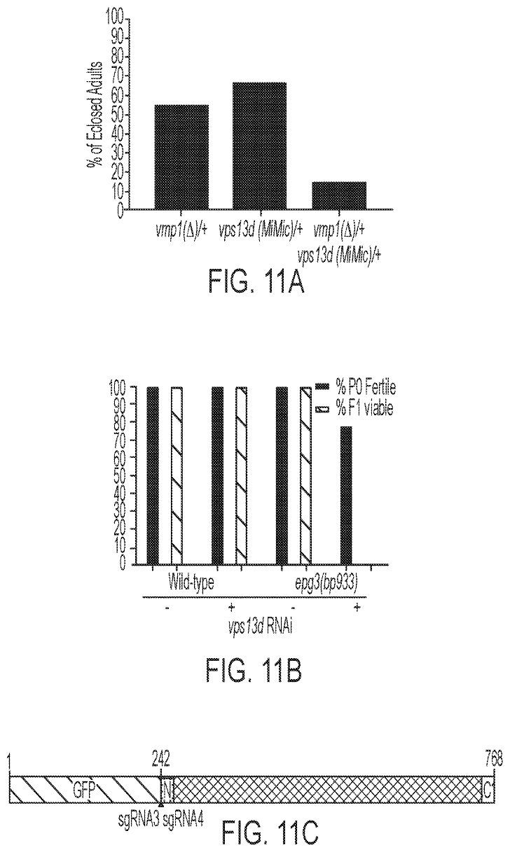

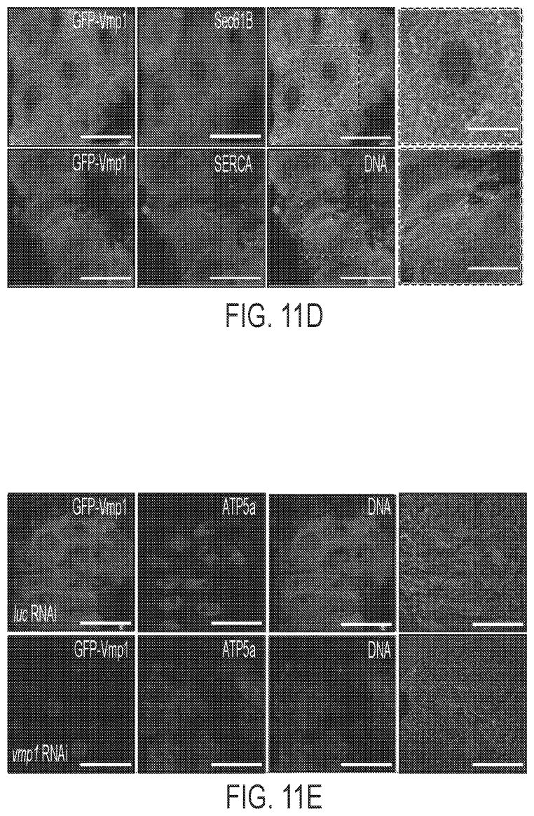

The similarities between vmp1 and vps13d mutant phenotypes indicates that these genes may be in the same genetic pathway. Consistent with being in the same pathway, vmp1 and vps13d genetically interacted in both Drosophila and C. elegans (FIGS. 11A-11B). These data prompted further investigation of the relationship between these factors in Drosophila intestine cells. Vps13D protein localization in control and homozygous vmp1(Δ) mutant intestine cells 2 hours after pupation were analyzed; it was observed that Vps13D protein puncta were significantly decreased in vmp1 mutant cells compared to neighboring control cells (FIGS. 11A-11B). These data indicate that Vps13D puncta are dependent on Vmp1.

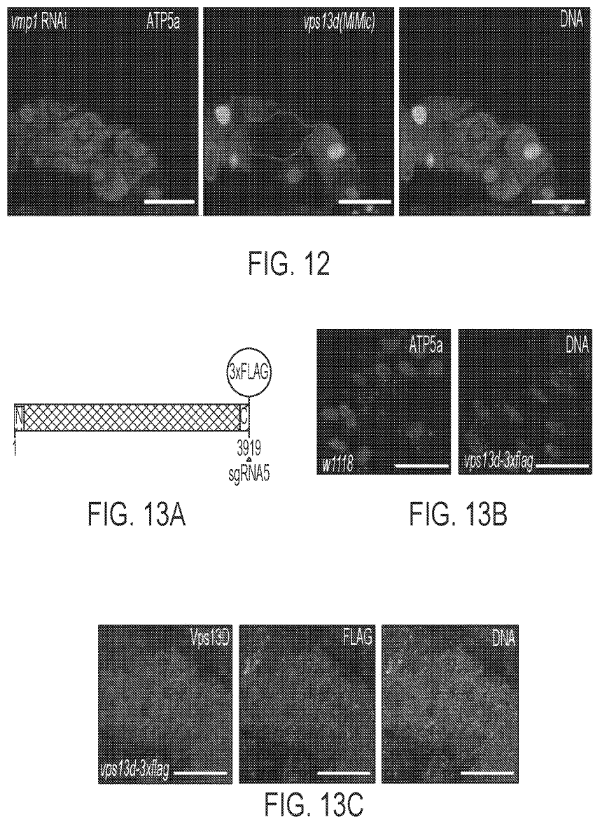

Next, whether Vps13d influences Vmp1 was tested. CRISPR/CAS9 was used to tag Vmp1 with GFP on the N terminus (GFP-Vmp1) (FIG. 11C). These flies are viable, fertile and complemented the lethal phenotype associated with the vmp1(Δ) mutant. GFP-Vmp1 co-localizes with the ER markers Sec61β and Serca in intestine cells (FIG. 11D). In addition, vmp1 RNAi expression in GFP-Vmp1 larval intestines decreased GFP-Vmp1 puncta, and resulted in retention of mitochondria and increased cell size compared to controls (FIG. 11E). Interestingly, vps13d(MiMic) loss-of-function mutant cells did not possess altered GFP-Vmp1 localization (FIGS. 12C-12D), indicating that Vmp1 localization is not dependent on vps13d function. Combined, these data indicate that Vps13d functions downstream of Vmp1.

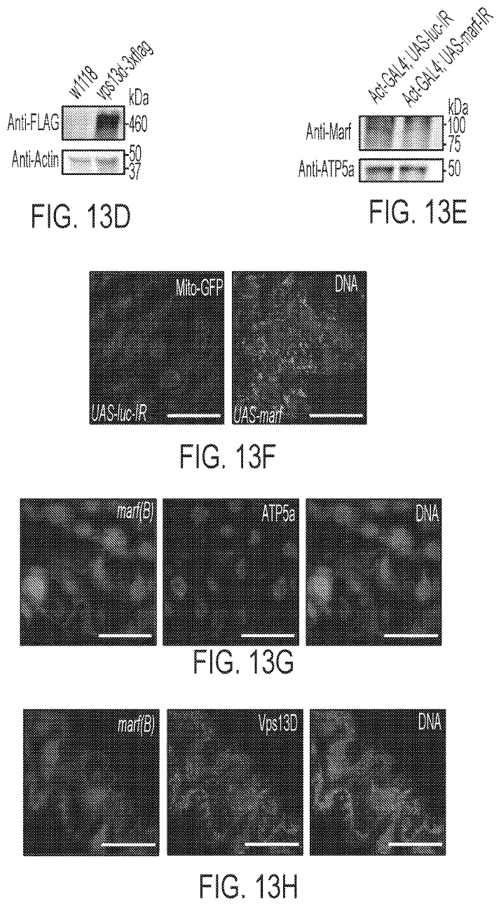

The relationship of Vmp1 and Vps13d in the clearance of mitochondria was investigated. Mitochondrial clearance in vmp1(Δ) and vps13d(ΔUBA) double mutant intestine cells with vmp1(Δ)/+ and vps13d(ΔUBA) single mutant control cells 2 hours after pupation was investigated. Double mutant cells had similar amounts of mitochondrial ATP5a protein compared to neighboring control cells (FIGS. 13A-13B), indicating that these genes function in the same pathway to clear mitochondria. Consistent with these findings, loss-of-vps13d (MiMic) function failed to enhance the mitochondrial clearance phenotype caused by expression of vmp1 RNAi throughout the intestine (FIG. 12).

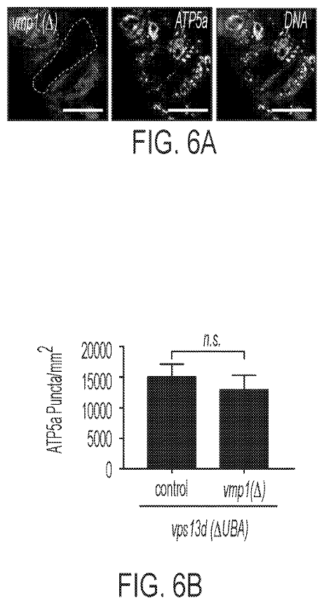

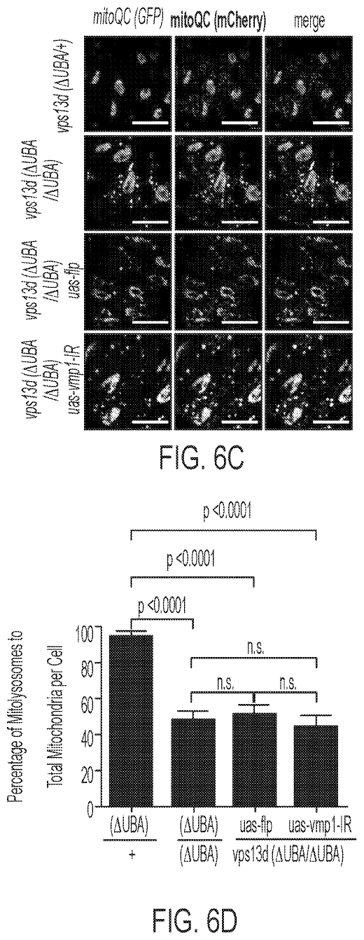

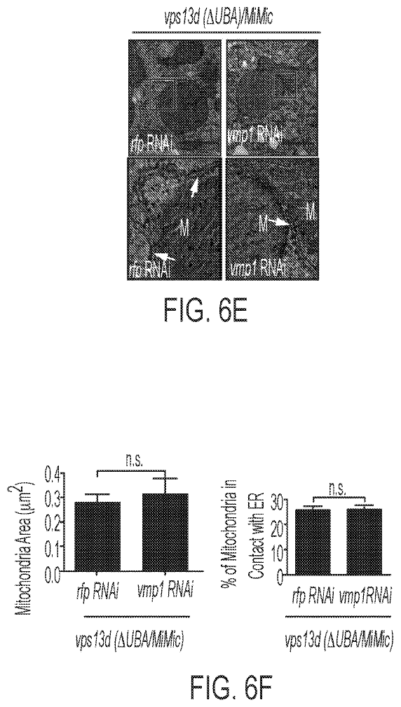

Mito-QC was used to investigate whether Vmp1 and Vps13D function in a shared mitophagy pathway. Control intestines that were heterozygous for the vps13d (ΔUBA) mutation cleared most mitochondria by 2 hours after pupation as shown by the presence of RFP-positive and GFP-negative puncta. By contrast, intestines that were homozygous for the vps13d (ΔUBA) mutation retained mitochondria that were both RFP- and GFP-positive 2 hours after pupation (FIGS. 6C-6D). Combined knockdown of vmp1 by RNAi in a homozygous vps13d (×UBA) mutant background failed to enhance the vps13d mutant mito-QC phenotype (FIGS. 6C-6D), further indicating that vmp1 and vps13d function in the same mitophagy pathway.

To investigate if Vps13D and Vmp1 function in the same pathway to regulate mitochondria and ER contact, vps13d (ΔUBA)/Df expressing either vmp1 or control rfp RNAi were analyzed by TEM. Importantly, the combined reduction of both vmp1 and vps13d function failed to enhance either the increased mitochondrial size or mitochondria and ER contact phenotypes compared to the loss of vps13d alone (FIGS. 6E-6F). These data indicate that Vps13D and Vmp1 function in the same pathway to regulate mitophagy and mitochondria and ER contact, and that Vps13D functions downstream of Vmp1.

### Vps13D Physically Interacts with Marf and Loss of marf Suppresses vps13D Mutant Phenotypes

CRISPR was used to tag the endogenous Drosophila vps13d gene with 3xflag on the C terminus of the open reading frame (FIG. 13A). Unlike the vps13d mutants, these flies are viable, fertile, and do not have altered mitochondrial morphology in intestine cells at 2 hours after pupation (FIG. 13B). Co-staining of intestine cells with anti-FLAG and anti-Vps13D at 2 hours after pupation revealed colocalization (FIG. 13C). In addition, western blot analyses of pupal lysates revealed the presence of a distinct band of approximately 450 kDa in vps13d-3xflag flies that was absent in the control w1118 flies, the approximate size of Vps13D-3xFLAG (FIG. 13D).

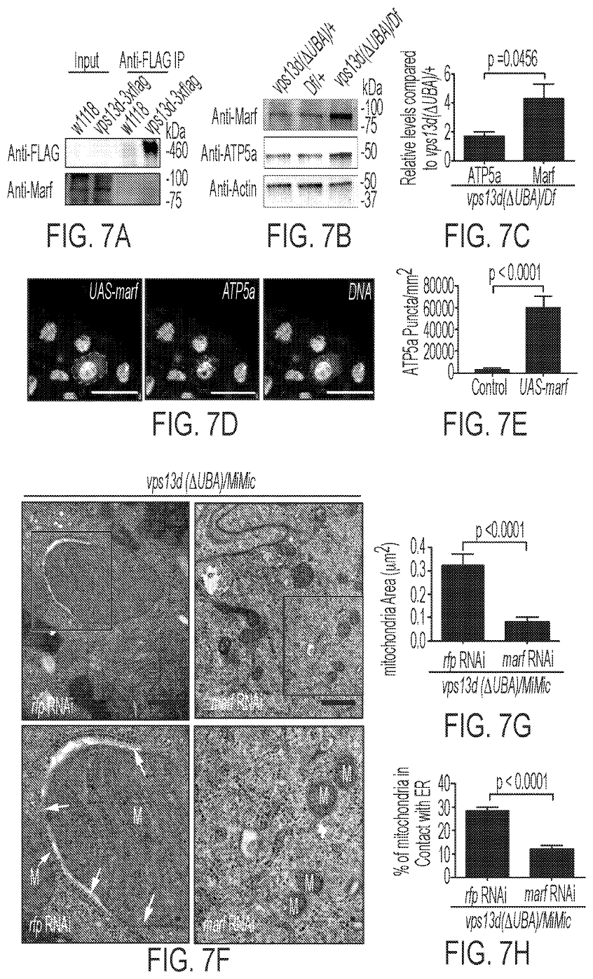

The 3xFLAG epitope was used to immunoprecipitate Vps13D and potential interacting proteins. Probing with a Marf-specific antibody revealed the presence of a specific band in the vps13d-3xflag eluate that was absent in the w1118 negative control eluate (FIGS. 7A and 13E), indicating that Vps13d and Marf physically interact. Furthermore, intestines from vps13d (ΔUBA)/Df trans-heterozygous mutants have increased levels of Marf compared to vps13d(ΔUBA)/+ and Df/+controls 2 hours after pupation (FIG. 7B). This increase in Marf was greater than the increase in ATP5a, indicating that this effect on Marf levels was not simply due to a non-specific failure to clear mitochondria (FIG. 7C). These data indicate that Vps13D influences Marf levels.

The role that Marf may play in mitochondrial clearance in intestines 2 hour after pupation was investigated. Overexpression of Marf inhibited mitochondrial clearance (FIGS. 7D-7E), a phenotype that is similar to vps13d loss of function mutants. Similar results were obtained by expression of marf in all intestine enterocyte cells 2 hours after pupation (FIG. 13F). Unlike vps13d loss-of-function mutants, marf(B) loss-of-function mutant cells did not possess a defect in mitochondrial clearance (FIG. S6G). In addition, marf(B) mutant cells did not have altered Vps13d puncta (FIG. 13H), indicating that Vps13D functions upstream of Marf in the regulation mitochondrial clearance and morphology.

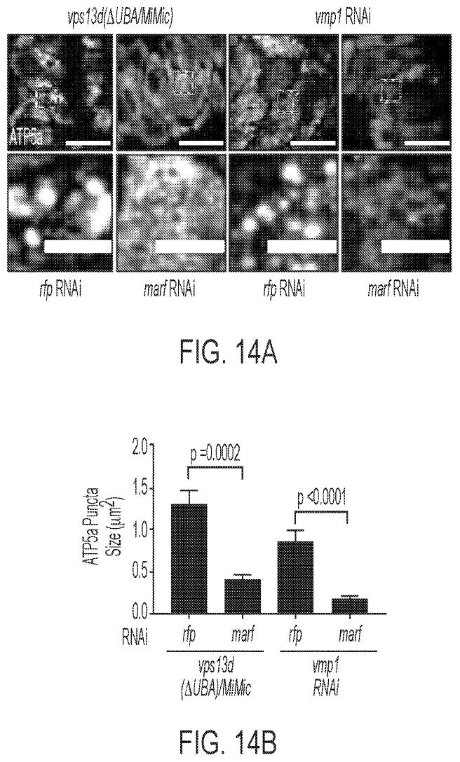

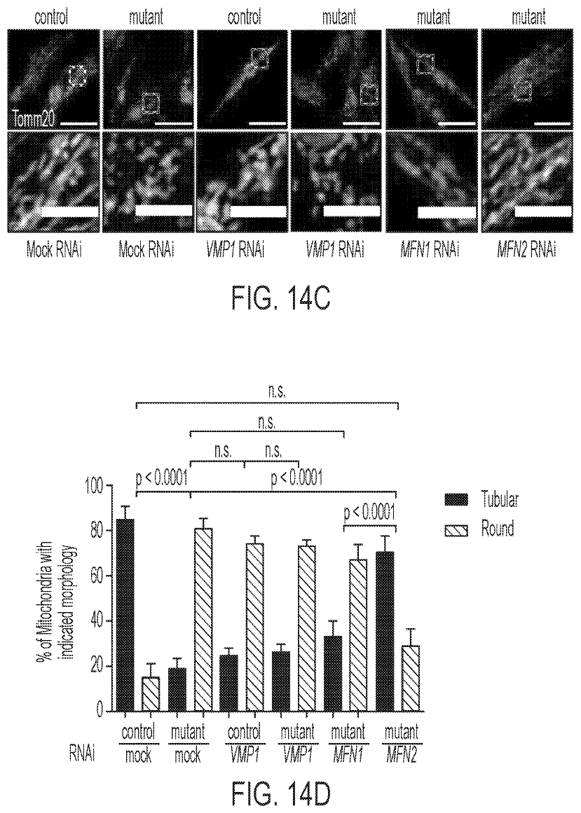

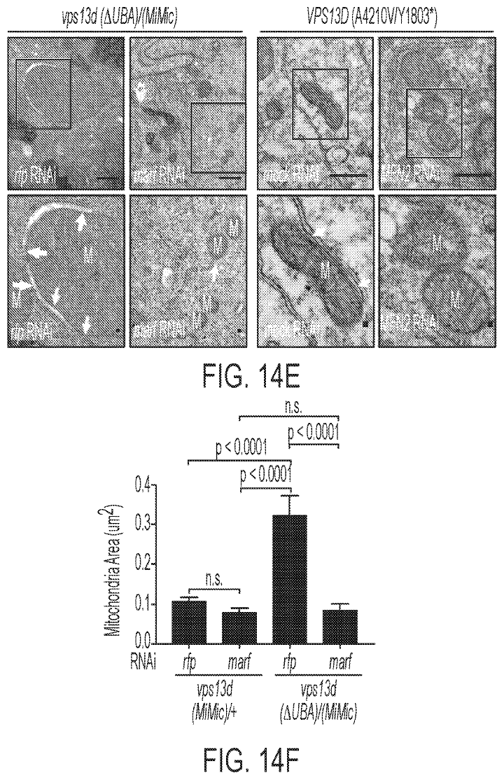

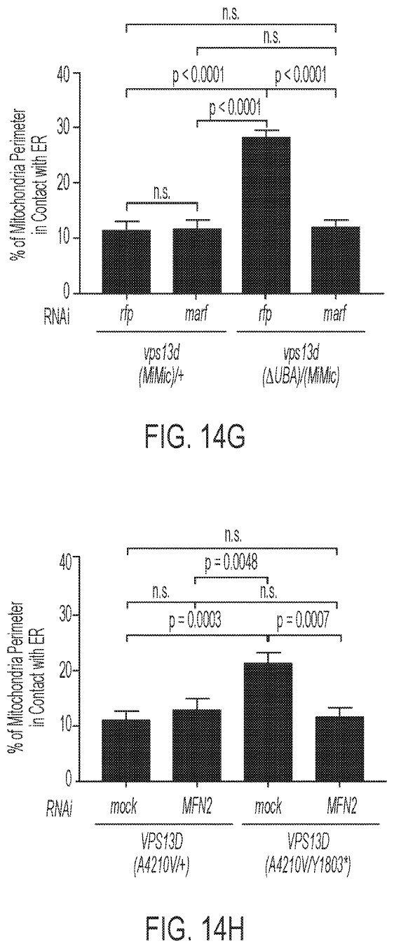

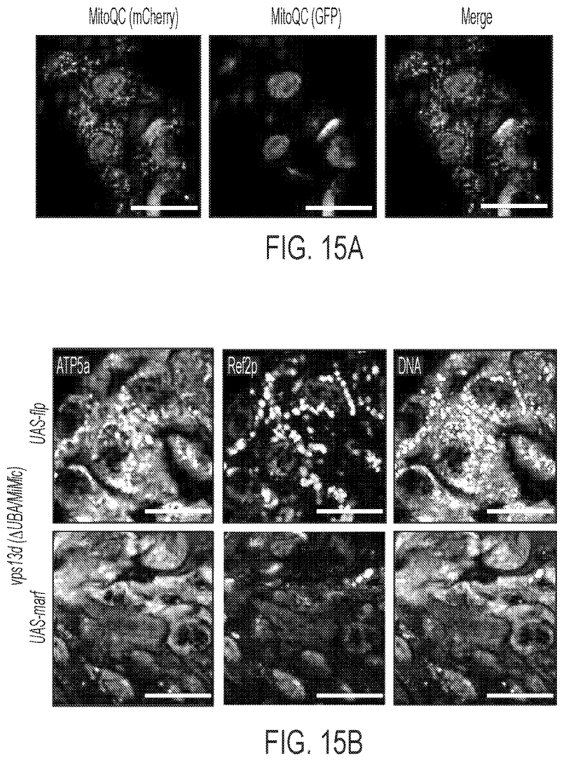

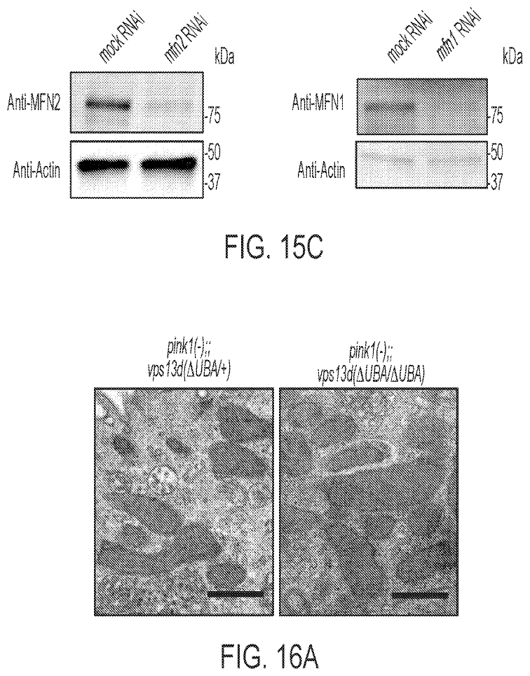

MFN2 is an established mitochondria and ER tether that regulates mitochondrial dynamics and mitophagy. Given the physical and genetic relationship between Vps13d, Vmp1, and Marf, it was investigated whether Vmp1 and Vps13D regulates mitochondria morphology and mitochondria and ER contact sites upstream of Marf. Knockdown of marf suppressed the enlarged mitochondrial phenotypes seen in vps13d (ΔUBA/MiMic) mutants and vmp1 knockdown intestine cells (FIGS. 14A-14B). Knockdown of marf also suppressed the Mito-QC and Ref2p accumulation phenotype in vps13d mutant intestine cells (FIGS. 15A-15B). Consistent with findings in other cell lines, knockdown of VMP1 in heterozygous control fibroblasts increased the number of round mitochondria, similar to the VPS13D mutant patient-derived fibroblasts. VMP1 knockdown in patient-derived fibroblasts did not significantly increase the ratio of round mitochondria to tubular mitochondria, indicating that like in Drosophila intestines, VMP1 and VPS13D are functionally linked in a pathway in human fibroblasts.

Significantly, MFN2 knockdown in patient-derived fibroblasts (FIG. 15C) also suppressed the abnormal mitochondrial phenotype in VPS13D mutant patient-derived fibroblasts (FIGS. 14C-14D). Interestingly, MFN1 knockdown in patient-derived fibroblasts (FIG. 15C) did not suppress this VPS13D associated phenotype (FIGS. 14C-14D). These findings indicate that the mechanistic relationship between VPS13D, VMP1, and Marf/MFN2 are conserved from Drosophila to humans, and that this relationship likely contributes to disease pathology.

Next, whether or not decreased marf/MFN2 function can suppress the vps13d mutant intestine cell mitochondria and ER contact phenotype was investigated. Consistent TEM analyses of mitochondria in vps13d RNAi-expressing intestine cells, reduction of marf function by RNAi suppresses the enlarged mitochondrial phenotype in vps13d (ΔUBA)/MiMic mutants (FIGS. 14E-14F). Significantly, expression of marf RNAi also suppressed the increased mitochondria and ER contact phenotype in vps13d mutant intestine cells (FIG. 14E and FIG. 14G). Importantly, MFN2 knockdown in VPS13D mutant fibroblasts also suppressed the mitochondria and ER contact phenotype (FIG. 14E and FIG. 14H). Therefore, these data indicate that Vps13d mechanistically regulates mitochondria and ER contact sites through Marf/MFN2 in Drosophila and human fibroblasts.

### Example 2

Data described herein indicates that Vps13D physically interacts with Mfn2. It is investigated whether Mfn1 or Mfn2 protein levels are elevated in Vps13D mutant patient-derived fibroblasts compared to control fibroblasts. Knockdown of Mfn2 (or Mfn1) by siRNA is also performed to assess whether it suppresses the Vps13D mutant mitochondria and ER contact phenotype in patient-derived fibroblasts.

Screening for regulators of Marf/Mfns is conducted using the fly system because it has less genetic redundancy. In some embodiments, protein levels of either Marf, Mfns or both are influenced by both E3 ubiquitin ligases and kinases.

### Example 3

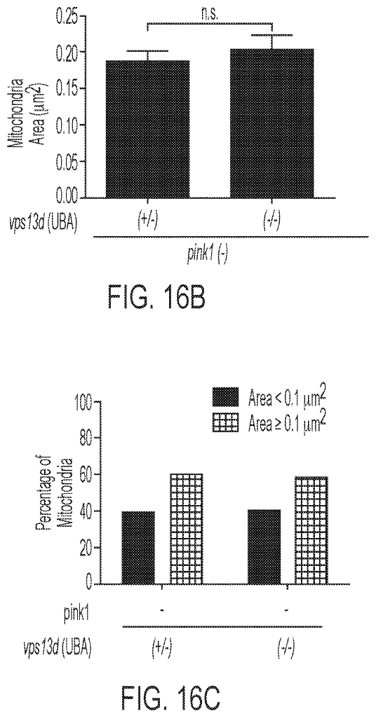

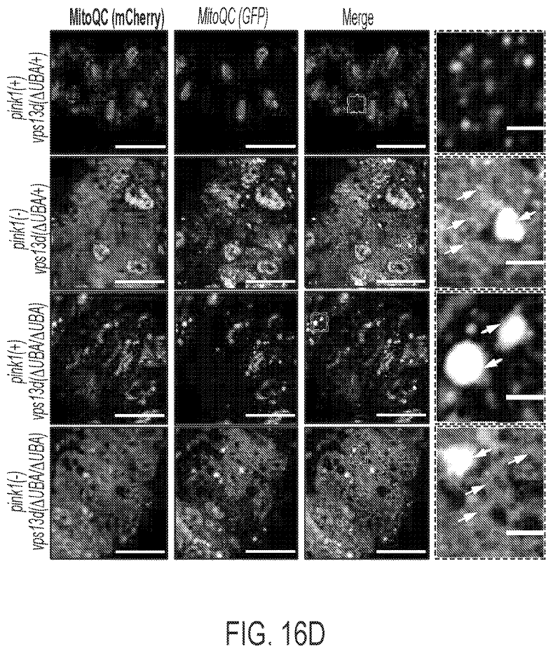

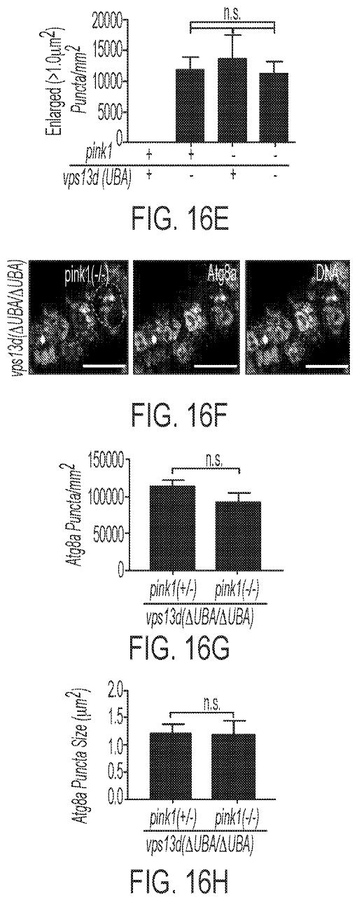

Similarities in Drosophila vps13d and pink1 mutant cell phenotypes were observed. This example describes double-mutant genetic analyses to determine whether vps13d and pink1 are in a common pathway that regulates mitochondria structure and removal. Mitochondrial size was compared in TEM sections of pink1 mutant with pink1;vps13d double-mutant intestine cells. These mutants had similar mitochondria area 2 h after puparium formation (FIGS. 16A-16B). Furthermore, these single- and double-mutant genotypes had a similar proportion of remaining mitochondria that are <0.1 μm2 (FIG. 16C), even though vps13d (ΔUBA) single mutants have larger mitochondria and fewer mitochondria that are <0.1 μm2 than control and pink1^{B9} single-mutant intestines. These data indicate that the combined loss of both vps13d and pink1 fails to enhance single-mutant mitochondrial size phenotype, thus indicating that Vps13D functions in the same pathway as Pink1 in the regulation of mitochondrial size.

Whether Vps13D and Pink1 act within the same pathway to regulate mitophagy was also investigated. The Mito-QC system, which utilizes mitochondria-localized tandem mCherry and GFP fluorescent tags, to label mitochondria outside of autolysosomes with both mCherry and GFP and mitochondria inside autolysosomes with only mCherry as the acidic environment of the autolysosome quenches GFP signal, was used to analyze mitophagy. In control cells that are wild-type for pink1 and heterozygous for vps13d, most of the GFP signal from the Mito-QC was quenched, leaving only mCherry puncta 2 h after prepupa formation (FIGS. 16D-16E) and indicating that mitophagy was active. By contrast, pink1 mutants and pink1;vps13d double-mutant cells retained both GFP and mCherry signal that was absent in the control (FIGS. 16D-16E), indicating that mitophagy was impaired. It is worth noting that the morphology of the retained GFP and mCherry signal differed between these single and double mutants. The pink1 mutants appeared as either filamentous structures or large, round, and punctate structures. By contrast, vps13d mutants only had the enlarged punctate structures (FIG. 16D). Importantly, the distribution of the large and round yellow puncta were the same in pink1 and pink1;vps13d double-mutant cells (FIGS. 16D-16E). Together with TEM data (FIGS. 16A-16C), these findings indicate that Vps13D and Pink1 function in a common pathway to regulate mitochondrial morphology and clearance.

To further investigate the relationship between Vps13D and Pink1, the influence of these genes on Atg8a puncta in intestine cells 2 h after puparium formation was investigated. Like vps13d mutant intestine cells, pink1 mutant intestine cells possess abnormal and enlarged Atg8a localization. Both vps13d mutant (labeled by nuclear RFP) and pink1;vps13d double-mutant (lacking nuclear RFP) intestine cells possessed similar Atg8a puncta size and amounts (FIGS. 16F-16H). These findings indicate that pink1 and vps13d function in the same pathway to regulate Atg8a localization.

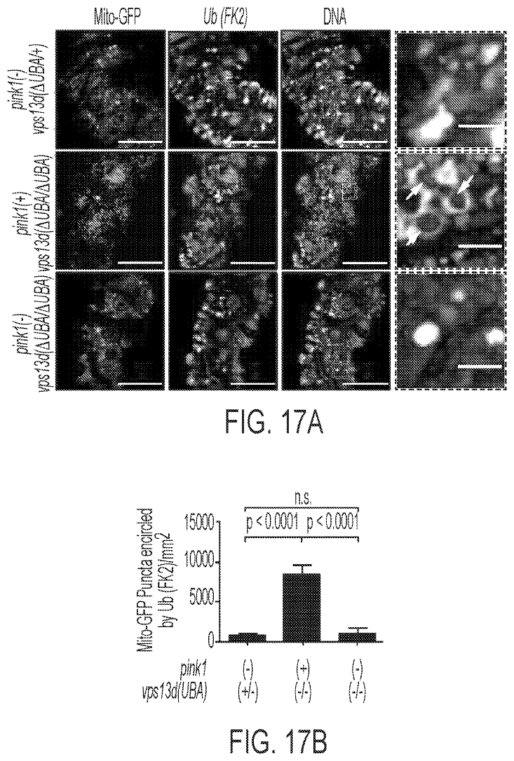

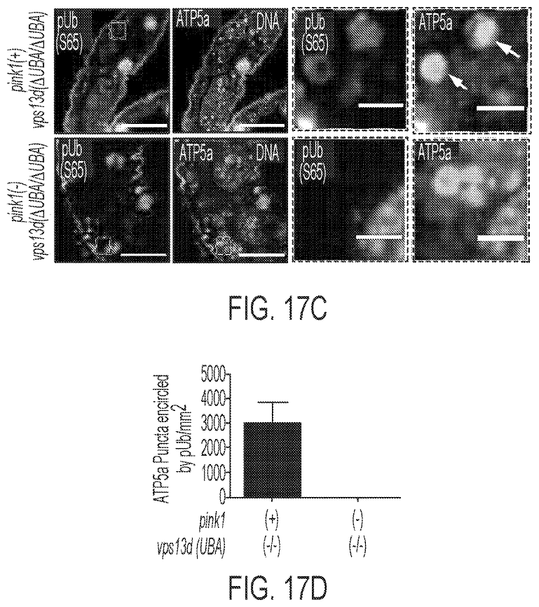

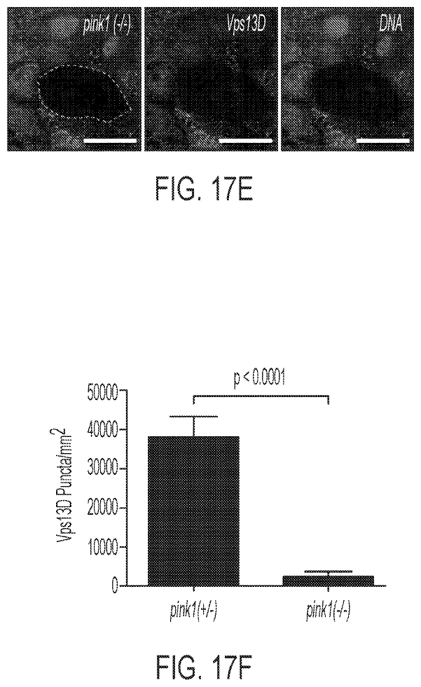

Pink1 senses mitochondrial stress and facilitates ubiquitination of mitochondria-associated proteins to facilitate mitophagy. In contrast to Atg8a localization, pink1 and vps13d mutant intestine cells differ in conjugated ubiquitin localization. To further examine the relationship between these two regulators of mitochondrial clearance, conjugated ubiquitin localization in either pink1 mutant, vps13d mutant, or pink1;vps13d double-mutant cells was investigated. In contrast to vps13d mutant cells, pink1;vps13d double-mutant cells exhibited the same pattern of conjugated ubiquitin localization as pink1 single-mutant cells that was not associated with the perimeter of mitochondria (FIGS. 17A-17B). Furthermore, there was no additive increase in the remaining mitochondria in pink1 single-mutant cells compared with pink1;vps13d double-mutant cells 2 h after pupariation, further indicating that Vps13D regulates mitochondrial clearance in a Pink1-dependent manner.

Pink1 can directly phosphorylate ubiquitin conjugated to proteins at the Ser65 residue, resulting in a conformation change that inhibits de-ubiquitination and can lead to further ubiquitination. The vps13d mutant intestine cells were stained with an antibody specific for ubiquitin phosphorylated at Ser65. Like conjugated ubiquitin, phosphorylated ubiquitin localized around the periphery of mitochondria (labeled by ATP5a) in vps13d mutants but was absent in pink1;vps13d double mutants (FIGS. 17C-17D). These data indicate that pink1 function is required for localization of conjugated and phosphorylated ubiquitin near the perimeter of mitochondria in vps13d mutant cells and indicates that pink1 is upstream of vps13d. To test this, Vps13D protein puncta localization in pink1 mutant intestine cells was investigated. The pink1 mutant cells (lacking RFP) had reduced Vps13D protein puncta compared with neighboring RFP-labeled control cells (FIGS. 17E-17F). Like with loss of core autophagy proteins, loss of Pink1 did not affect Vps13D puncta in early third instar larval intestine cells, indicating that the relationship between Pink1 and Vps13D is stage and context dependent. Taken together, these data indicate that Pink1 and Vps13D can function in a common pathway to regulate mitophagy, with Pink1 acting upstream of Vps13D.

### EQUIVALENTS

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

SEQUENCES
NCBI Reference Sequence Accession Number
NM_015378.4
(SEQ ID NO: 1)
CTGAGCGCCGCGGGCCTGCGCCATTGAGGAGCGGCGGGGAGGAAAC
GCCGCGCAGCGCCGGGCTGGGGCGGGCGGCCCGGGACACCGACAGA
TTTTTCTGTGACCATGAAAGAGAGAAATAAAGAATGATCCATGATT
TCTAAACACCTTTTCCTGAGGATATAGTCATGTTGGAAGGCCTTGT
AGCCTGGGTTCTCAATACCTATTTGGGAAAATATGTCAATAACCTG
AACACTGACCAGCTCTCAGTTGCACTTCTCAAAGGTGCTGTTGAAT
TAGAAAACTTGCCATTAAAGAAAGATGCCTTGAAAGAATTGGAATT
ACCATTTGAAGTCAAAGCTGGCTTCATTGGGAAAGTAACCCTTCAG
ATTCCCTTTTATCGCCCCCATGTGGACCCTTGGGTGATCTCCATCT
CCAGCCTTCACTTAATTGGAGCCCCAGAGAAAATACAGGATTTCAA
TGATGAAAAGGAGAAGCTGTTGGAAAGGGAACGTAAGAAAGCACTA
CTTCAAGCCCTGGAGGAGAAATGGAAGAATGACCGCCAGCAGAAAG
GGGAGTCCTATTGGTATTCAGTTACCGCCTCCGTAGTTACAAGGAT
TGTGGAGAATATTGAATTAAAAATTCAAGATGTCCATTTACGCTTT
GAAGATGGTGTCACCAATCCCTCCCATCCTTTTGCTTTTGGCATCT
GCATTAAGAATGTGTCCATGCAAAATGCTGTGAATGAGCCTGTACA
GAAACTAATGCGGAAAAAGCAATTAGACGTAGCAGAATTTAGCATC
TATTGGGATGTCGATTGCACTTTACTGGGGGATTTGCCTCAGATGG
AGTTACAGGAGGCCATGGCCAGGAGCATGGAGAGTCGCAGCCATCA
CTACGTCCTGGAGCCTGTGTTTGCATCTGCTCTTTTGAAGAGAAAC
TGCTCCAAGAAGCCCCTGCGGTCTCGGCACAGTCCCCGTATTGATT
GTGATATTCAGCTGGAGACCATTCCCTTGAAACTCTCTCAGCTGCA
ATACCGGCAAATCATGGAATTCCTCAAGGAGCTGGAACGAAAGGAG
AGGCAGGTGAAGTTCCGAAGGTGGAAACCCAAGGTGGCGATATCTA
AGAACTGCCGAGAATGGTGGTATTTTGCTTTGAATGCTAACTTGTA
TGAGATCAGAGAGCAGAGGAAACGTTGCACCTGGGACTTTATGTTG
CACCGCGCTCGTGATGCTGTATCTTACACTGACAAATATTTCAACA
AGTTAAAAGGAGGCCTGCTGTCCACAGATGACAAGGAGGAAATGTG
TCGGATTGAAGAGGAACAGAGCTTTGAGGAATTGAAGATTTTGCGT
GAACTGGTTCATGATCGATTTCACAAACAGGAAGAACTAGCAGAGA
GTCTGCGGGAGCCTCAGTTTGATTCTCCAGGAGCCTGTCCGGGAGC
CCCAGAACCCGGTGGAGGCAGTGGGATGCTGCAGTATCTCCAGTCC
TGGTTTCCTGGATGGGGTGGCTGGTACGGGCAGCAGACCCCAGAAG
GGAATGTGGTTGAGGGACTGTCAGCAGAGCAACAGGAGCAGTGGAT
TCCTGAAGAGATCCTGGGCACTGAGGAGTTTTTTGACCCCACTGCA
GATGCCTCGTGTATGAACACGTATACAAAGCGAGATCATGTCTTTG
CCAAACTGAATTTGCAGTTGCAGCGAGGTACAGTGACTCTGTTACA
CAAGGAGCAAGGAACTCCTCAAATGAATGAAAGTGCTTTCATGCAG
CTCGAGTTTTCAGATGTAAAACTTCTAGCAGAGTCTCTTCCTCGAA
GAAATTCCTCGTTGCTTTCAGTCCGGTTGGGTGGACTGTTTCTTCG
AGACCTGGCTACAGAAGGAACTATGTTTCCTCTTCTAGTCTTCCCT
AATCCACAAAAAGAAGTTGGCAGAGTCTCACAATCTTTTGGTCTAC
AAACTACATCTGCAGACAGAAGTGATCATTACCCAGCTGCAGATCC
AGATGGCCCCGTTTTTGAGATGCTGTATGAGAGAAATCCGGCGCAC
AGCCACTTTGAGAGGCGGCTCAATGTCAGCACAAGGCCCTTGAACA
TCATATACAATCCGCAGGCCATTAAAAAAGTAGCAGACTTTTTCTA
CAAGGGAAAGGTTCATACCTCAGGTTTTGGTTATCAGTCTGAACTT
GAGCTGAGAGTGGCTGAAGCTGCCCGAAGACAATATAACAAGCTGA
AGATGCAGACCAAGGCAGAAATCCGGCAAACTCTTGATCGTTTGCT
AGTGGGTGATTTCATTGAGGAGAGTAAACGATGGACCGTGCGGCTG
GATATTTCTGCCCCTCAGGTGATATTTCCTGATGATTTCAAATTCA
AGAATCCTGTGTTAGTTGTCGTGGATCTAGGAAGAATGCTTTTGAC
GAACACCCAAGATAACTCCAGGAGGAAAAGTAGGGATGGGTCAGCA
TCTGAAGAGACCCAGTTTAGTGATGATGAATATAAGACCCCCCTGG
CCACACCTCCTAACACCCCACCTCCCGAGTCAAGCAGCAGCAACGG
AGAGAAAACACCTCCCTTTTCTGGAGTTGAGTTCAGTGAAGAACAG
CTTCAAGCACATTTAATGAGCACAAAGATGTATGAGAGGTACTCGC
TGTCATTTATGGACCTCCAGATCATGGTTGGACGAGTGAAAGACAA
TTGGAAGCATGTCCAGGATATTGACGTGGGACCAACACATGTGGTA
GAGAAGTTCAACGTTCACCTACAGTTAGAGCGTCGATTGATTTATA
CTTCAGATCCCAAATATCCAGGAGCCGTGCTCTCAGGCAACTTACC
AGACTTAAAAATCCACATTAATGAAGATAAAATATCTGCACTAAAG
AATTGCTTTGCTCTCCTCACCACCCCAGAAATGAAAACTTCTGACA
CTCAGATTAAAGAAAAGATTTTTCCCCAGGAGGAGCAGCGGGGAAG
TTTGCAAGACTCCGTAATGAATTTAACCCAGAGCATTGTGTTGTTG
GAGCAGCATACCCGCGAGGTTCTGGTGGAGTCGCAGCTCCTCCTGG
CGGAATTTAAAGTGAACTGTATGCAGCTTGGTGTTGAGAGCAATGG
CCGGTACATTTCTGTGCTCAAGGTGTTTGGTACCAATGCTCACTTT
GTGAAGAGGCCTTATGATGCTGAAGTCTCCCTAACTGTTCATGGTT
TGCTCCTGGTGGATACCATGCAGACATATGGTGCTGATTTTGACCT
TTTGATGGCTTCACATAAGAACTTGAGCTTTGATATTCCAACGGGA
AGCCTTCGGGATAGCAGGGCCCAGTCTCCTGTCTCTGGACCGAATG
TGGCCCACTTAACTGATGGAGCTACACTGAACGACCGATCAGCTAC
TAGTGTTTCACTTGACAAAATTCTTACCAAAGAGCAAGAGTCCCTT
ATTAAGTTGGAATATCAGTTTGTGAGTTCAGAGTGCCCATCGATGA
ATTTAGACAGTACTCTTCAGGTGATTTCCCTACAGGTGAATAATTT
AGATATTATCCTCAATCCAGAGACGATTGTGGAGCTAATTGGTTTT
CTTCAAAAATCCTTTCCCAAGGAAAAAGATGATTTAAGTCCTCAAC
CTTTAATGACTGATTTTGAAAGAAGCTTCAGAGAACAAGGAACTTA
CCAGTCTACATATGAACAAAACACTGAGGTTGCAGTGGAAATCCAT
AGGCTGAACTTACTGCTTCTTCGGACAGTGGGCATGGCAAATAGAG
AGAAATATGGCAGAAAAATTGCAACTGCAAGTATAGGTGGCACCAA
AGTTAATGTCTCAATGGGTAGCACGTTTGACATGAATGGTTCTCTT
GGCTGTTTACAGCTTATGGATTTGACACAAGATAACGTTAAAAACC
AGTATGTTGTCAGCATTGGGAATTCTGTAGGCTATGAAAATATCAT
CAGTGATATTGGCTACTTTGAATCTGTGTTTGTCAGAATGGAAGAT
GCAGCCCTCACTGAAGCTTTGAGTTTCACGTTTGTTGAGAGATCTA
AACAGGAGTGTTTTCTCAACCTGAAGATGGCTTCTTTACATTATAA
CCACTCTGCTAAGTTTTTGAAGGAGTTGACGTTATCCATGGATGAA
CTGGAAGAAAATTTTCGAGGTATGCTGAAAAGCGCAGCCACCAAAG
TCACCACAGTACTAGCTACCAAGACTGCCGAGTATAGCGAGATGGT
ATCGCTCTTTGAAACTCCAAGGAAGACTCGGGAACCCTTTATCTTA
GAGGAAAATGAAATATATGGGTTTGACCTAGCTTCGTCTCATTTGG
ACACTGTAAAGCTAATCTTGAACATAAACATTGAATCACCAGTTGT
TTCTATCCCTCGGAAGCCGGGGAGTCCTGAGTTGTTGGTGGGACAC
TTGGGACAGATATTCATCCAGAATTTTGTGGCGGGAGATGATGAAT
CCAGAAGTGACCGTCTGCAGGTGGAAATCAAGGACATTAAACTGTA
TTCTTTGAATTGCACCCAGTTGGCAGGTAGAGAAGCTGTTGGGTCT
GAAGGAAGCCGGATGTTTTGCCCACCTTCCGGGTCTGGCAGTGCCA
ACAGTCAGGAGGAAGCTCATTTCACACGACATGATTTCTTTGAATC
TTTGCATAGAGGTCAAGCTTTTCACATCCTGAACAACACCACCATT
CAGTTTAAACTGGAGAAGATCCCTATAGAGAGAGAATCTGAATTGA
CTTTTTCTCTTAGCCCAGATGACCTGGGAACTTCTAGCATCATGAA
GATTGAAGGAAAATTTGTCAATCCAGTTCAGGTGGTGTTAGCAAAG
CATGTATATGAGCAGGTTTTACAAACCCTGGACAATCTCGTGTACA
GTGAAGATCTGAATAAGTATCCAGCCAGTGCTACCTCCTCCCCTTG
CCCTGATTCTCCTCTGCCTCCCCTCAGTACCTGTGGAGAATCTTCT
GTTGAAAGGAAGGAGAATGGATTGTTCAGCCACTCCAGCCTTTCTA
ACACCTCTCAGAAGTCATTGTCAGTGAAGGAAGTCAAATCCTTTAC
TCAGATTCAAGCCACCTTTTGTATATCAGAGCTTCAGGTTCAGCTA
AGTGGAGATCTGACTTTGGGGGCCCAAGGTCTTGTGAGCTTAAAGT
TTCAGGACTTTGAGGTGGAATTCAGTAAAGACCATCCCCAGACTTT
ATCTATTCAGATTGCCCTGCATTCTCTGCTGATGGAGGACTTATTG
GAGAAGAATCCAGATTCTAAATATAAGAACCTGATGGTGTCTCGAG
GAGCCCCTAAGCCATCTAGTTTAGCACAAAAAGAATACCTTTCTCA
GTCTTGCCCCTCAGTGTCCAATGTGGAATATCCTGATATGCCTCGG
TCTCTCCCTTCCCACATGGAAGAAGCTCCTAATGTCTTCCAGTTGT
ATCAAAGGCCCACCTCTGCGTCCCGGAAAAAGCAAAAGGAAGTCCA
AGACAAGGACTATCCCTTGACCCCACCTCCTTCTCCAACAGTGGAT
GAGCCCAAGATACTTGTTGGAAAGAGTAAATTTGATGATTCCTTAG
TCCACATCAACATATTCTTGGTAGATAAGAAACATCCAGAATTCTC
TTCCAGTTACAATCGAGTTAACCGGAGCATTGATGTTGATTTTAAT
TGCTTGGATGTGCTGATCACACTGCAAACCTGGGTTGTGATATTAG
ACTTTTTTGGAATCGGCTCCACTGCAGACAACCACGCAATGAGGCT
GCCTCCTGAGGGCATTCTGCACAACGTGAAGTTGGAGCCACATGCC
TCCATGGAGTCTGGACTTCAGGATCCAGTGAACACCAAACTGGATC
TCAAGGTTCATTCACTTTCTCTAGTGCTGAATAAGACCACCAGTGA
GCTTGCCAAAGCAAATGTGTCCAAATTAGTAGCACACCTGGAAATG
ATTGAGGGAGACCTGGCCTTACAGGGCAGCATTGGGAGTCTGTCTC
TAAGTGACCTCACATGCCATGGAGAGTTCTACAGAGAACGGTTCAC
TACCAGTGGTGAAGAAGCACTCATCTTCCAGACTTTTAAATACGGA
CGGCCTGACCCTCTGCTCCGGAGAGAACACGACATTCGCGTGAGCC
TCCGGATGGCCTCTGTGCAGTATGTGCATACTCAGCGTTTCCAGGC
AGAGGTGGTGGCCTTCATTCAGCATTTCACTCAGCTGCAGGATGTC
TTAGGGCGCCAGCGAGCTGCTATTGAGGGGCAGACGGTGAGAGATC
AAGCCCAGCGCTGTTCACGGGTTCTCCTGGATATTGAGGCTGGTGC
TCCCGTTCTCTTGATCCCAGAAAGTTCCAGATCAAATAATCTGATT
GTAGCAAATTTGGGGAAGTTGAAAGTCAAAAATAAGTTTCTGTTTG
CTGGTTTTCCTGGCACCTTTTCCCTACAAGATAAGGAATCTGTGCC
TTCAGCTTCCCCAACGGGTATTCCCAAACACAGTCTGAGGAAAACG
ACAAGCACGGAGGAGCCCAGGGGAACCCATTCCCAGGGGCAGTTCA
CGATGCCTCTTGCTGGAATGAGCCTAGGAAGCCTGAAGAGTGAGTT
TGTGCCCAGTACCTCCACCAAGCAGCAAGGGCCGCAACCCACACTG
TCTGTTGGCCAAGAGTCCAGTAGTCCAGAAGACCATGTCTGCCTGC
TGGATTGCGTTGTCGTGGATCTCCAGGACATGGACATCTTTGCTGC
AGAGAGACATCCGAGAGAATACTCGAAGGCACCAGAGGATAGTAGT
GGAGATCTGATCTTCCCTTCCTATTTTGTGCGACAGACAGGAGGAA
GCCTCTTAACCGAGCCTTGTAGGCTGAAATTGCAGGTGGAAAGGAA
TTTGGACAAAGAAATAAGTCATACTGTGCCAGACATATCTATCCAT
GGCAATCTCTCCTCAGTCCACTGCTCTCTGGATCTGTATAAATACA
AGCTGATCCGCGGCTTATTAGAGAACAACCTGGGAGAACCCATAGA
GGAATTTATGCGGCCTTATGATTTACAAGATCCAAGAATTCATACT
GTCCTGAGTGGAGAAGTGTACACCTGTATGTGCTTCCTCATTGATA
TGGTGAATGTAAGTCTGGAGCTTAAAGATCCAAAAAGAAAAGAAGG
TGCTGGGTCCCTAGCCAGATTTGACTTCAAGAAATGCAAACTGCTC
TATGAAAGTTTTTCCAACCAAACCAAGTCCATTAACTTGGTTTCCC
ATTCCATGATGGCTTTTGACACCCGTTATGCTGGGCAGAAGACCAG
CCCTGGCATGACGAATGTGTTCAGCTGTATCTTTCAGCCCGCTAAG
AACAGCAGCACCACCCAAGGGTCCATTCAGATTGAACTACATTTCA
GATCTACCAAGGATTCCTCCTGCTTTACAGTAGTTCTCAACAATCT
CCGTGTGTTTCTCATATTTGACTGGCTACTGTTAGTCCATGATTTT
CTCCACACTCCCAGTGATATTAAGAAACAAAATCATGTTACTCCTT
CTCGCCACCGTAACTCTAGCAGCGAATCTGCTATAGTTCCCAAAAC
TGTGAAGAGTGGAGTAGTTACCAAGCGGTCTTCCCTTCCTGTGTCC
AATGAAAGGCACCTGGAGGTCAAGGTCAATGTAACAGGTACGGAGT
TTGTGGTCATTGAAGATGTGTCCTGCTTCGACACCAATGCCATTAT
TCTGAAAGGCACCACAGTGCTCACCTATAAGCCCCGGTTTGTTGAT
CGCCCCTTTTCAGGAAGTTTGTTTGGCATTGAGGTGTTTTCATGCC
GACTAGGGAATGAGCATGATACAGCTCTTTCAATTGTGGATCCCGT
ACAAATTCAAATGGAGTTGGTGGGGAATTCTTCTTATCAAAATAGT
TCAGGATTGATGGATGCATTCAATAGTGAAGATTTCCCACCTGTCC
TGGAGATTCAGTTACAAGCCCTGGATATCAGACTCTCCTATAATGA
TGTTCAGCTGTTTCTTGCCATTGCAAAATCCATCCCAGAGCAAGCT
AATGCTGCAGTGCCAGACTCAGTGGCCCTGGAGTCAGACTCCGTTG
GCACTTACCTTCCAGGTGCATCTCGCGTTGGAGAGGAAATCAGAGA
AGGGACAAGACACACCTTAGATCCTGTCTTGGAGTTACAGCTGGCT
AGGCTGCAGGAGCTGGGATTCAGCATGGATGATTGTCGCAAAGCTC
TTTTGGCGTGTCAAGGCCAATTGAAAAAGGCAGCAAGTTGGTTGTT
TAAGAATGCGGAACCTCTGAAGTCTCTTTCCTTGGCCTCCACCAGC
CGAGATAGCCCAGGGGCTGTGGCAGCGCCATTGATCTCTGGCGTGG
AGATCAAAGCTGAGAGTGTGTGCATCTGTTTCATCGATGACTGCAT
GGATTGTGATGTTCCTCTCGCTGAACTCACCTTTTCCCGTCTGAAT
TTTCTTCAGCGTGTAAGAACTAGCCCTGAAGGCTATGCCCACTTCA
CCCTTTCTGGAGATTATTATAACCGTGCTCTTTCAGGCTGGGAGCC
ATTTATTGAGCCTTGGCCATGCTCTGTATCCTGGCAACAGCAGGCA
GCTAGTCGTCTCCATCCTCCTCGACTGAAGCTAGAAGCCAAGGCCA
AACCTCGTTTGGATATCAATATCACTTCTGTGCTAATTGACCAGTA
TGTAAGTACCAAGGAATCGTGGATGGCAGATTACTGTAAAGATGAC
AAGGACATAGAGTCAGCTAAATCAGAAGACTGGATGGGCTCTTCGG
TGGATCCTCCATGTTTTGGACAAAGCCTCCCCCTTGTCTACCTTAG
AACTAGGAGTACAGCCAGTCTGACTAACCTAGAGCACCAGATCTAT
GCTAGAGCAGAGGTGAAAACCCCCAAGCGCCGGCAGCCATTTGTCC
CCTTTGCTCTGAGGAACCACACGGGGTGCACTTTGTGGTTTGCCAC
CCTGACCACCACACCCACCAGAGCTGCACTCTCTCACAGTGGGAGT
CCAGGGGTAGTTCCAGAAGGGAACGGAACATTTCTCGATGATACTC
ACAATGTTAGTGAATGGCGAGAAGTCCTTACAGGTGAAGAGATTCC
CTTTGAATTTGAAGCAAGAGGAAAGTTAAGACACAGACACACCCAT
GACCTCCGGATTCATCAACTGCAAGTGAGAGTAAATGGCTGGGAGC
AAGTGAGCCCAGTGTCTGTGGACAAAGTCGGGACCTTTTTTCGATA
TGCAGCACCAGATAAAAATTCATCTTCCTCTACGATTGGCAGCCCA
AGCAGCAGAACAAATATTATACATCCCCAGGTTTATTTCTCTTCAC
TCCCACCAGTGCGGGTGGTCTTTGCAGTGACTATGGAAGGCAGTGC
ACGGAAAGTCATCACTGTCCGGTCAGCCCTCATTGTGAGGAACAGA
CTTGAGACACCAATGGAACTAAGACTGGATAGCCCATCAGCTCCAG
ACAAGCCAGTGGTGCTTCCTGCTATCATGCCAGGGGATTCGTTTGC
TGTGCCTTTACACCTCACTTCTTGGCGGCTACAGGCCCGGCCCAAA
GGATTGGGTGTATTTTTCTGTAAGGCTCCCATTCATTGGACCAATG
TAGTGAAGACTGCAGAAATTAGTAGCAGTAAACGAGAGTGCCACTC
TATGGACACAGAAAAAAGCCGATTTTTCAGGTTTTGTGTGGCTATA
AAGAAAGAGAATTATCCAGATTATATGCCCTCAAACATATTTTCTG
ACAGTGCAAAACAGATTTTCAGACAGCCTGGGCATACCATATATCT
CCTGCCAACTGTGGTAATCTGCAACTTGCTACCCTGTGAACTTGAT
TTTTATGTTAAAGGAATGCCAATTAATGGGACGCTGAAACCTGGCA
AGGAGGCAGCTCTCCATACAGCTGATACATCCCAGAACATTGAGCT
GGGGGTATCACTGGAGAATTTCCCCCTCTGTAAAGAATTGCTCATT
CCACCTGGAACCCAAAACTATATGGTGAGAATGCGACTCTATGACG
TCAACCGTCGGCAGCTGAACCTCACCATCCGGATTGTGTGTCGAGC
AGAAGGATCCTTAAAGATCTTCATTTCTGCTCCATATTGGCTGATT
AACAAAACAGGGTTGCCACTGATCTTCAGACAGGACAATGCCAAGA
CAGATGCTGCAGGCCAGTTTGAGGAGCATGAGCTGGCCCGTAGCCT
GAGTCCTCTCTTATTCTGCTATGCTGACAAAGAGCAGCCAAACCTC
TGCACGATGAGAATCGGAAGGGGGATTCATCCAGAAGGCATGCCGG
GCTGGTGTCAGGGCTTCTCCCTGGATGGTGGTAGTGGTGTCCGAGC
TTTGAAAGTCATCCAGCAAGGAAACCGCCCAGGGCTGATCTATAAC
ATTGGTATTGATGTCAAGAAAGGCCGAGGTCGATACATTGATACCT
GCATGGTCATCTTTGCCCCCCGTTACCTGTTAGATAATAAATCATC
TCACAAGCTTGCATTTGCACAGAGGGAATTTGCCAGGGGACAGGGA
ACAGCCAATCCCGAAGGTTACATTTCCACCCTTCCTGGTTCCAGTG
TGGTGTTCCACTGGCCTCGGAATGACTATGATCAGCTATTGTGTGT
CAGACTGATGGACGTTCCCAATTGTATTTGGTCTGGAGGCTTTGAA
GTCAACAAGAATAATTCCTTCCATATCAACATGAGGGATACCTTGG
GAAAATGCTTCTTCCTACGAGTGGAAATTACTCTCCGAGGAGCTAC
GTATAGGATCTCATTTAGTGACACAGATCAGTTACCTCCTCCTTTC
CGAATTGACAACTTTTCTAAGGTCCCGGTTGTCTTTACTCAGCATG
GCGTAGCTGAACCCAGGCTCCGGACTGAAGTGAAGCCCATGACTTC
ATTGGATTATGCCTGGGACGAACCCACCTTGCCACCTTTTATCACT
CTGACTGTTAAAGGGGCAGGGTCCTCTGAGATCAACTGCAACATGA
ATGATTTCCAGGATAATCGGCAGCTTTATTATGAAAATTTCATTTA
CATTGCTGCTACATATACATTCTCTGGCTTGCAGGAGGGAACAGGC
AGGCCTGTGGCTTCCAACAAGGCCATTACCTGTGCGGAGCTCGTTT
TGGATGTCTCACCCAAGACACAAAGAGTCATTTTAAAAAAGAAGGA
ACCAGGAAAGCGTTCTCAGCTGTGGAGGATGACAGGAACAGGAATG
CTGGCCCATGAGGGCTCCTCAGTTCCTCACAATCCCAATAAGCCCT
CAGCCGCCCGCTCCACCGAGGGGTCTGCCATCTTAGATATTGCTGG
TCTCGCTGCAGTGACTGACAACAGATACGAGCCACTGATGCTGAGA
AAGCCTGACCGCAGGCGAAGCACAACTCAGACGTGGAGTTTCCGAG
AAGGAAAACTGACCTGTGGGTTACATGGGTTGGTCGTCCAGGCCAA
AGGAGGACTTTCTGGTTTGTTTGATGGAGCTGAAGTTGTTCTTGGT
CCTGACACTTCCATGGAGCTTTTGGGGCCAGTTCCACCTGAACAAC
AATTTATTAATCAAAAAATGAGACCTGGTTCTGGAATGTTATCCAT
CAGAGTCATCCCAGATGGACCAACTAGAGCACTCCAGATAACAGAT
TTCTGCCACCGGAAAAGCAGCCGTTCATATGAAGTGGATGAACTTC
CTGTCACCGAACAAGAGCTGCAGAAATTAAAGAATCCAGATACAGA
GCAGGAATTGGAAGTGCTTGTGAGGTTAGAAGGTGGAATTGGGTTG
TCCTTAATTAATAAAGTCCCAGAAGAACTGGTCTTTGCAAGTCTTA
CAGGAATCAATGTGCACTATACACAGCTGGCAACCAGTCACATGCT
TGAACTCAGCATACAGGATGTACAGGTGGACAATCAGCTCATTGGT
ACCACGCAGCCCTTCATGCTCTATGTGACTCCCCTGAGCAATGAGA
ATGAGGTCATCGAGACCGGCCCAGCTGTGCAAGTCAACGCAGTGAA
GTTCCCCAGTAAGAGTGCACTGACCAACATCTACAAGCATCTGATG
ATCACAGCTCAGAGATTCACAGTGCAAATTGAGGAGAAACTGCTCC
TCAAGCTGCTAAGTTTCTTTGGCTACGATCAAGCAGAATCAGAGGT
GGAAAAATATGATGAAAACCTCCATGAAAAGACAGCTGAGCAAGGT
GGAACACCAATTCGATACTACTTTGAAAATCTCAAAATCAGCATTC
CTCAGATCAAGCTAAGTGTGTTCACCTCCAACAAGCTCCCATTGGA
TCTTAAGGCCCTAAAAAGCACCTTGGGGTTTCCTTTGATACGGTTT
GAAGACGCTGTGATTAATCTAGATCCATTCACTCGGGTACATCCCT
ATGAGACCAAGGAGTTCATCATCAATGATATCCTCAAACATTTCCA
GGAGGAACTCCTCAGCCAGGCAGCTCGAATCCTGGGATCAGTGGAT
TTTCTTGGCAATCCTATGGGGCTTTTGAATGATGTTTCTGAAGGGG
TTACTGGACTGATAAAATATGGAAATGTCGGGGGCCTCATCAGAAA
TGTTACACACGGAGTATCAAACTCTGCTGCCAAGTTTGCTGGAACA
TTATCAGATGGCTTAGGGAAGACGATGGACAATCGGCATCAGTCAG
AGCGGGAGTACATCAGGTACCATGCAGCCACAAGTGGTGAACACCT
TGTAGCCGGCATCCATGGCCTGGCTCATGGTATCATTGGTGGACTG
ACCAGTGTTATAACTTCGACAGTGGAAGGTGTGAAAACAGAAGGGG
GTGTCAGCGGTTTCATATCTGGCCTTGGAAAAGGGCTTGTTGGCAC
TGTAACCAAGCCAGTGGCAGGCGCCCTGGATTTTGCATCAGAAACA
GCCCAGGCGGTGAGAGACACAGCCACACTCAGCGGCCCCAGGACTC
AAGCACAGAGGGTTCGGAAACCGCGTTGCTGCACGGGGCCCCAGGG
GCTGCTTCCCCGATATTCTGAGAGCCAGGCGGAAGGACAGGAGCAG
CTCTTCAAACTCACAGACAACATACAGGACGAATTCTTCATCGCTG
TGGAGAACATTGACAGCTACTGCGTGCTCATCTCCTCCAAAGCTGT
TTACTTCCTGAAAAGTGGAGACTACGTGGATCGAGAAGCCATTTTC
CTAGAAGTCAAATACGATGACCTCTACCACTGCCTTGTCTCCAAAG
ACCATGGGAAGGTGTATGTGCAGGTGACCAAGAAAGCCGTGAGCAC
GAGCAGTGGAGTGTCCATCCCCGGCCCCTCCCACCAGAAGCCCATG
GTCCATGTGAAATCTGAGGTCCTTGCTGTCAAGTTGTCACAAGAAA
TAAACTACGCAAAGAGCCTCTACTATGAACAGCAGCTTATGTTAAG
ACTCAGCGAAAACCGAGAGCAGCTGGAGCTGGACTCCTGAAGCCCC
GCTGCTGAGATGGGCGCTCCCGACACAGCGCAGACCCACCAGGAGG
AAAGAGGCCCAGCTCTCAGCTGACGATGGAGGCAGAACCGGAGTCG
GGTTTGGGGAAGTTGTCAAGGAATGAGGGAAAGTAAATCCTCATGA
GGAAAAGTACAAATGGAAATCGTATTAATTTGTGAGGCAGGGAGTT
ATTTTAGATTATGGGAAATAATTTTTAAAGGTATTGGTTAAATAAC
GTTTAAAAACATGTACTGAGATGAATCTAATTTTTAGATTGCCCTG
TATTTTGTTAACATGTATATATGTACAACAGTGTGTTTGTAAATAT
ATAGGAACGTTTCTGAACAGGGTCTGTGCTATGTGTAAAGGTTTGT
TAACTGTAAAGTAATATAAAGTTATATTGGATCTTCTATTGCACTA
ATTCTAGATGTCTAATTCAGGATACTGTCTATAGAAAGGCATTCTT
AAAAGTTAAAGAATGTTACGTCTTAGTTTTGGAGACTAAAGTATTC
CCAGTAAAGTGGGTTGAGGTGAGGGCTGTGGTCCTGAAAGGGACGC
CTTTGACATCGTGGCTGTCCAGTTGGGCTGTGAGCTGTGGCACCCA
GGACTGGCGCTGGCCCTTCAGAAGGATCTAGGAGAGGGGCTTGGGA
GCCCACTTTTAATTTCTCACCCCCATTTTACAAAGAGTGCTTAGAT
TCTTACAAATTATGATGTAAGTTATCCATTTGGCTTTTTCCTAACT
AGTCTTACCAAACTTAGGGGGAAACCTGTGCTCCATTACCACATGG
GTGCAAGTCAGCATTGTAAGTTTTCTCAGGTTATTATTATTAGAGA
GGTTGGAAACATTGGTAAACTCTGTTGATTGAGAAGGAAAAAAAAA
GTCCCATTGAACTGTTGCAACAAATCAGAAATCCACATAAAAGTGC
TCTCCTGCCTGGGCAGCAACAACCAAGAACAAAGCCCCGGGACTGT
TTTCTTTTTAATAAAGCCACAGGCAGGCATCGTAGCTCCACAGCCC
GAGGGGACACAGGATGGAAACCCCAGGATGAGAAGGGAGCAGGGAG
AGTTCCAGAAAGGGGGATGAAATAGGAGTATTAAAAAGCTGCGTTG
GTAAGTTTTTCATGGAACCAAGATTTGACAAAGGCATCTCTTATCC
TTGGTTTTAAATTCCTGCTGGGAGCAAGGCCTGGTATGAGCGCCCT
GGGTCTTGTTTTTGGTGTTTCGCTTTTCTGTAAGGATTAAGCAGAT
AGGGAGAAGGGAAAAGGGGCCTCACTTTAGAATGAATGAGTCACCT
TGTGATTTTTAAATTTTTATTTTAATAAAGCTAATCAATTTCTACA
ACCTTGTCACATGTAGCTGAGTCTGGGATGACTCAGTGGATCAGTG
GATGCGTGGAAGGTTTTGGTGTTTATAACTCATGACCCAAATCCTT
CCAAGACACAAATGAAGCTAATCTTCTTCCCCTCCCTTTCCTCTCC
CACTTCCCCTCTGTGTCTGTGTCTGTGTGTGTGTGTGTGTGTGTGT
GTGTGTGCGCGCGCGCGCGTGCATGCAGAGAGGAAGGAAGGGAGCT
TTAGCACAAGTAGCTACATAGTGTTCAAGGATCAGCTTTCTTGAAC
TCCGAGGCTTGGTTGCCTTTGCTGAACTCCAAGTTAGTGGATTGCA
GAATGGAAACTTGGCTTTTGCGGCACTGGGTGAGTTTTAGTTTGTG
TGTGTCTTGCTGGGGGGTGGTGATGATTGTCTCAGCACTCACGCAC
TGCACAAGATGGCAGCAGGATACAGCACTGCACAAGATGGCAGCTC
CTCTGCAGCTTCCTCCTCAGCCTCCCTCCTTGCACCCCCACAGGTT
TGGCTTGTGGTTTTTGTCATCAGTAACCTACTGCCTGAGATCATGA
TCTCTTAAAAGATGAGACTCTCGGAAGGGTTGATTGTATGCGTCAG
TGAGCCTTCTATCACCTTCTGGAACAAAGTCACTTGAAATCTCTTG
ATGAGATTAAGGAGTTTAGTGTTACTAAGAAAATCTGCTTTGGGCC
GCAGCAGTGCTGGGTGTTCTCAGACCTGACTGAGGAAGTTAGCTGC
GGGCTGCCCTGTGGGCTGGTGCTTCAGGAGGAATCCAGAGAAGTGT
TCAGATGCCCCCCTTGGGCTCCTTTCTAATTTTAATCAGCTCTTTA
AATAGCTGCCCATCTCCTGTGATTGCACAACCAAGCACTTTGACAT
TTGCACCTTAGGAGAGGCAGATGTTAAAATGGAATCCAAAGACCAC
CTAGGGCGGGGCTGGGTGGGAGATGGGAGGGCCAACTGCGAGCTGC
TCCACTTCTCAGCTCTCCCCTGCCCTGCAGCCCTGGGCCAGACAAG
GCCAGAAGGTTTCAGGGGCATTTGACATCCCCTCCTGGTTCTCACC
AGGAAAACATCCAAAGCTTTGGAGGAAACAGGCCCTGCCCCTGGCT
CCTTAAATGCCCCGTCTCTTTGTAAACTGATATTCAGCCAGCAATG
CCTAAGACTTTGTTAAGATCATTTCTACTGCTTTTCTTTCTGCTTC
AAACACACAGTTCGTCTCTGAGGAAAGTAAAATAAATGGAATAAGA
GTAAATTGGGTAAGGAGATATCCAAAGCTACCCAGTCCCTTGACCC
AGCACAGTTGGCCGACCCGTGTCACTCCCTGGCTGTCGCTGCTTCT
CTGTGCTCACTGAAGGGTGAGCCAGGCCAGTGCTTCCCCAGCCCCT
GGGCCTGGTCACTACACAGTGGAAAACAGACAAGCGGCCCCTTCCC
CAAATCCCAAGAGTGTCTTGCTGCTTGGTGGGTGCTCATCGCAATG
TTCTGAAGGCTCCAGGGCCACTTTGTTTGTAAGTATGATCTGGGCC
TCAAAATACCATAGTAGCTGCTTGATAAAATTCTAAAAATATCTGG
TTCTCTATTATGTAAACACTATTACAGTCACCAGTGTGTGAAGACT
CTTGAGTCTGGTTCTCATATCAGAGTCATCATTTTTCTTCCTGTGG
AATAAAATGCCTTGTGGACTTCCCAAA
NCBI Reference Sequence Accession Number
NM_018156.4
(SEQ ID NO: 2)
CTGAGCGCCGCGGGCCTGCGCCATTGAGGAGCGGCGGGGAGGAAAC
GCCGCGCAGCGCCGGGCTGGGGCGGGCGGCCCGGGACACCGACAGA
TTTTTCTGTGACCATGAAAGAGAGAAATAAAGAATGATCCATGATT
TCTAAACACCTTTTCCTGAGGATATAGTCATGTTGGAAGGCCTTGT
AGCCTGGGTTCTCAATACCTATTTGGGAAAATATGTCAATAACCTG
AACACTGACCAGCTCTCAGTTGCACTTCTCAAAGGTGCTGTTGAAT
TAGAAAACTTGCCATTAAAGAAAGATGCCTTGAAAGAATTGGAATT
ACCATTTGAAGTCAAAGCTGGCTTCATTGGGAAAGTAACCCTTCAG
ATTCCCTTTTATCGCCCCCATGTGGACCCTTGGGTGATCTCCATCT
CCAGCCTTCACTTAATTGGAGCCCCAGAGAAAATACAGGATTTCAA
TGATGAAAAGGAGAAGCTGTTGGAAAGGGAACGTAAGAAAGCACTA
CTTCAAGCCCTGGAGGAGAAATGGAAGAATGACCGCCAGCAGAAAG
GGGAGTCCTATTGGTATTCAGTTACCGCCTCCGTAGTTACAAGGAT
TGTGGAGAATATTGAATTAAAAATTCAAGATGTCCATTTACGCTTT
GAAGATGGTGTCACCAATCCCTCCCATCCTTTTGCTTTTGGCATCT
GCATTAAGAATGTGTCCATGCAAAATGCTGTGAATGAGCCTGTACA
GAAACTAATGCGGAAAAAGCAATTAGACGTAGCAGAATTTAGCATC
TATTGGGATGTCGATTGCACTTTACTGGGGGATTTGCCTCAGATGG
AGTTACAGGAGGCCATGGCCAGGAGCATGGAGAGTCGCAGCCATCA
CTACGTCCTGGAGCCTGTGTTTGCATCTGCTCTTTTGAAGAGAAAC
TGCTCCAAGAAGCCCCTGCGGTCTCGGCACAGTCCCCGTATTGATT
GTGATATTCAGCTGGAGACCATTCCCTTGAAACTCTCTCAGCTGCA
ATACCGGCAAATCATGGAATTCCTCAAGGAGCTGGAACGAAAGGAG
AGGCAGGTGAAGTTCCGAAGGTGGAAACCCAAGGTGGCGATATCTA
AGAACTGCCGAGAATGGTGGTATTTTGCTTTGAATGCTAACTTGTA
TGAGATCAGAGAGCAGAGGAAACGTTGCACCTGGGACTTTATGTTG
CACCGCGCTCGTGATGCTGTATCTTACACTGACAAATATTTCAACA
AGTTAAAAGGAGGCCTGCTGTCCACAGATGACAAGGAGGAAATGTG
TCGGATTGAAGAGGAACAGAGCTTTGAGGAATTGAAGATTTTGCGT
GAACTGGTTCATGATCGATTTCACAAACAGGAAGAACTAGCAGAGA
GTCTGCGGGAGCCTCAGTTTGATTCTCCAGGAGCCTGTCCGGGAGC
CCCAGAACCCGGTGGAGGCAGTGGGATGCTGCAGTATCTCCAGTCC
TGGTTTCCTGGATGGGGTGGCTGGTACGGGCAGCAGACCCCAGAAG
GGAATGTGGTTGAGGGACTGTCAGCAGAGCAACAGGAGCAGTGGAT
TCCTGAAGAGATCCTGGGCACTGAGGAGTTTTTTGACCCCACTGCA
GATGCCTCGTGTATGAACACGTATACAAAGCGAGATCATGTCTTTG
CCAAACTGAATTTGCAGTTGCAGCGAGGTACAGTGACTCTGTTACA
CAAGGAGCAAGGAACTCCTCAAATGAATGAAAGTGCTTTCATGCAG
CTCGAGTTTTCAGATGTAAAACTTCTAGCAGAGTCTCTTCCTCGAA
GAAATTCCTCGTTGCTTTCAGTCCGGTTGGGTGGACTGTTTCTTCG
AGACCTGGCTACAGAAGGAACTATGTTTCCTCTTCTAGTCTTCCCT
AATCCACAAAAAGAAGTTGGCAGAGTCTCACAATCTTTTGGTCTAC
AAACTACATCTGCAGACAGAAGTGATCATTACCCAGCTGCAGATCC
AGATGGCCCCGTTTTTGAGATGCTGTATGAGAGAAATCCGGCGCAC
AGCCACTTTGAGAGGCGGCTCAATGTCAGCACAAGGCCCTTGAACA
TCATATACAATCCGCAGGCCATTAAAAAAGTAGCAGACTTTTTCTA
CAAGGGAAAGGTTCATACCTCAGGTTTTGGTTATCAGTCTGAACTT
GAGCTGAGAGTGGCTGAAGCTGCCCGAAGACAATATAACAAGCTGA
AGATGCAGACCAAGGCAGAAATCCGGCAAACTCTTGATCGTTTGCT
AGTGGGTGATTTCATTGAGGAGAGTAAACGATGGACCGTGCGGCTG
GATATTTCTGCCCCTCAGGTGATATTTCCTGATGATTTCAAATTCA
AGAATCCTGTGTTAGTTGTCGTGGATCTAGGAAGAATGCTTTTGAC
GAACACCCAAGATAACTCCAGGAGGAAAAGTAGGGATGGGTCAGCA
TCTGAAGAGACCCAGTTTAGTGATGATGAATATAAGACCCCCCTGG
CCACACCTCCTAACACCCCACCTCCCGAGTCAAGCAGCAGCAACGG
AGAGAAAACACCTCCCTTTTCTGGAGTTGAGTTCAGTGAAGAACAG
CTTCAAGCACATTTAATGAGCACAAAGATGTATGAGAGGTACTCGC
TGTCATTTATGGACCTCCAGATCATGGTTGGACGAGTGAAAGACAA
TTGGAAGCATGTCCAGGATATTGACGTGGGACCAACACATGTGGTA
GAGAAGTTCAACGTTCACCTACAGTTAGAGCGTCGATTGATTTATA
CTTCAGATCCCAAATATCCAGGAGCCGTGCTCTCAGGCAACTTACC
AGACTTAAAAATCCACATTAATGAAGATAAAATATCTGCACTAAAG
AATTGCTTTGCTCTCCTCACCACCCCAGAAATGAAAACTTCTGACA
CTCAGATTAAAGAAAAGATTTTTCCCCAGGAGGAGCAGCGGGGAAG
TTTGCAAGACTCCGTAATGAATTTAACCCAGAGCATTGTGTTGTTG
GAGCAGCATACCCGCGAGGTTCTGGTGGAGTCGCAGCTCCTCCTGG
CGGAATTTAAAGTGAACTGTATGCAGCTTGGTGTTGAGAGCAATGG
CCGGTACATTTCTGTGCTCAAGGTGTTTGGTACCAATGCTCACTTT
GTGAAGAGGCCTTATGATGCTGAAGTCTCCCTAACTGTTCATGGTT
TGCTCCTGGTGGATACCATGCAGACATATGGTGCTGATTTTGACCT
TTTGATGGCTTCACATAAGAACTTGAGCTTTGATATTCCAACGGGA
AGCCTTCGGGATAGCAGGGCCCAGTCTCCTGTCTCTGGACCGAATG
TGGCCCACTTAACTGATGGAGCTACACTGAACGACCGATCAGCTAC
TAGTGTTTCACTTGACAAAATTCTTACCAAAGAGCAAGAGTCCCTT
ATTAAGTTGGAATATCAGTTTGTGAGTTCAGAGTGCCCATCGATGA
ATTTAGACAGTACTCTTCAGGTGATTTCCCTACAGGTGAATAATTT
AGATATTATCCTCAATCCAGAGACGATTGTGGAGCTAATTGGTTTT
CTTCAAAAATCCTTTCCCAAGGAAAAAGATGATTTAAGTCCTCAAC
CTTTAATGACTGATTTTGAAAGAAGCTTCAGAGAACAAGGAACTTA
CCAGTCTACATATGAACAAAACACTGAGGTTGCAGTGGAAATCCAT
AGGCTGAACTTACTGCTTCTTCGGACAGTGGGCATGGCAAATAGAG
AGAAATATGGCAGAAAAATTGCAACTGCAAGTATAGGTGGCACCAA
AGTTAATGTCTCAATGGGTAGCACGTTTGACATGAATGGTTCTCTT
GGCTGTTTACAGCTTATGGATTTGACACAAGATAACGTTAAAAACC
AGTATGTTGTCAGCATTGGGAATTCTGTAGGCTATGAAAATATCAT
CAGTGATATTGGCTACTTTGAATCTGTGTTTGTCAGAATGGAAGAT
GCAGCCCTCACTGAAGCTTTGAGTTTCACGTTTGTTGAGAGATCTA
AACAGGAGTGTTTTCTCAACCTGAAGATGGCTTCTTTACATTATAA
CCACTCTGCTAAGTTTTTGAAGGAGTTGACGTTATCCATGGATGAA
CTGGAAGAAAATTTTCGAGGTATGCTGAAAAGCGCAGCCACCAAAG
TCACCACAGTACTAGCTACCAAGACTGCCGAGTATAGCGAGATGGT
ATCGCTCTTTGAAACTCCAAGGAAGACTCGGGAACCCTTTATCTTA
GAGGAAAATGAAATATATGGGTTTGACCTAGCTTCGTCTCATTTGG
ACACTGTAAAGCTAATCTTGAACATAAACATTGAATCACCAGTTGT
TTCTATCCCTCGGAAGCCGGGGAGTCCTGAGTTGTTGGTGGGACAC
TTGGGACAGATATTCATCCAGAATTTTGTGGCGGGAGATGATGAAT
CCAGAAGTGACCGTCTGCAGGTGGAAATCAAGGACATTAAACTGTA
TTCTTTGAATTGCACCCAGTTGGCAGGTAGAGAAGCTGTTGGGTCT
GAAGGAAGCCGGATGTTTTGCCCACCTTCCGGGTCTGGCAGTGCCA
ACAGTCAGGAGGAAGCTCATTTCACACGACATGATTTCTTTGAATC
TTTGCATAGAGGTCAAGCTTTTCACATCCTGAACAACACCACCATT
CAGTTTAAACTGGAGAAGATCCCTATAGAGAGAGAATCTGAATTGA
CTTTTTCTCTTAGCCCAGATGACCTGGGAACTTCTAGCATCATGAA
GATTGAAGGAAAATTTGTCAATCCAGTTCAGGTGGTGTTAGCAAAG
CATGTATATGAGCAGGTTTTACAAACCCTGGACAATCTCGTGTACA
GTGAAGATCTGAATAAGTATCCAGCCAGTGCTACCTCCTCCCCTTG
CCCTGATTCTCCTCTGCCTCCCCTCAGTACCTGTGGAGAATCTTCT
GTTGAAAGGAAGGAGAATGGATTGTTCAGCCACTCCAGCCTTTCTA
ACACCTCTCAGAAGTCATTGTCAGTGAAGGAAGTCAAATCCTTTAC
TCAGATTCAAGCCACCTTTTGTATATCAGAGCTTCAGGTTCAGCTA
AGTGGAGATCTGACTTTGGGGGCCCAAGGTCTTGTGAGCTTAAAGT
TTCAGGACTTTGAGGTGGAATTCAGTAAAGACCATCCCCAGACTTT
ATCTATTCAGATTGCCCTGCATTCTCTGCTGATGGAGGACTTATTG
GAGAAGAATCCAGATTCTAAATATAAGAACCTGATGGTGTCTCGAG
GAGCCCCTAAGCCATCTAGTTTAGCACAAAAAGAATACCTTTCTCA
GTCTTGCCCCTCAGTGTCCAATGTGGAATATCCTGATATGCCTCGG
TCTCTCCCTTCCCACATGGAAGAAGCTCCTAATGTCTTCCAGTTGT
ATCAAAGGCCCACCTCTGCGTCCCGGAAAAAGCAAAAGGAAGTCCA
AGACAAGGACTATCCCTTGACCCCACCTCCTTCTCCAACAGTGGAT
GAGCCCAAGATACTTGTTGGAAAGAGTAAATTTGATGATTCCTTAG
TCCACATCAACATATTCTTGGTAGATAAGAAACATCCAGAATTCTC
TTCCAGTTACAATCGAGTTAACCGGAGCATTGATGTTGATTTTAAT
TGCTTGGATGTGCTGATCACACTGCAAACCTGGGTTGTGATATTAG
ACTTTTTTGGAATCGGCTCCACTGCAGACAACCACGCAATGAGGCT
GCCTCCTGAGGGCATTCTGCACAACGTGAAGTTGGAGCCACATGCC
TCCATGGAGTCTGGACTTCAGGATCCAGTGAACACCAAACTGGATC
TCAAGGTTCATTCACTTTCTCTAGTGCTGAATAAGACCACCAGTGA
GCTTGCCAAAGCAAATGTGTCCAAATTAGTAGCACACCTGGAAATG
ATTGAGGGAGACCTGGCCTTACAGGGCAGCATTGGGAGTCTGTCTC
TAAGTGACCTCACATGCCATGGAGAGTTCTACAGAGAACGGTTCAC
TACCAGTGGTGAAGAAGCACTCATCTTCCAGACTTTTAAATACGGA
CGGCCTGACCCTCTGCTCCGGAGAGAACACGACATTCGCGTGAGCC
TCCGGATGGCCTCTGTGCAGTATGTGCATACTCAGCGTTTCCAGGC
AGAGGTGGTGGCCTTCATTCAGCATTTCACTCAGCTGCAGGATGTC
TTAGGGCGCCAGCGAGCTGCTATTGAGGGGCAGACGGTGAGAGATC
AAGCCCAGCGCTGTTCACGGGTTCTCCTGGATATTGAGGCTGGTGC
TCCCGTTCTCTTGATCCCAGAAAGTTCCAGATCAAATAATCTGATT
GTAGCAAATTTGGGGAAGTTGAAAGTCAAAAATAAGTTTCTGTTTG
CTGGTTTTCCTGGCACCTTTTCCCTACAAGATAAGGAATCTGTGCC
TTCAGCTTCCCCAACGGGTATTCCCAAACACAGTCTGAGGAAAACG
ACAAGCACGGAGGAGCCCAGGGGAACCCATTCCCAGGGGCAGTTCA
CGATGCCTCTTGCTGGAATGAGCCTAGGAAGCCTGAAGAGTGAGTT
TGTGCCCAGTACCTCCACCAAGCAGCAAGGGCCGCAACCCACACTG
TCTGTTGGCCAAGAGTCCAGTAGTCCAGAAGACCATGTCTGCCTGC
TGGATTGCGTTGTCGTGGATCTCCAGGACATGGACATCTTTGCTGC
AGAGAGACATCCGAGAGAATACTCGAAGGCACCAGAGGATAGTAGT
GGAGATCTGATCTTCCCTTCCTATTTTGTGCGACAGACAGGAGGAA
GCCTCTTAACCGAGCCTTGTAGGCTGAAATTGCAGGTGGAAAGGAA
TTTGGACAAAGAAATAAGTCATACTGTGCCAGACATATCTATCCAT
GGCAATCTCTCCTCAGTCCACTGCTCTCTGGATCTGTATAAATACA
AGCTGATCCGCGGCTTATTAGAGAACAACCTGGGAGAACCCATAGA
GGAATTTATGCGGCCTTATGATTTACAAGATCCAAGAATTCATACT
GTCCTGAGTGGAGAAGTGTACACCTGTATGTGCTTCCTCATTGATA
TGGTGAATGTAAGTCTGGAGCTTAAAGATCCAAAAAGAAAAGAAGG
TGCTGGGTCCCTAGCCAGATTTGACTTCAAGAAATGCAAACTGCTC
TATGAAAGTTTTTCCAACCAAACCAAGTCCATTAACTTGGTTTCCC
ATTCCATGATGGCTTTTGACACCCGTTATGCTGGGCAGAAGACCAG
CCCTGGCATGACGAATGTGTTCAGCTGTATCTTTCAGCCCGCTAAG
AACAGCAGCACCACCCAAGGGTCCATTCAGATTGAACTACATTTCA
GATCTACCAAGGATTCCTCCTGCTTTACAGTAGTTCTCAACAATCT
CCGTGTGTTTCTCATATTTGACTGGCTACTGTTAGTCCATGATTTT
CTCCACACTCCCAGTGATATTAAGAAACAAAATCATGTTACTCCTT
CTCGCCACCGTAACTCTAGCAGCGAATCTGCTATAGTTCCCAAAAC
TGTGAAGAGTGGAGTAGTTACCAAGCGGTCTTCCCTTCCTGTGTCC
AATGAAAGGCACCTGGAGGTCAAGGTCAATGTAACAGGTACGGAGT
TTGTGGTCATTGAAGATGTGTCCTGCTTCGACACCAATGCCATTAT
TCTGAAAGGCACCACAGTGCTCACCTATAAGCCCCGGTTTGTTGAT
CGCCCCTTTTCAGGAAGTTTGTTTGGCATTGAGGTGTTTTCATGCC
GACTAGGGAATGAGCATGATACAGCTCTTTCAATTGTGGATCCCGT
ACAAATTCAAATGGAGTTGGTGGGGAATTCTTCTTATCAAAATAGT
TCAGGATTGATGGATGCATTCAATAGTGAAGATTTCCCACCTGTCC
TGGAGATTCAGTTACAAGCCCTGGATATCAGACTCTCCTATAATGA
TGTTCAGCTGTTTCTTGCCATTGCAAAATCCATCCCAGAGCAAGCT
AATGCTGCAGTGCCAGACTCAGTGGCCCTGGAGTCAGACTCCGTTG
GCACTTACCTTCCAGGTGCATCTCGCGTTGGAGAGGAAATCAGAGA
AGGGACAAGACACACCTTAGATCCTGTCTTGGAGTTACAGCTGGCT
AGGCTGCAGGAGCTGGGATTCAGCATGGATGATTGTCGCAAAGCTC
TTTTGGCGTGTCAAGGCCAATTGAAAAAGGCAGCAAGTTGGTTGTT
TAAGAATGCGGAACCTCTGAAGTCTCTTTCCTTGGCCTCCACCAGC
CGAGATAGCCCAGGGGCTGTGGCAGCGCCATTGATCTCTGGCGTGG
AGATCAAAGCTGAGAGTGTGTGCATCTGTTTCATCGATGACTGCAT
GGATTGTGATGTTCCTCTCGCTGAACTCACCTTTTCCCGTCTGAAT
TTTCTTCAGCGTGTAAGAACTAGCCCTGAAGGCTATGCCCACTTCA
CCCTTTCTGGAGATTATTATAACCGTGCTCTTTCAGGCTGGGAGCC
ATTTATTGAGCCTTGGCCATGCTCTGTATCCTGGCAACAGCAGGCA
GCTAGTCGTCTCCATCCTCCTCGACTGAAGCTAGAAGCCAAGGCCA
AACCTCGTTTGGATATCAATATCACTTCTGTGCTAATTGACCAGTA
TGTAAGTACCAAGGAATCGTGGATGGCAGATTACTGTAAAGATGAC
AAGGACATAGAGTCAGCTAAATCAGAAGACTGGATGGGCTCTTCGG
TGGATCCTCCATGTTTTGGACAAACAGAGGTGAAAACCCCCAAGCG
CCGGCAGCCATTTGTCCCCTTTGCTCTGAGGAACCACACGGGGTGC
ACTTTGTGGTTTGCCACCCTGACCACCACACCCACCAGAGCTGCAC
TCTCTCACAGTGGGAGTCCAGGGGTAGTTCCAGAAGGGAACGGAAC
ATTTCTCGATGATACTCACAATGTTAGTGAATGGCGAGAAGTCCTT
ACAGGTGAAGAGATTCCCTTTGAATTTGAAGCAAGAGGAAAGTTAA
GACACAGACACACCCATGACCTCCGGATTCATCAACTGCAAGTGAG
AGTAAATGGCTGGGAGCAAGTGAGCCCAGTGTCTGTGGACAAAGTC
GGGACCTTTTTTCGATATGCAGCACCAGATAAAAATTCATCTTCCT
CTACGATTGGCAGCCCAAGCAGCAGAACAAATATTATACATCCCCA
GGTTTATTTCTCTTCACTCCCACCAGTGCGGGTGGTCTTTGCAGTG
ACTATGGAAGGCAGTGCACGGAAAGTCATCACTGTCCGGTCAGCCC
TCATTGTGAGGAACAGACTTGAGACACCAATGGAACTAAGACTGGA
TAGCCCATCAGCTCCAGACAAGCCAGTGGTGCTTCCTGCTATCATG
CCAGGGGATTCGTTTGCTGTGCCTTTACACCTCACTTCTTGGCGGC
TACAGGCCCGGCCCAAAGGATTGGGTGTATTTTTCTGTAAGGCTCC
CATTCATTGGACCAATGTAGTGAAGACTGCAGAAATTAGTAGCAGT
AAACGAGAGTGCCACTCTATGGACACAGAAAAAAGCCGATTTTTCA
GGTTTTGTGTGGCTATAAAGAAAGAGAATTATCCAGATTATATGCC
CTCAAACATATTTTCTGACAGTGCAAAACAGATTTTCAGACAGCCT
GGGCATACCATATATCTCCTGCCAACTGTGGTAATCTGCAACTTGC
TACCCTGTGAACTTGATTTTTATGTTAAAGGAATGCCAATTAATGG
GACGCTGAAACCTGGCAAGGAGGCAGCTCTCCATACAGCTGATACA
TCCCAGAACATTGAGCTGGGGGTATCACTGGAGAATTTCCCCCTCT
GTAAAGAATTGCTCATTCCACCTGGAACCCAAAACTATATGGTGAG
AATGCGACTCTATGACGTCAACCGTCGGCAGCTGAACCTCACCATC
CGGATTGTGTGTCGAGCAGAAGGATCCTTAAAGATCTTCATTTCTG
CTCCATATTGGCTGATTAACAAAACAGGGTTGCCACTGATCTTCAG
ACAGGACAATGCCAAGACAGATGCTGCAGGCCAGTTTGAGGAGCAT
GAGCTGGCCCGTAGCCTGAGTCCTCTCTTATTCTGCTATGCTGACA
AAGAGCAGCCAAACCTCTGCACGATGAGAATCGGAAGGGGGATTCA
TCCAGAAGGCATGCCGGGCTGGTGTCAGGGCTTCTCCCTGGATGGT
GGTAGTGGTGTCCGAGCTTTGAAAGTCATCCAGCAAGGAAACCGCC
CAGGGCTGATCTATAACATTGGTATTGATGTCAAGAAAGGCCGAGG
TCGATACATTGATACCTGCATGGTCATCTTTGCCCCCCGTTACCTG
TTAGATAATAAATCATCTCACAAGCTTGCATTTGCACAGAGGGAAT
TTGCCAGGGGACAGGGAACAGCCAATCCCGAAGGTTACATTTCCAC
CCTTCCTGGTTCCAGTGTGGTGTTCCACTGGCCTCGGAATGACTAT
GATCAGCTATTGTGTGTCAGACTGATGGACGTTCCCAATTGTATTT
GGTCTGGAGGCTTTGAAGTCAACAAGAATAATTCCTTCCATATCAA
CATGAGGGATACCTTGGGAAAATGCTTCTTCCTACGAGTGGAAATT
ACTCTCCGAGGAGCTACGTATAGGATCTCATTTAGTGACACAGATC
AGTTACCTCCTCCTTTCCGAATTGACAACTTTTCTAAGGTCCCGGT
TGTCTTTACTCAGCATGGCGTAGCTGAACCCAGGCTCCGGACTGAA
GTGAAGCCCATGACTTCATTGGATTATGCCTGGGACGAACCCACCT
TGCCACCTTTTATCACTCTGACTGTTAAAGGGGCAGGGTCCTCTGA
GATCAACTGCAACATGAATGATTICCAGGATAATCGGCAGCTTTAT
TATGAAAATTTCATTTACATTGCTGCTACATATACATTCTCTGGCT
TGCAGGAGGGAACAGGCAGGCCTGTGGCTTCCAACAAGGCCATTAC
CTGTGCGGAGCTCGTTTTGGATGTCTCACCCAAGACACAAAGAGTC
ATTTTAAAAAAGAAGGAACCAGGAAAGCGTTCTCAGCTGTGGAGGA
TGACAGGAACAGGAATGCTGGCCCATGAGGGCTCCTCAGTTCCTCA
CAATCCCAATAAGCCCTCAGCCGCCCGCTCCACCGAGGGGTCTGCC
ATCTTAGATATTGCTGGTCTCGCTGCAGTGACTGACAACAGATACG
AGCCACTGATGCTGAGAAAGCCTGACCGCAGGCGAAGCACAACTCA
GACGTGGAGTTTCCGAGAAGGAAAACTGACCTGTGGGTTACATGGG
TTGGTCGTCCAGGCCAAAGGAGGACTTTCTGGTTTGTTTGATGGAG
CTGAAGTTGTTCTTGGTCCTGACACTTCCATGGAGCTTTTGGGGCC
AGTTCCACCTGAACAACAATTTATTAATCAAAAAATGAGACCTGGT
TCTGGAATGTTATCCATCAGAGTCATCCCAGATGGACCAACTAGAG
CACTCCAGATAACAGATTTCTGCCACCGGAAAAGCAGCCGTTCATA
TGAAGTGGATGAACTTCCTGTCACCGAACAAGAGCTGCAGAAATTA
AAGAATCCAGATACAGAGCAGGAATTGGAAGTGCTTGTGAGGTTAG
AAGGTGGAATTGGGTTGTCCTTAATTAATAAAGTCCCAGAAGAACT
GGTCTTTGCAAGTCTTACAGGAATCAATGTGCACTATACACAGCTG
GCAACCAGTCACATGCTTGAACTCAGCATACAGGATGTACAGGTGG
ACAATCAGCTCATTGGTACCACGCAGCCCTTCATGCTCTATGTGAC
TCCCCTGAGCAATGAGAATGAGGTCATCGAGACCGGCCCAGCTGTG
CAAGTCAACGCAGTGAAGTTCCCCAGTAAGAGTGCACTGACCAACA
TCTACAAGCATCTGATGATCACAGCTCAGAGATTCACAGTGCAAAT
TGAGGAGAAACTGCTCCTCAAGCTGCTAAGTTTCTTTGGCTACGAT
CAAGCAGAATCAGAGGTGGAAAAATATGATGAAAACCTCCATGAAA
AGACAGCTGAGCAAGGTGGAACACCAATTCGATACTACTTTGAAAA
TCTCAAAATCAGCATTCCTCAGATCAAGCTAAGTGTGTTCACCTCC
AACAAGCTCCCATTGGATCTTAAGGCCCTAAAAAGCACCTTGGGGT
TTCCTTTGATACGGTTTGAAGACGCTGTGATTAATCTAGATCCATT
CACTCGGGTACATCCCTATGAGACCAAGGAGTTCATCATCAATGAT
ATCCTCAAACATTTCCAGGAGGAACTCCTCAGCCAGGCAGCTCGAA
TCCTGGGATCAGTGGATTTTCTTGGCAATCCTATGGGGCTTTTGAA
TGATGTTTCTGAAGGGGTTACTGGACTGATAAAATATGGAAATGTC
GGGGGCCTCATCAGAAATGTTACACACGGAGTATCAAACTCTGCTG
CCAAGTTTGCTGGAACATTATCAGATGGCTTAGGGAAGACGATGGA
CAATCGGCATCAGTCAGAGCGGGAGTACATCAGGTACCATGCAGCC
ACAAGTGGTGAACACCTTGTAGCCGGCATCCATGGCCTGGCTCATG
GTATCATTGGTGGACTGACCAGTGTTATAACTTCGACAGTGGAAGG
TGTGAAAACAGAAGGGGGTGTCAGCGGTTTCATATCTGGCCTTGGA
AAAGGGCTTGTTGGCACTGTAACCAAGCCAGTGGCAGGCGCCCTGG
ATTTTGCATCAGAAACAGCCCAGGCGGTGAGAGACACAGCCACACT
CAGCGGCCCCAGGACTCAAGCACAGAGGGTTCGGAAACCGCGTTGC
TGCACGGGGCCCCAGGGGCTGCTTCCCCGATATTCTGAGAGCCAGG
CGGAAGGACAGGAGCAGCTCTTCAAACTCACAGACAACATACAGGA
CGAATTCTTCATCGCTGTGGAGAACATTGACAGCTACTGCGTGCTC
ATCTCCTCCAAAGCTGTTTACTTCCTGAAAAGTGGAGACTACGTGG
ATCGAGAAGCCATTTTCCTAGAAGTCAAATACGATGACCTCTACCA
CTGCCTTGTCTCCAAAGACCATGGGAAGGTGTATGTGCAGGTGACC
AAGAAAGCCGTGAGCACGAGCAGTGGAGTGTCCATCCCCGGCCCCT
CCCACCAGAAGCCCATGGTCCATGTGAAATCTGAGGTCCTTGCTGT
CAAGTTGTCACAAGAAATAAACTACGCAAAGAGCCTCTACTATGAA
CAGCAGCTTATGTTAAGACTCAGCGAAAACCGAGAGCAGCTGGAGC
TGGACTCCTGAAGCCCCGCTGCTGAGATGGGCGCTCCCGACACAGC
GCAGACCCACCAGGAGGAAAGAGGCCCAGCTCTCAGCTGACGATGG
AGGCAGAACCGGAGTCGGGTTTGGGGAAGTTGTCAAGGAATGAGGG
AAAGTAAATCCTCATGAGGAAAAGTACAAATGGAAATCGTATTAAT
TTGTGAGGCAGGGAGTTATTTTAGATTATGGGAAATAATTTTTAAA
GGTATTGGTTAAATAACGTTTAAAAACATGTACTGAGATGAATCTA
ATTTTTAGATTGCCCTGTATTTTGTTAACATGTATATATGTACAAC
AGTGTGTTTGTAAATATATAGGAACGTTTCTGAACAGGGTCTGTGC
TATGTGTAAAGGTTTGTTAACTGTAAAGTAATATAAAGTTATATTG
GATCTTCTATTGCACTAATTCTAGATGTCTAATTCAGGATACTGTC
TATAGAAAGGCATTCTTAAAAGTTAAAGAATGTTACGTCTTAGTTT
TGGAGACTAAAGTATTCCCAGTAAAGTGGGTTGAGGTGAGGGCTGT
GGTCCTGAAAGGGACGCCTTTGACATCGTGGCTGTCCAGTTGGGCT
GTGAGCTGTGGCACCCAGGACTGGCGCTGGCCCTTCAGAAGGATCT
AGGAGAGGGGCTTGGGAGCCCACTTTTAATTTCTCACCCCCATTTT
ACAAAGAGTGCTTAGATTCTTACAAATTATGATGTAAGTTATCCAT
TTGGCTTTTTCCTAACTAGTCTTACCAAACTTAGGGGGAAACCTGT
GCTCCATTACCACATGGGTGCAAGTCAGCATTGTAAGTTTTCTCAG
GTTATTATTATTAGAGAGGTTGGAAACATTGGTAAACTCTGTTGAT
TGAGAAGGAAAAAAAAAGTCCCATTGAACTGTTGCAACAAATCAGA
AATCCACATAAAAGTGCTCTCCTGCCTGGGCAGCAACAACCAAGAA
CAAAGCCCCGGGACTGTTTTCTTTTTAATAAAGCCACAGGCAGGCA
TCGTAGCTCCACAGCCCGAGGGGACACAGGATGGAAACCCCAGGAT
GAGAAGGGAGCAGGGAGAGTTCCAGAAAGGGGGATGAAATAGGAGT
ATTAAAAAGCTGCGTTGGTAAGTTTTTCATGGAACCAAGATTTGAC
AAAGGCATCTCTTATCCTTGGTTTTAAATTCCTGCTGGGAGCAAGG
CCTGGTATGAGCGCCCTGGGTCTTGTTTTTGGTGTTTCGCTTTTCT
GTAAGGATTAAGCAGATAGGGAGAAGGGAAAAGGGGCCTCACTTTA
GAATGAATGAGTCACCTTGTGATTTTTAAATTTTTATTTTAATAAA
GCTAATCAATTTCTACAACCTTGTCACATGTAGCTGAGTCTGGGAT
GACTCAGTGGATCAGTGGATGCGTGGAAGGTTTTGGTGTTTATAAC
TCATGACCCAAATCCTTCCAAGACACAAATGAAGCTAATCTTCTTC
CCCTCCCTTTCCTCTCCCACTTCCCCTCTGTGTCTGTGTCTGTGTG
TGTGTGTGTGTGTGTGTGTGTGTGCGCGCGCGCGCGTGCATGCAGA
GAGGAAGGAAGGGAGCTTTAGCACAAGTAGCTACATAGTGTTCAAG
GATCAGCTTTCTTGAACTCCGAGGCTTGGTTGCCTTTGCTGAACTC
CAAGTTAGTGGATTGCAGAATGGAAACTTGGCTTTTGCGGCACTGG
GTGAGTTTTAGTTTGTGTGTGTCTTGCTGGGGGGTGGTGATGATTG
TCTCAGCACTCACGCACTGCACAAGATGGCAGCAGGATACAGCACT
GCACAAGATGGCAGCTCCTCTGCAGCTTCCTCCTCAGCCTCCCTCC
TTGCACCCCCACAGGTTTGGCTTGTGGTTTTTGTCATCAGTAACCT
ACTGCCTGAGATCATGATCTCTTAAAAGATGAGACTCTCGGAAGGG
TTGATTGTATGCGTCAGTGAGCCTTCTATCACCTTCTGGAACAAAG
TCACTTGAAATCTCTTGATGAGATTAAGGAGTTTAGTGTTACTAAG
AAAATCTGCTTTGGGCCGCAGCAGTGCTGGGTGTTCTCAGACCTGA
CTGAGGAAGTTAGCTGCGGGCTGCCCTGTGGGCTGGTGCTTCAGGA
GGAATCCAGAGAAGTGTTCAGATGCCCCCCTTGGGCTCCTTTCTAA
TTTTAATCAGCTCTTTAAATAGCTGCCCATCTCCTGTGATTGCACA
ACCAAGCACTTTGACATTTGCACCTTAGGAGAGGCAGATGTTAAAA
TGGAATCCAAAGACCACCTAGGGCGGGGCTGGGTGGGAGATGGGAG
GGCCAACTGCGAGCTGCTCCACTTCTCAGCTCTCCCCTGCCCTGCA
GCCCTGGGCCAGACAAGGCCAGAAGGTTTCAGGGGCATTTGACATC
CCCTCCTGGTTCTCACCAGGAAAACATCCAAAGCTTTGGAGGAAAC
AGGCCCTGCCCCTGGCTCCTTAAATGCCCCGTCTCTTTGTAAACTG
ATATTCAGCCAGCAATGCCTAAGACTTTGTTAAGATCATTTCTACT
GCTTTTCTTTCTGCTTCAAACACACAGTTCGTCTCTGAGGAAAGTA
AAATAAATGGAATAAGAGTAAATTGGGTAAGGAGATATCCAAAGCT
ACCCAGTCCCTTGACCCAGCACAGTTGGCCGACCCGTGTCACTCCC
TGGCTGTCGCTGCTTCTCTGTGCTCACTGAAGGGTGAGCCAGGCCA
GTGCTTCCCCAGCCCCTGGGCCTGGTCACTACACAGTGGAAAACAG
ACAAGCGGCCCCTTCCCCAAATCCCAAGAGTGTCTTGCTGCTTGGT
GGGTGCTCATCGCAATGTTCTGAAGGCTCCAGGGCCACTTTGTTTG
TAAGTATGATCTGGGCCTCAAAATACCATAGTAGCTGCTTGATAAA
ATTCTAAAAATATCTGGTTCTCTATTATGTAAACACTATTACAGTC
ACCAGTGTGTGAAGACTCTTGAGTCTGGTTCTCATATCAGAGTCAT
CATTTTTCTTCCTGTGGAATAAAATGCCTTGTGGACTTCCCAAA
NCBI Reference Sequence Accession Number
NP_056193.2
(SEQ ID NO: 3)
MLEGLVAWVLNTYLGKYVNNLNTDQLSVALLKGAVELENLPLKKDA
LKELELPFEVKAGFIGKVTLQIPFYRPHVDPWVISISSLHLIGAPE
KIQDFNDEKEKLLERERKKALLQALEEKWKNDRQQKGESYWYSVTA
SVVTRIVENIELKIQDVHLRFEDGVTNPSHPFAFGICIKNVSMQNA
VNEPVQKLMRKKQLDVAEFSIYWDVDCTLLGDLPQMELQEAMARSM
ESRSHHYVLEPVFASALLKRNCSKKPLRSRHSPRIDCDIQLETIPL
KLSQLQYRQIMEFLKELERKERQVKFRRWKPKVAISKNCREWWYFA
LNANLYEIREQRKRCTWDFMLHRARDAVSYTDKYFNKLKGGLLSTD
DKEEMCRIEEEQSFEELKILRELVHDRFHKQEELAESLREPQFDSP
GACPGAPEPGGGSGMLQYLQSWFPGWGGWYGQQTPEGNVVEGLSAE
QQEQWIPEEILGTEEFFDPTADASCMNTYTKRDHVFAKLNLQLQRG
TVTLLHKEQGTPQMNESAFMQLEFSDVKLLAESLPRRNSSLLSVRL
GGLFLRDLATEGTMFPLLVFPNPQKEVGRVSQSFGLQTTSADRSDH
YPAADPDGPVFEMLYERNPAHSHFERRLNVSTRPLNILYNPQAIKK
VADFFYKGKVHTSGFGYQSELELRVAEAARRQYNKLKMQTKAEIRQ
TLDRLLVGDFIEESKRWTVRLDISAPQVIFPDDFKFKNPVLVVVDL
GRMLLTNTQDNSRRKSRDGSASEETQFSDDEYKTPLATPPNTPPPE
SSSSNGEKTPPFSGVEFSEEQLQAHLMSTKMYERYSLSFMDLQIMV
GRVKDNWKHVQDIDVGPTHVVEKFNVHLQLERRLIYTSDPKYPGAV
LSGNLPDLKIHINEDKISALKNCFALLTTPEMKTSDTQIKEKIFPQ
EEQRGSLQDSVMNLTQSIVLLEQHTREVLVESQLLLAEFKVNCMQL
GVESNGRYISVLKVFGTNAHFVKRPYDAEVSLTVHGLLLVDTMQTY
GADFDLLMASHKNLSFDIPTGSLRDSRAQSPVSGPNVAHLTDGATL
NDRSATSVSLDKILTKEQESLIKLEYQFVSSECPSMNLDSTLQVIS
LQVNNLDIILNPETIVELIGFLQKSFPKEKDDLSPQPLMTDFERSF
REQGTYQSTYEQNTEVAVEIHRLNLLLLRTVGMANREKYGRKIATA
SIGGTKVNVSMGSTFDMNGSLGCLQLMDLTQDNVKNQYVVSIGNSV
GYENIISDIGYFESVFVRMEDAALTEALSFTFVERSKQECFLNLKM
ASLHYNHSAKFLKELTLSMDELEENFRGMLKSAATKVTTVLATKTA
EYSEMVSLFETPRKTREPFILEENEIYGFDLASSHLDTVKLILNIN
IESPVVSIPRKPGSPELLVGHLGQIFIQNFVAGDDESRSDRLQVEI
KDIKLYSLNCTQLAGREAVGSEGSRMFCPPSGSGSANSQEEAHFTR
HDFFESLHRGQAFHILNNTTIQFKLEKIPIERESELTFSLSPDDLG
TSSIMKIEGKFVNPVQVVLAKHVYEQVLQTLDNLVYSEDLNKYPAS
ATSSPCPDSPLPPLSTCGESSVERKENGLFSHSSLSNTSQKSLSVK
EVKSFTQIQATFCISELQVQLSGDLTLGAQGLVSLKFQDFEVEFSK
DHPQTLSIQIALHSLLMEDLLEKNPDSKYKNLMVSRGAPKPSSLAQ
KEYLSQSCPSVSNVEYPDMPRSLPSHMEEAPNVFQLYQRPTSASRK
KQKEVQDKDYPLTPPPSPTVDEPKILVGKSKFDDSLVHINIFLVDK
KHPEFSSSYNRVNRSIDVDFNCLDVLITLQTWVVILDFFGIGSTAD
NHAMRLPPEGILHNVKLEPHASMESGLQDPVNTKLDLKVHSLSLVL
NKTTSELAKANVSKLVAHLEMIEGDLALQGSIGSLSLSDLTCHGEF
YRERFTTSGEEALIFQTFKYGRPDPLLRREHDIRVSLRMASVQYVH
TQRFQAEVVAFIQHFTQLQDVLGRQRAAIEGQTVRDQAQRCSRVLL
DIEAGAPVLLIPESSRSNNLIVANLGKLKVKNKFLFAGFPGTFSLQ
DKESVPSASPTGIPKHSLRKTTSTEEPRGTHSQGQFTMPLAGMSLG
SLKSEFVPSTSTKQQGPQPTLSVGQESSSPEDHVCLLDCVVVDLQD
MDIFAAERHPREYSKAPEDSSGDLIFPSYFVRQTGGSLLTEPCRLK
LQVERNLDKEISHTVPDISIHGNLSSVHCSLDLYKYKLIRGLLENN
LGEPIEEFMRPYDLQDPRIHTVLSGEVYTCMCFLIDMVNVSLELKD
PKRKEGAGSLARFDFKKCKLLYESFSNQTKSINLVSHSMMAFDTRY
AGQKTSPGMTNVFSCIFQPAKNSSTTQGSIQIELHFRSTKDSSCFT
VVLNNLRVFLIFDWLLLVHDFLHTPSDIKKQNHVTPSRHRNSSSES
AIVPKTVKSGVVTKRSSLPVSNERHLEVKVNVTGTEFVVIEDVSCF
DTNAIILKGTTVLTYKPRFVDRPFSGSLFGIEVFSCRLGNEHDTAL
SIVDPVQIQMELVGNSSYQNSSGLMDAFNSEDFPPVLEIQLQALDI
RLSYNDVQLFLAIAKSIPEQANAAVPDSVALESDSVGTYLPGASRV
GEEIREGTRHTLDPVLELQLARLQELGFSMDDCRKALLACQGQLKK
AASWLFKNAEPLKSLSLASTSRDSPGAVAAPLISGVEIKAESVCIC
FIDDCMDCDVPLAELTFSRLNFLQRVRTSPEGYAHFTLSGDYYNRA
LSGWEPFIEPWPCSVSWQQQAASRLHPPRLKLEAKAKPRLDINITS
VLIDQYVSTKESWMADYCKDDKDIESAKSEDWMGSSVDPPCFGQSL
PLVYLRTRSTASLTNLEHQIYARAEVKTPKRRQPFVPFALRNHTGC
TLWFATLTTTPTRAALSHSGSPGVVPEGNGTFLDDTHNVSEWREVL
TGEEIPFEFEARGKLRHRHTHDLRIHQLQVRVNGWEQVSPVSVDKV
GTFFRYAAPDKNSSSSTIGSPSSRTNIIHPQVYFSSLPPVRVVFAV
TMEGSARKVITVRSALIVRNRLETPMELRLDSPSAPDKPVVLPAIM
PGDSFAVPLHLTSWRLQARPKGLGVFFCKAPIHWTNVVKTAEISSS
KRECHSMDTEKSRFFRFCVAIKKENYPDYMPSNIFSDSAKQIFRQP
GHTIYLLPTVVICNLLPCELDFYVKGMPINGTLKPGKEAALHTADT
SQNIELGVSLENFPLCKELLIPPGTQNYMVRMRLYDVNRRQLNLTI
RIVCRAEGSLKIFISAPYWLINKTGLPLIFRQDNAKTDAAGQFEEH
ELARSLSPLLFCYADKEQPNLCTMRIGRGIHPEGMPGWCQGFSLDG
GSGVRALKVIQQGNRPGLIYNIGIDVKKGRGRYIDTCMVIFAPRYL
LDNKSSHKLAFAQREFARGQGTANPEGYISTLPGSSVVFHWPRNDY
DQLLCVRLMDVPNCIWSGGFEVNKNNSFHINMRDTLGKCFFLRVEI
TLRGATYRISFSDTDQLPPPFRIDNFSKVPVVFTQHGVAEPRLRTE
VKPMTSLDYAWDEPTLPPFITLTVKGAGSSEINCNMNDFQDNRQLY
YENFIYIAATYTFSGLQEGTGRPVASNKAITCAELVLDVSPKTQRV
ILKKKEPGKRSQLWRMTGTGMLAHEGSSVPHNPNKPSAARSTEGSA
ILDIAGLAAVTDNRYEPLMLRKPDRRRSTTQTWSFREGKLTCGLHG
LVVQAKGGLSGLFDGAEVVLGPDTSMELLGPVPPEQQFINQKMRPG
SGMLSIRVIPDGPTRALQITDFCHRKSSRSYEVDELPVTEQELQKL
KNPDTEQELEVLVRLEGGIGLSLINKVPEELVFASLTGINVHYTQL
ATSHMLELSIQDVQVDNQLIGTTQPFMLYVTPLSNENEVIETGPAV
QVNAVKFPSKSALTNIYKHLMITAQRFTVQIEEKLLLKLLSFFGYD
QAESEVEKYDENLHEKTAEQGGTPIRYYFENLKISIPQIKLSVFTS
NKLPLDLKALKSTLGFPLIRFEDAVINLDPFTRVHPYETKEFIIND
ILKHFQEELLSQAARILGSVDFLGNPMGLLNDVSEGVTGLIKYGNV
GGLIRNVTHGVSNSAAKFAGTLSDGLGKTMDNRHQSEREYIRYHAA
TSGEHLVAGIHGLAHGIIGGLTSVITSTVEGVKTEGGVSGFISGLG
KGLVGTVTKPVAGALDFASETAQAVRDTATLSGPRTQAQRVRKPRC
CTGPQGLLPRYSESQAEGQEQLFKLTDNIQDEFFIAVENIDSYCVL
ISSKAVYFLKSGDYVDREAIFLEVKYDDLYHCLVSKDHGKVYVQVT
KKAVSTSSGVSIPGPSHQKPMVHVKSEVLAVKLSQEINYAKSLYYE
QQLMLRLSENREQLELDS
NCBI Reference Sequence Accession Number
NP_060626.2
(SEQ ID NO: 4)
MLEGLVAWVLNTYLGKYVNNLNTDQLSVALLKGAVELENLPLKKDA
LKELELPFEVKEKWKNDRQQKGESYWYSVTASVVTRIVENIELKIQ
DVHLRFEDGVTNPSHPFAFGICIKNVSMQNAVNEPVQKLMRKKQLD
VAEFSIYWDVDCTLLGDLPQMELQEAMARSMESRSHHYVLEPVFAS
ALLKRNCSKKPLRSRHSPRIDCDIQLETIPLKLSQLQYRQIMEFLK
ELERKERQVKFRRWKPKVAISKNCREWWYFALNANLYEIREQRKRC
TWDFMLHRARDAVSYTDKYFNKLKGGLLSTDDKEEMCRIEEEQSFE
ELKILRELVHDRFHKQEELAESLREPQFDSPGACPGAPEPGGGSGM
LQYLQSWFPGWGGWYGQQTPEGNVVEGLSAEQQEQWIPEEILGTEE
FFDPTADASCMNTYTKRDHVFAKLNLQLQRGTVTLLHKEQGTPQMN
ESAFMQLEFSDVKLLAESLPRRNSSLLSVRLGGLFLRDLATEGTMF
PLLVFPNPQKEVGRVSQSFGLQTTSADRSDHYPAADPDGPVFEMLY
ERNPAHSHFERRLNVSTRPLNIIYNPQAIKKVADFFYKGKVHTSGF
GYQSELELRVAEAARRQYNKLKMQTKAEIRQTLDRLLVGDFIEESK
RWTVRLDISAPQVIFPDDFKFKNPVLVVVDLGRMLLTNTQDNSRRK
SRDGSASEETQFSDDEYKTPLATPPNTPPPESSSSNGEKTPPFSGV
EFSEEQLQAHLMSTKMYERYSLSFMDLQIMVGRVKDNWKHVQDIDV
GPTHVVEKFNVHLQLERRLIYTSDPKYPGAVLSGNLPDLKIHINED
KISALKNCFALLTTPEMKTSDTQIKEKIFPQEEQRGSLQDSVMNLT
QSIVLLEQHTREVLVESQLLLAEFKVNCMQLGVESNGRYISVLKVF
GTNAHFVKRPYDAEVSLTVHGLLLVDTMQTYGADFDLLMASHKNLS
FDIPTGSLRDSRAQSPVSGPNVAHLTDGATLNDRSATSVSLDKILT
KEQESLIKLEYQFVSSECPSMNLDSTLQVISLQVNNLDIILNPETI
VELIGFLQKSFPKEKDDLSPQPLMTDFERSFREQGTYQSTYEQNTE
VAVEIHRLNLLLLRTVGMANREKYGRKIATASIGGTKVNVSMGSTF
DMNGSLGCLQLMDLTQDNVKNQYVVSIGNSVGYENIISDIGYFESV
FVRMEDAALTEALSFTFVERSKQECFLNLKMASLHYNHSAKFLKEL
TLSMDELEENFRGMLKSAATKVTTVLATKTAEYSEMVSLFETPRKT
REPFILEENEIYGFDLASSHLDTVKLILNINIESPVVSIPRKPGSP
ELLVGHLGQIFIQNFVAGDDESRSDRLQVEIKDIKLYSLNCTQLAG
REAVGSEGSRMFCPPSGSGSANSQEEAHFTRHDFFESLHRGQAFHI
LNNTTIQFKLEKIPIERESELTFSLSPDDLGTSSIMKIEGKFVNPV
QVVLAKHVYEQVLQTLDNLVYSEDLNKYPASATSSPCPDSPLPPLS
TCGESSVERKENGLFSHSSLSNTSQKSLSVKEVKSFTQIQATFCIS
ELQVQLSGDLTLGAQGLVSLKFQDFEVEFSKDHPQTLSIQIALHSL
LMEDLLEKNPDSKYKNLMVSRGAPKPSSLAQKEYLSQSCPSVSNVE
YPDMPRSLPSHMEEAPNVFQLYQRPTSASRKKQKEVQDKDYPLTPP
PSPTVDEPKILVGKSKFDDSLVHINIFLVDKKHPEFSSSYNRVNRS
IDVDFNCLDVLITLQTWVVILDFFGIGSTADNHAMRLPPEGILHNV
KLEPHASMESGLQDPVNTKLDLKVHSLSLVLNKTTSELAKANVSKL
VAHLEMIEGDLALQGSIGSLSLSDLTCHGEFYRERFTTSGEEALIF
QTFKYGRPDPLLRREHDIRVSLRMASVQYVHTQRFQAEVVAFIQHF
TQLQDVLGRQRAAIEGQTVRDQAQRCSRVLLDIEAGAPVLLIPESS
RSNNLIVANLGKLKVKNKFLFAGFPGTFSLQDKESVPSASPTGIPK
HSLRKTTSTEEPRGTHSQGQFTMPLAGMSLGSLKSEFVPSTSTKQQ
GPQPTLSVGQESSSPEDHVCLLDCVVVDLQDMDIFAAERHPREYSK
APEDSSGDLIFPSYFVRQTGGSLLTEPCRLKLQVERNLDKEISHTV
PDISIHGNLSSVHCSLDLYKYKLIRGLLENNLGEPIEEFMRPYDLQ
DPRIHTVLSGEVYTCMCFLIDMVNVSLELKDPKRKEGAGSLARFDF
KKCKLLYESFSNQTKSINLVSHSMMAFDTRYAGQKTSPGMTNVFSC
IFQPAKNSSTTQGSIQIELHFRSTKDSSCFTVVLNNLRVFLIFDWL
LLVHDFLHTPSDIKKQNHVTPSRHRNSSSESAIVPKTVKSGVVTKR
SSLPVSNERHLEVKVNVTGTEFVVIEDVSCFDTNAIILKGTTVLTY
KPRFVDRPFSGSLFGIEVFSCRLGNEHDTALSIVDPVQIQMELVGN
SSYQNSSGLMDAFNSEDFPPVLEIQLQALDIRLSYNDVQLFLAIAK
SIPEQANAAVPDSVALESDSVGTYLPGASRVGEEIREGTRHTLDPV
LELQLARLQELGFSMDDCRKALLACQGQLKKAASWLFKNAEPLKSL
SLASTSRDSPGAVAAPLISGVEIKAESVCICFIDDCMDCDVPLAEL
TFSRLNFLQRVRTSPEGYAHFTLSGDYYNRALSGWEPFIEPWPCSV
SWQQQAASRLHPPRLKLEAKAKPRLDINITSVLIDQYVSTKESWMA
DYCKDDKDIESAKSEDWMGSSVDPPCFGQTEVKTPKRRQPFVPFAL
RNHTGCTLWFATLTTTPTRAALSHSGSPGVVPEGNGTFLDDTHNVS
EWREVLTGEEIPFEFEARGKLRHRHTHDLRIHQLQVRVNGWEQVSP
VSVDKVGTFFRYAAPDKNSSSSTIGSPSSRTNIIHPQVYFSSLPPV
RVVFAVTMEGSARKVITVRSALIVRNRLETPMELRLDSPSAPDKPV
VLPAIMPGDSFAVPLHLTSWRLQARPKGLGVFFCKAPIHWTNVVKT
AEISSSKRECHSMDTEKSRFFRFCVAIKKENYPDYMPSNIFSDSAK
QIFRQPGHTIYLLPTVVICNLLPCELDFYVKGMPINGTLKPGKEAA
LHTADTSQNIELGVSLENFPLCKELLIPPGTQNYMVRMRLYDVNRR
QLNLTIRIVCRAEGSLKIFISAPYWLINKTGLPLIFRQDNAKTDAA
GQFEEHELARSLSPLLFCYADKEQPNLCTMRIGRGIHPEGMPGWCQ
GFSLDGGSGVRALKVIQQGNRPGLIYNIGIDVKKGRGRYIDTCMVI
FAPRYLLDNKSSHKLAFAQREFARGQGTANPEGYISTLPGSSVVFH
WPRNDYDQLLCVRLMDVPNCIWSGGFEVNKNNSFHINMRDTLGKCF
FLRVEITLRGATYRISFSDTDQLPPPFRIDNFSKVPVVFTQHGVAE
PRLRTEVKPMTSLDYAWDEPTLPPFITLTVKGAGSSEINCNMNDFQ
DNRQLYYENFIYIAATYTFSGLQEGTGRPVASNKAITCAELVLDVS
PKTQRVILKKKEPGKRSQLWRMTGTGMLAHEGSSVPHNPNKPSAAR
STEGSAILDIAGLAAVTDNRYEPLMLRKPDRRRSTTQTWSFREGKL
TCGLHGLVVQAKGGLSGLFDGAEVVLGPDTSMELLGPVPPEQQFIN
QKMRPGSGMLSIRVIPDGPTRALQITDFCHRKSSRSYEVDELPVTE
QELQKLKNPDTEQELEVLVRLEGGIGLSLINKVPEELVFASLTGIN
VHYTQLATSHMLELSIQDVQVDNQLIGTTQPFMLYVTPLSNENEVI
ETGPAVQVNAVKFPSKSALTNIYKHLMITAQRFTVQIEEKLLLKLL
SFFGYDQAESEVEKYDENLHEKTAEQGGTPIRYYFENLKISIPQIK
LSVFTSNKLPLDLKALKSTLGFPLIRFEDAVINLDPFTRVHPYETK
EFIINDILKHFQEELLSQAARILGSVDFLGNPMGLLNDVSEGVTGL
IKYGNVGGLIRNVTHGVSNSAAKFAGTLSDGLGKTMDNRHOSEREY
IRYHAATSGEHLVAGIHGLAHGIIGGLTSVITSTVEGVKTEGGVSG
FISGLGKGLVGTVTKPVAGALDFASETAQAVRDTATLSGPRTQAQR
VRKPRCCTGPQGLLPRYSESQAEGQEQLFKLTDNIQDEFFIAVENI
DSYCVLISSKAVYFLKSGDYVDREAIFLEVKYDDLYHCLVSKDHGK
VYVQVTKKAVSTSSGVSIPGPSHQKPMVHVKSEVLAVKLSQEINYA
KSLYYEQQLMLRLSENREQLELDS
Mfn1 nucleic acid sequence NM_033540
(SEQ ID NO: 5)
ACTCCCCCTGCCTCCTCTCCGCCTTTAACTTCTCGGGAAGATGAGG
CAGTTTGGCATCTGTGGCCGAGTTGCTGTTGCCGGGTGATAGTTGG
AGCGGAGACTTAGCATAATGGCAGAACCTGTTTCTCCACTGAAGCA
CTTTGTGCTGGCTAAGAAGGCGATTACTGCAATCTTTGACCAGTTA
CTGGAGTTTGTTACTGAAGGATCACATTTTGTTGAAGCAACATATA
AGAATCCGGAACTTGATCGAATAGCCACTGAAGATGATCTGGTAGA
AATGCAAGGATATAAAGACAAGCTTTCCATCATTGGTGAGGTGCTA
TCTCGGAGACACATGAAGGTGGCATTTTTTGGCAGGACAAGCAGTG
GGAAGAGCTCTGTTATCAATGCAATGTTGTGGGATAAAGTTCTCCC
TAGTGGGATTGGCCATATAACCAATTGCTTCCTAAGTGTTGAAGGA
ACTGATGGAGATAAAGCCTATCTTATGACAGAAGGATCAGATGAAA
AAAAGAGTGTGAAGACAGTTAATCAACTGGCCCATGCCCTTCACAT
GGACAAAGATTTGAAAGCTGGCTGTCTTGTACGTGTGTTTTGGCCA
AAAGCAAAATGTGCCCTCTTGAGAGATGACCTGGTGTTAGTAGACA
GTCCAGGCACAGATGTCACTACAGAGCTGGATAGCTGGATTGATAA
GTTTTGCCTAGATGCTGATGTCTTTGTTTTGGTCGCAAACTCTGAA
TCAACACTAATGAATACGGAAAAACACTTTTTTCACAAGGTGAATG
AGCGGCTTTCCAAGCCTAATATTTTCATTCTCAATAATCGTTGGGA
TGCCTCTGCATCAGAGCCAGAATATATGGAAGACGTACGCAGACAG
CACATGGAAAGATGCCTGCATTTCTTGGTGGAGGAGCTCAAAGTTG
TAAATGCTTTAGAAGCACAGAATCGTATCTTCTTTGTTTCAGCAAA
GGAAGTTCTTAGTGCTAGAAAGCAAAAAGCACAGGGGATGCCAGAA
AGTGGTGTGGCACTTGCTGAAGGATTTCATGCAAGATTACAGGAAT
TTCAGAATTTTGAACAAATCTTTGAGGAGTGTATCTCGCAGTCAGC
AGTGAAAACAAAGTTCGAACAGCACACTATCAGAGCTAAACAGATA
CTAGCTACTGTGAAAAACATAATGGATTCAGTAAACCTGGCAGCTG
AAGATAAAAGGCATTATTCAGTGGAAGAGAGGGAAGACCAAATTGA
TAGACTGGACTTTATTCGAAACCAGATGAACCTTTTAACACTGGAT
GTTAAGAAAAAAATCAAGGAGGTTACCGAGGAGGTGGCAAACAAAG
TTTCATGTGCAATGACAGATGAAATTTGTCGACTGTCTGTTTTGGT
TGATGAATTTTGTTCAGAGTTTCATCCTAATCCAGATGTATTAAAA
ATATATAAAAGTGAATTAAATAAGCACATAGAGGATGGTATGGGAA
GAAATTTGGCTGATCGATGCACCGATGAAGTAAACGCCTTAGTGCT
TCAGACCCAGCAAGAAATTATTGAAAATTTGAAGCCATTACTTCCA
GCTGGTATACAGGATAAACTACATACACTGATCCCTTGCAAGAAAT
TTGATCTCAGTTATAATCTAAATTACCACAAGTTATGTTCAGATTT
TCAAGAGGATATTGTATTTCGTTTTTCCCTGGGCTGGTCTTCCCTT
GTACATCGATTTTTGGGCCCTAGAAATGCTCAAAGGGTGCTCCTAG
GATTATCAGAGCCTATCTTTCAGCTCCCTAGATCTTTAGCTTCTAC
TCCCACTGCTCCTACCACTCCAGCAACGCCAGATAATGCATCACAG
GAAGAACTCATGATTACATTAGTAACAGGATTGGCGTCCGTTACAT
CTAGAACTTCTATGGGCATCATTATTGTTGGAGGAGTGATTTGGAA
AACTATAGGCTGGAAACTCCTATCTGTTTCATTAACTATGTATGGA
GCTTTGTATCTTTATGAAAGACTGAGCTGGACCACCCATGCCAAGG
AGCGAGCCTTTAAACAGCAGTTTGTAAACTATGCAACTGAAAAACT
GAGGATGATTGTTAGCTCCACGAGTGCAAACTGCAGTCACCAAGTA
AAACAACAAATAGCTACCACTTTTGCTCGCCTGTGCCAACAAGTTG
ATATTACTCAAAAACAGCTGGAAGAAGAAATTGCTAGATTACCCAA
AGAAATAGATCAGTTGGAGAAAATACAAAACAATTCAAAGCTCTTA
AGAAATAAAGCTGTTCAACTTGAAAATGAGCTGGAGAATTTTACTA
AGCAGTTTCTACCTTCAAGCAATGAAGAATCCTAACAATAGAGATT
GCTTTGGTGACCATGATAGGAGGAAACGAAACTTGTAAGATTGGAA
CAGTTGTTATTTTTATGAAATTACTTTAAATATGAATTGTACTAAC
TGTACCTAAATAGCAAAGCCCTGTGTAGATTCTGGTAATGATCTGT
CTCAGGGTATGTGTATTTTTGAAGAGTGTTATGTCCTTAGTTTTAA
TTTTGAGTAAAGAAAAGGCTAAAATCATGAATTAGTTACAAGCAAC
AGTACCAACTTATGTGACCCCTGAGGGGTGGGGCTGTGAGCTCTTA
ATTTGTTTTTGATTCTGAAAAACTCTGCTTCCTGGCATCCAGGAGT
TAGAGATTGAGCCTTTCATCTTCTTTCTCAAAACTAGTTTTTGATG
CTTTCTTTCATGGGAATAGTCACTTTTTTATTTAGTAAATCGCATT
GCTGGAACCACCAAGGAGTGTGGAATGTCCTTGAGTGTATTATTTA
TGCAAGTCACAGTCACGTTGCCATCATGGCAGCTATGTGAAACACT
AATAAATGTGTTTTTACTTTTTATTCCCGTTAAAACTGATGTAAAA
CAGGATAAAGGCTTGTTATAGTCACTTATAAGTATCTGGGTCTAAG
TAATTTCCTTAGATGTTTCTAAAGAAACATTTTCAGCTTTGCTCCC
ATTATGATTCCAATAAGGAACGCTTTCCTAGTGCAATTTTAGGAGT
AAAGTTTGAAGAGATAAAAATAGCCAAAGATAGGAGACGTCTGAAT
TTTGAATGATAAACAGTGATGTTTTAAAAAAGCTGTTGTTCTTCAG
GAGGCATTTGCCTAGGATATTGCTGGATTATACCCCATTGGAGGCT
TTTAATTTTATTTGTATGAATTTTCCAGGATTTCATTAAAAATTAT
TATTGTATTTTTTACCTTAATGAAAGATTTTGGGTTCAAATATCTT
TCTATATTAAAAGCTGATTGAGTCTGTACATATGTAAATTATGCCT
AGTGGAGGTTCTGTTGACTTTCTTCCCCACTGTGGAAGAGGCCAGT
TTTGCCTCCATTTGCACATTCATTTCAGTTATTTCTGATCCATAAA
TATAACATTTACAAAATTCTTCCTTGAGCTGGTGGAAATGCCTCAC
CAGTTTCCTCTTTAATGAATCAAATAAAATCTTTAACTGATGTTAA
AAAAAATTGATTGAAACTCAGATGGAATGGAAATGTACAAAAATGA
CACCATTCTAGGAATTTGCTAGACAAAATGTAGGACTACCAGATCA
GTATCTCCTAGACACTTGTTAGAAATGCACAATCCCGGGGAACGCA
GTACATTTGGCCACATGTAGTTTATGTTTCCTTTTCATGGGAGGGA
TAAAATTTAAAGCTTTTTTTTTCTTTGAATACAGTCCTTCCTTTTC
TATGCCTTTTAAGGCTTCTAGATGCTATTCAGCCTTTTTACAGCAG
GTGCAACTCTATTTTTCAAGGTATCTTAGAAGATAACACTAGGCCA
TTGAAGCCTTTCAAAAATATATTTTTATGCAAATTGACACGAGTGC
AGTATACTAATGCAAATTAATTTTGGTGTTTAGCTTTTATTGCTCA
TTTATAACCCAAGAAATGGTACAGACCACAGTAAGTGGGATTAGGT
AACAACTACAACATGGAAAAACAGTGTCTAAGATCACGTACTGCTA
ATTTAGGTTACAAAACTCAAGGCTGTAACCTTTATATGAAAGTACT
TAAGCTTGAAAGTTCATAATAGTTTGGTATCACCTCATTAGTATAG
TACAGTGGTTCTCAAAGTTTGGTCCTGGGTCATCGACATTACTTCT
TTTTCTTTCTGAGACAGGGTCTCAGTCTGTCACCCAGGATGGAGTG
CAGTGGCGTGATCATGGCTCACTGCAGCCTCAACCTCTCGGGTTCA
GGTGATCCTCCCACATCAGCCTCCTGGGTGGCTGGGACTACAGGCA
CACGCCACCATGCCTGGCTAGTTTTTGTAAAGACGAGGTTTCCCCA
TGTTGTGCAGGCTGGTCTTGAACTCACAGACTCAAGTGATCCTTTT
ACCTCAGCCTCCCAAACTGTTGGGATAACAGGTGTAAGCCACCACA
CACAGCTATAATCAACCTTCAAACTTATAAAAAGTGTGGATCCTTG
GGTCTGAACCCAGACCTATGGAGTCAGACAGTAGGTTTGAGGCCCA
GCAATCTATGGTTTAACAAGCCATCCAGGTGTTTCTGATGCACAGT
GAAATTGGGGTACCACTGGTATTAGGTTTGGTATGGCAACTTTTTC
ATCACTTGTTTTATGTAGTTGTCTGATCAATTGTGAAAACATAATG
AATGTTGGAAATGGAACAGTAAAATAACGAAAGCCAACTTTTTTTT
TTTTTTTTTTTTTTTTTGAGACGGAGTCTCGCTCTGTCGCCCAGGC
TGGAGTGCAGTGGCGCGATCTCGGCTCACTGCAAGCTCCGCCTCCC
GGGTTCACGCCATTCTCCTGCCTCAGCCTCCCGAGTAGCTGGGACT
ACAGGCGCCCGCCACCACGCCCGGCTAATTTTTTGTATTTTTAGTA
GAGACGGGGTTTCACCGTGTTAGCCAGGATGGTCTCGATCTCCTGA
CCTCGTGATCCACCCGCCTCGGCCTCCCAAAGTGCTGGGATTACAG
GCGTGAGCCACCGCGCCCGGCCGAAAGCCAACTCTTATGCCTAGAA
ATATGTGCACCTATGACCAAGCCCATGAATTATACAGGAATTATGT
AATTATGAGTGATGTACTTCAAAGTTATTGCACATACACTTGTTTA
CTTTGTATGTTTGCAGGATTAAACTTTGTATAATCTTTTTACAAAA
ATTTTTTTTCAGTATGCAAGCTTGCAAGATGAAAATAAAACCTGTT
TGCCTGATAGTTGA
Mfn1 amino acid sequence NP_284941
(SEQ ID NO: 6)
MAEPVSPLKHFVLAKKAITAIFDQLLEFVTEGSHFVEATYKNPELD
RIATEDDLVEMQGYKDKLSIIGEVLSRRHMKVAFFGRTSSGKSSVI
NAMLWDKVLPSGIGHITNCFLSVEGTDGDKAYLMTEGSDEKKSVKT
VNQLAHALHMDKDLKAGCLVRVFWPKAKCALLRDDLVLVDSPGTDV
TTELDSWIDKFCLDADVFVLVANSESTLMNTEKHFFHKVNERLSKP
NIFILNNRWDASASEPEYMEDVRRQHMERCLHFLVEELKVVNALEA
QNRIFFVSAKEVLSARKQKAQGMPESGVALAEGFHARLQEFQNFEQ
IFEECISQSAVKTKFEQHTIRAKQILATVKNIMDSVNLAAEDKRHY
SVEEREDQIDRLDFIRNQMNLLTLDVKKKIKEVTEEVANKVSCAMT
DEICRLSVLVDEFCSEFHPNPDVLKIYKSELNKHIEDGMGRNLADR
CTDEVNALVLQTQQEIIENLKPLLPAGIQDKLHTLIPCKKFDLSYN
LNYHKLCSDFQEDIVFRFSLGWSSLVHRFLGPRNAQRVLLGLSEPI
FQLPRSLASTPTAPTTPATPDNASQEELMITLVTGLASVTSRTSMG
IIIVGGVIWKTIGWKLLSVSLTMYGALYLYERLSWTTHAKERAFKQ
QFVNYATEKLRMIVSSTSANCSHQVKQQIATTFARLCQQVDITQKQ
LEEEIARLPKEIDQLEKIQNNSKLLRNKAVQLENELENFTKQFLPS
SNEES
Mfn2 nucleic acid sequence NM_001127660
(SEQ ID NO: 7)
GTAGTGAGTGTGATGGCCGCCGCGAGGCCGGGAAGGTGAAGCGCAA
TGTCCCTGCTCTTCTCTCGATGCAACTCTATCGTCACAGTCAAGAA
AAATAAGAGACACATGGCTGAGGTGAATGCATCCCCACTTAAGCAC
TTTGTCACTGCCAAGAAGAAGATCAATGGCATTTTTGAGCAGCTGG
GGGCCTACATCCAGGAGAGCGCCACCTTCCTTGAAGACACGTACAG
GAATGCAGAACTGGACCCCGTTACCACAGAAGAACAGGTTCTGGAC
GTCAAAGGTTACCTATCCAAAGTGAGAGGCATCAGTGAGGTGCTGG
CTCGGAGGCACATGAAAGTGGCTTTTTTTGGCCGGACGAGCAATGG
GAAGAGCACCGTGATCAATGCCATGCTCTGGGACAAAGTTCTGCCC
TCTGGGATTGGCCACACCACCAATTGCTTCCTGCGGGTAGAGGGCA
CAGATGGCCATGAGGCCTTTCTCCTTACCGAGGGCTCAGAGGAAAA
GAGGAGTGCCAAGACTGTGAACCAGCTGGCCCATGCCCTCCACCAG
GACAAGCAGCTCCATGCCGGCAGCCTAGTGAGTGTGATGTGGCCCA
ACTCTAAGTGCCCACTTCTGAAGGATGACCTCGTTTTGATGGACAG
CCCTGGTATTGATGTCACCACAGAGCTGGACAGCTGGATTGACAAG
TTTTGTCTGGATGCTGATGTGTTTGTGCTGGTGGCCAACTCAGAGT
CCACCCTGATGCAGACGGAAAAGCACTTCTTCCACAAGGTGAGTGA
GCGTCTCTCCCGGCCAAACATCTTCATCCTGAACAACCGCTGGGAT
GCATCTGCCTCAGAGCCCGAGTACATGGAGGAGGTGCGGCGGCAGC
ACATGGAGCGTTGTACCAGCTTCCTGGTGGATGAGCTGGGCGTGGT
GGATCGATCCCAGGCCGGGGACCGCATCTTCTTTGTGTCTGCTAAG
GAGGTGCTCAACGCCAGGATTCAGAAAGCCCAGGGCATGCCTGAAG
GAGGGGGCGCTCTCGCAGAAGGCTTTCAAGTGAGGATGTTTGAGTT
TCAGAATTTTGAGAGGAGATTTGAGGAGTGCATCTCCCAGTCTGCA
GTGAAGACCAAGTTTGAGCAGCACACGGTCCGGGCCAAGCAGATTG
CAGAGGCGGTTCGACTCATCATGGACTCCCTGCACATGGCGGCTCG
GGAGCAGCAGGTTTACTGCGAGGAAATGCGTGAAGAGCGGCAAGAC
CGACTGAAATTTATTGACAAACAGCTGGAGCTCTTGGCTCAAGACT
ATAAGCTGCGAATTAAGCAGATTACGGAGGAAGTGGAGAGGCAGGT
GTCGACTGCAATGGCCGAGGAGATCAGGCGCCTCTCTGTACTGGTG
GACGATTACCAGATGGACTTCCACCCTTCTCCAGTAGTCCTCAAGG
TTTATAAGAATGAGCTGCACCGCCACATAGAGGAAGGACTGGGTCG
AAACATGTCTGACCGCTGCTCCACGGCCATCACCAACTCCCTGCAG
ACCATGCAGCAGGACATGATAGATGGCTTGAAACCCCTCCTTCCTG
TGTCTGTGCGGAGTCAGATAGACATGCTGGTCCCACGCCAGTGCTT
CTCCCTCAACTATGACCTAAACTGTGACAAGCTGTGTGCTGACTTC
CAGGAAGACATTGAGTTCCATTTCTCTCTCGGATGGACCATGCTGG
TGAATAGGTTCCTGGGCCCCAAGAACAGCCGTCGGGCCTTGATGGG
CTACAATGACCAGGTCCAGCGTCCCATCCCTCTGACGCCAGCCAAC
CCCAGCATGCCCCCACTGCCACAGGGCTCGCTCACCCAGGAGGAGT
TCATGGTTTCCATGGTTACCGGCCTGGCCTCCTTGACATCCAGGAC
CTCCATGGGCATTCTTGTTGTTGGAGGAGTGGTGTGGAAGGCAGTG
GGCTGGCGGCTCATTGCCCTCTCCTTTGGGCTCTATGGCCTCCTCT
ACGTCTATGAGCGTCTGACCTGGACCACCAAGGCCAAGGAGAGGGC
CTTCAAGCGCCAGTTTGTGGAGCATGCCAGCGAGAAGCTGCAGCTT
GTCATCAGCTACACTGGCTCCAACTGCAGCCACCAAGTCCAGCAGG
AACTGTCTGGGACCTTTGCTCATCTGTGTCAGCAAGTTGACGTCAC
CCGGGAGAACCTGGAGCAGGAAATTGCCGCCATGAACAAGAAAATT
GAGGTTCTTGACTCACTTCAGAGCAAAGCAAAGCTGCTCAGGAATA
AAGCCGGTTGGTTGGACAGTGAGCTCAACATGTTCACACACCAGTA
CCTGCAGCCCAGCAGATAGTGGGCACCTGAGGCGGAGTCTGCGTGG
AGAGGGGCGGTGCTGCCAGCCCTAAGTGCCATGTGGGCTCCCCCAG
GGGCACGTGTGGCTCCTGCCCCCTGGCCACTGCCAAGAGAATGAAG
CACCCAGTCTCGTACCATTTTGAGCCCTCCAGCACTACTTATTTTC
CCCCACCTTTGCCTGCTGTTGCTGGAAGAGCTGGCTCATACCCCCA
AAGGACACTTTCAGCGACAGCTATGGACAGCATGGTACCAAGGAGT
TAAGTTGAGGCTTTTTCCAGCTTTCTCTGGTTCATTTGATTGCTTG
ATAAGGCCTCAGGATCTCAGCATTGCACAATGCCTCATGGAAGCCT
TTGAGGGTATCACACAGACACCCCCACCTTCCTCCAGCCTGTGCGC
ACCTGCCCTCCTTGCAGCCCAGCACACCTGCAGGTGTAAGGGACGA
TTGGAGTTTCTTCCCAGAGAGTCTGTCCCAGAAGGACTGTGGCTTG
TGTGTGTCCATCTCGCCTGTTGGCTCAGTGCTTCATCCCATTTGCA
GAGCCTCAGACACGTCTTGGTGGTGAGGCTCAGTTACCCCTGGGCT
TAGGCTGAGGCGGGCCCTGTGCTGGGGGTGGTAGAAAGGATGCTGC
TGAGGCAGCTGGAGGAGTGGGAGTAGCTCAGAGGGGAGGGCTGTTG
GATGTATGGGGAGCTGGCAGAGCAGGTGGCAGTCACTGGGACAAGG
AGGGACTTGCCTCTCTTCTCATTATTGTGTCCTTTGCTTTAGTGTC
AGTCCTGGACTTGTGCAGGCCTGTTTTGTGTAGATCTGTTTTGGAA
GATGGCATGGTCTAGGTGGTTGAAGGATGTAGTAGAAGGATGGATG
GTGGAAGGTGGGGACGTTGGTGGCTGGCTGAGGTGCATGGGCCCCA
CACAGGACAGCTGGAGAATGGGCCGTCCACTTGGCCTCGTTCTGCG
AGGGGCTCATGGGTCTGAGAGCCCCCACCCACTAGGCTTGATTGCA
TCCCTGTTGTGCCCTTTAAGAGACATGTTTCCACCCCACCCCCAAC
CTTGTCCCAAGTGCCCTGGACTAAATTTCCTGTGCCAGTGACTGCA
GTTGGCCAAGGGACAATGTGGAAAACCCAGTGTCCATCTTTCCACC
CTCCCTGATCTCCAGAACCTTCGACTGACCCCCTTGTCTTTATGCT
GATGTTGAGTTTTGGGATTGTTACTGGTTGAAGTGGGGGCAGATGC
CTGTCACCAAGGTGTTGACTGTGTGAGAAAAGCAGTTTGGGTGACA
AATCCTGTGTGGCACAAGTTGGATCGCTTCCTAGAAATAAGCAACA
CCTCTCCCAAAAAGCAGCCCACAAGGCAGGGGCCCAGCAGCCCAGC
CATCACTCATCTTTGAGGAAATGAGTTGGTAGCCTCTGTGCACTGT
TTGGTGGCCACATCACAGGTGATGTCCTGTTCACATACCTGCTTGT
ATTTAAAGCCCTCAGTCTGTCCTGTTGTGTGGGGCGAAGTGATGGA
CTCTGCCAGGTGGACATGCTGTGGGTGGATGTTCCCGGCGTGTGCC
GGGCCTGAATGGACAGGGGCCACTTCACAGCATGTCAGGGAAAATC
ACTGTCACACAATTCCAATGGATTTTGTGCTCTTTTTGAAAAAAAA
AAATTCTTTAGCGTAAACATGAATTTTTTTTCAATGTAGCCCCTGG
GGAATGAATGAAATTTTGAGCTTCTTCAATACGTAAAATTAAATTT
ATACCACTGAGGGAGAGACCCTTTCTGAAAGAAGTATGGCCAAAAG
CACTTTAATGCTGCTGACATTGTTGTTTTTATGTTCATTTGCTGGA
GCGCAAGACGTGCTGACACAGTGAGTTTTCTCTGATGTATTTAAGG
TGATGTATTTGCTTGAGTTACTCCTGTATCATTGCTCATAATATTG
GAAACTAAAATAAAACCTAGTTGGAAATCC
Mfn 2 nucleic acid sequence NM_014874
(SEQ ID NO: 8)
GTAGTGAGTGTGATGGCCGCCGCGAGGCCGGGAAGGTGAAGTCAGG
ACTGGTGGAGTCAACACAGTCAATCAATAGCCAACCTCAACCTGAG
ACAGGACAGAAGAGAACTCAGAATCTTTTTGTCTTTTGGACTTCAG
CCATGTCCATGATGCCTACCCTGTGAAGATCTCTCACCATCCAAAA
AACGCAATGTCCCTGCTCTTCTCTCGATGCAACTCTATCGTCACAG
TCAAGAAAAATAAGAGACACATGGCTGAGGTGAATGCATCCCCACT
TAAGCACTTTGTCACTGCCAAGAAGAAGATCAATGGCATTTTTGAG
CAGCTGGGGGCCTACATCCAGGAGAGCGCCACCTTCCTTGAAGACA
CGTACAGGAATGCAGAACTGGACCCCGTTACCACAGAAGAACAGGT
TCTGGACGTCAAAGGTTACCTATCCAAAGTGAGAGGCATCAGTGAG
GTGCTGGCTCGGAGGCACATGAAAGTGGCTTTTTTTGGCCGGACGA
GCAATGGGAAGAGCACCGTGATCAATGCCATGCTCTGGGACAAAGT
TCTGCCCTCTGGGATTGGCCACACCACCAATTGCTTCCTGCGGGTA
GAGGGCACAGATGGCCATGAGGCCTTTCTCCTTACCGAGGGCTCAG
AGGAAAAGAGGAGTGCCAAGACTGTGAACCAGCTGGCCCATGCCCT
CCACCAGGACAAGCAGCTCCATGCCGGCAGCCTAGTGAGTGTGATG
TGGCCCAACTCTAAGTGCCCACTTCTGAAGGATGACCTCGTTTTGA
TGGACAGCCCTGGTATTGATGTCACCACAGAGCTGGACAGCTGGAT
TGACAAGTTTTGTCTGGATGCTGATGTGTTTGTGCTGGTGGCCAAC
TCAGAGTCCACCCTGATGCAGACGGAAAAGCACTTCTTCCACAAGG
TGAGTGAGCGTCTCTCCCGGCCAAACATCTTCATCCTGAACAACCG
CTGGGATGCATCTGCCTCAGAGCCCGAGTACATGGAGGAGGTGCGG
CGGCAGCACATGGAGCGTTGTACCAGCTTCCTGGTGGATGAGCTGG
GCGTGGTGGATCGATCCCAGGCCGGGGACCGCATCTTCTTTGTGTC
TGCTAAGGAGGTGCTCAACGCCAGGATTCAGAAAGCCCAGGGCATG
CCTGAAGGAGGGGGCGCTCTCGCAGAAGGCTTTCAAGTGAGGATGT
TTGAGTTTCAGAATTTTGAGAGGAGATTTGAGGAGTGCATCTCCCA
GTCTGCAGTGAAGACCAAGTTTGAGCAGCACACGGTCCGGGCCAAG
CAGATTGCAGAGGCGGTTCGACTCATCATGGACTCCCTGCACATGG
CGGCTCGGGAGCAGCAGGTTTACTGCGAGGAAATGCGTGAAGAGCG
GCAAGACCGACTGAAATTTATTGACAAACAGCTGGAGCTCTTGGCT
CAAGACTATAAGCTGCGAATTAAGCAGATTACGGAGGAAGTGGAGA
GGCAGGTGTCGACTGCAATGGCCGAGGAGATCAGGCGCCTCTCTGT
ACTGGTGGACGATTACCAGATGGACTTCCACCCTTCTCCAGTAGTC
CTCAAGGTTTATAAGAATGAGCTGCACCGCCACATAGAGGAAGGAC
TGGGTCGAAACATGTCTGACCGCTGCTCCACGGCCATCACCAACTC
CCTGCAGACCATGCAGCAGGACATGATAGATGGCTTGAAACCCCTC
CTTCCTGTGTCTGTGCGGAGTCAGATAGACATGCTGGTCCCACGCC
AGTGCTTCTCCCTCAACTATGACCTAAACTGTGACAAGCTGTGTGC
TGACTTCCAGGAAGACATTGAGTTCCATTTCTCTCTCGGATGGACC
ATGCTGGTGAATAGGTTCCTGGGCCCCAAGAACAGCCGTCGGGCCT
TGATGGGCTACAATGACCAGGTCCAGCGTCCCATCCCTCTGACGCC
AGCCAACCCCAGCATGCCCCCACTGCCACAGGGCTCGCTCACCCAG
GAGGAGTTCATGGTTTCCATGGTTACCGGCCTGGCCTCCTTGACAT
CCAGGACCTCCATGGGCATTCTTGTTGTTGGAGGAGTGGTGTGGAA
GGCAGTGGGCTGGCGGCTCATTGCCCTCTCCTTTGGGCTCTATGGC
CTCCTCTACGTCTATGAGCGTCTGACCTGGACCACCAAGGCCAAGG
AGAGGGCCTTCAAGCGCCAGTTTGTGGAGCATGCCAGCGAGAAGCT
GCAGCTTGTCATCAGCTACACTGGCTCCAACTGCAGCCACCAAGTC
CAGCAGGAACTGTCTGGGACCTTTGCTCATCTGTGTCAGCAAGTTG
ACGTCACCCGGGAGAACCTGGAGCAGGAAATTGCCGCCATGAACAA
GAAAATTGAGGTTCTTGACTCACTTCAGAGCAAAGCAAAGCTGCTC
AGGAATAAAGCCGGTTGGTTGGACAGTGAGCTCAACATGTTCACAC
ACCAGTACCTGCAGCCCAGCAGATAGTGGGCACCTGAGGCGGAGTC
TGCGTGGAGAGGGGCGGTGCTGCCAGCCCTAAGTGCCATGTGGGCT
CCCCCAGGGGCACGTGTGGCTCCTGCCCCCTGGCCACTGCCAAGAG
AATGAAGCACCCAGTCTCGTACCATTTTGAGCCCTCCAGCACTACT
TATTTTCCCCCACCTTTGCCTGCTGTTGCTGGAAGAGCTGGCTCAT
ACCCCCAAAGGACACTTTCAGCGACAGCTATGGACAGCATGGTACC
AAGGAGTTAAGTTGAGGCTTTTTCCAGCTTTCTCTGGTTCATTTGA
TTGCTTGATAAGGCCTCAGGATCTCAGCATTGCACAATGCCTCATG
GAAGCCTTTGAGGGTATCACACAGACACCCCCACCTTCCTCCAGCC
TGTGCGCACCTGCCCTCCTTGCAGCCCAGCACACCTGCAGGTGTAA
GGGACGATTGGAGTTTCTTCCCAGAGAGTCTGTCCCAGAAGGACTG
TGGCTTGTGTGTGTCCATCTCGCCTGTTGGCTCAGTGCTTCATCCC
ATTTGCAGAGCCTCAGACACGTCTTGGTGGTGAGGCTCAGTTACCC
CTGGGCTTAGGCTGAGGCGGGCCCTGTGCTGGGGGTGGTAGAAAGG
ATGCTGCTGAGGCAGCTGGAGGAGTGGGAGTAGCTCAGAGGGGAGG
GCTGTTGGATGTATGGGGAGCTGGCAGAGCAGGTGGCAGTCACTGG
GACAAGGAGGGACTTGCCTCTCTTCTCATTATTGTGTCCTTTGCTT
TAGTGTCAGTCCTGGACTTGTGCAGGCCTGTTTTGTGTAGATCTGT
TTTGGAAGATGGCATGGTCTAGGTGGTTGAAGGATGTAGTAGAAGG
ATGGATGGTGGAAGGTGGGGACGTTGGTGGCTGGCTGAGGTGCATG
GGCCCCACACAGGACAGCTGGAGAATGGGCCGTCCACTTGGCCTCG
TTCTGCGAGGGGCTCATGGGTCTGAGAGCCCCCACCCACTAGGCTT
GATTGCATCCCTGTTGTGCCCTTTAAGAGACATGTTTCCACCCCAC
CCCCAACCTTGTCCCAAGTGCCCTGGACTAAATTTCCTGTGCCAGT
GACTGCAGTTGGCCAAGGGACAATGTGGAAAACCCAGTGTCCATCT
TTCCACCCTCCCTGATCTCCAGAACCTTCGACTGACCCCCTTGTCT
TTATGCTGATGTTGAGTTTTGGGATTGTTACTGGTTGAAGTGGGGG
CAGATGCCTGTCACCAAGGTGTTGACTGTGTGAGAAAAGCAGTTTG
GGTGACAAATCCTGTGTGGCACAAGTTGGATCGCTTCCTAGAAATA
AGCAACACCTCTCCCAAAAAGCAGCCCACAAGGCAGGGGCCCAGCA
GCCCAGCCATCACTCATCTTTGAGGAAATGAGTTGGTAGCCTCTGT
GCACTGTTTGGTGGCCACATCACAGGTGATGTCCTGTTCACATACC
TGCTTGTATTTAAAGCCCTCAGTCTGTCCTGTTGTGTGGGGCGAAG
TGATGGACTCTGCCAGGTGGACATGCTGTGGGTGGATGTTCCCGGC
GTGTGCCGGGCCTGAATGGACAGGGGCCACTTCACAGCATGTCAGG
GAAAATCACTGTCACACAATTCCAATGGATTTTGTGCTCTTTTTGA
AAAAAAAAAATTCTTTAGCGTAAACATGAATTTTTTTTCAATGTAG
CCCCTGGGGAATGAATGAAATTTTGAGCTTCTTCAATACGTAAAAT
TAAATTTATACCACTGAGGGAGAGACCCTTTCTGAAAGAAGTATGG
CCAAAAGCACTTTAATGCTGCTGACATTGTTGTTTTTATGTTCATT
TGCTGGAGCGCAAGACGTGCTGACACAGTGAGTTTTCTCTGATGTA
TTTAAGGTGATGTATTTGCTTGAGTTACTCCTGTATCATTGCTCAT
AATATTGGAAACTAAAATAAAACCTAGTTGGAAATCC
Mfn 2 amino acid sequence NP_001121132
(SEQ ID NO: 9)
MSLLFSRCNSIVTVKKNKRHMAEVNASPLKHFVTAKKKINGIFEQL
GAYIQESATFLEDTYRNAELDPVTTEEQVLDVKGYLSKVRGISEVL
ARRHMKVAFFGRTSNGKSTVINAMLWDKVLPSGIGHTTNCFLRVEG
TDGHEAFLLTEGSEEKRSAKTVNQLAHALHQDKQLHAGSLVSVMWP
NSKCPLLKDDLVLMDSPGIDVTTELDSWIDKFCLDADVFVLVANSE
STLMQTEKHFFHKVSERLSRPNIFILNNRWDASASEPEYMEEVRRQ
HMERCTSFLVDELGVVDRSQAGDRIFFVSAKEVLNARIQKAQGMPE
GGGALAEGFQVRMFEFQNFERRFEECISQSAVKTKFEQHTVRAKQI
AEAVRLIMDSLHMAAREQQVYCEEMREERQDRLKFIDKQLELLAQD
YKLRIKQITEEVERQVSTAMAEEIRRLSVLVDDYQMDFHPSPVVLK
VYKNELHRHIEEGLGRNMSDRCSTAITNSLQTMQQDMIDGLKPLLP
VSVRSQIDMLVPRQCFSLNYDLNCDKLCADFQEDIEFHFSLGWTML
VNRFLGPKNSRRALMGYNDQVQRPIPLTPANPSMPPLPQGSLTQEE
FMVSMVTGLASLTSRTSMGILVVGGVVWKAVGWRLIALSFGLYGLL
YVYERLTWTTKAKERAFKRQFVEHASEKLQLVISYTGSNCSHQVQQ
ELSGTFAHLCQQVDVTRENLEQEIAAMNKKIEVLDSLQSKAKLLRN
KAGWLDSELNMFTHQYLQPSR
Mfn2 amino acid sequence NP_055689
(SEQ ID NO: 10)
MSLLFSRCNSIVTVKKNKRHMAEVNASPLKHFVTAKKKINGIFEQL
GAYIQESATFLEDTYRNAELDPVTTEEQVLDVKGYLSKVRGISEVL
ARRHMKVAFFGRTSNGKSTVINAMLWDKVLPSGIGHTTNCFLRVEG
TDGHEAFLLTEGSEEKRSAKTVNQLAHALHQDKQLHAGSLVSVMWP
NSKCPLLKDDLVLMDSPGIDVTTELDSWIDKFCLDADVFVLVANSE
STLMQTEKHFFHKVSERLSRPNIFILNNRWDASASEPEYMEEVRRQ
HMERCTSFLVDELGVVDRSQAGDRIFFVSAKEVLNARIQKAQGMPE
GGGALAEGFQVRMFEFQNFERRFEECISQSAVKTKFEQHTVRAKQI
AEAVRLIMDSLHMAAREQQVYCEEMREERQDRLKFIDKQLELLAQD
YKLRIKQITEEVERQVSTAMAEEIRRLSVLVDDYQMDFHPSPVVLK
VYKNELHRHIEEGLGRNMSDRCSTAITNSLQTMQQDMIDGLKPLLP
VSVRSQIDMLVPRQCFSLNYDLNCDKLCADFQEDIEFHFSLGWTML
VNRFLGPKNSRRALMGYNDQVQRPIPLTPANPSMPPLPQGSLTQEE
FMVSMVTGLASLTSRTSMGILVVGGVVWKAVGWRLIALSFGLYGLL
YVYERLTWTTKAKERAFKRQFVEHASEKLQLVISYTGSNCSHQVQQ
ELSGTFAHLCQQVDVTRENLEQEIAAMNKKIEVLDSLQSKAKLLRN
KAGWLDSELNMFTHQYLQPSR
Human Mfn2 siRNA
(SEQ ID NO: 11)
GACUAUAAGCUGCGAAUUACAUGAGGCCUUUCUCCUUAGCAACUCUA
UCGUCACAGUGGUGGACGAUUACCAGAUG
Human Mfn1 siRNA
(SEQ ID NO: 12)
CGAUGAAGUAAACGCCUUACAUGAUAGGAGGAAACGAACAGAAUAU
AUGGAAGACGUGGAAGUUCUUAGUGCUAGA
sgRNA1
(SEQ ID NO: 13)
TGTTGTTGTGACGATTGCTC
sgRNA2
(SEQ ID NO: 14)
TTACGGGACTAGAAAATCAG
sgRNA3
(SEQ ID NO: 15)
TGCTGTGACATTTAAGCGGT
sgRNA4
(SEQ ID NO: 16)
CGAATGCTGTGACATTTAAG
sgRNA5
(SEQ ID NO: 17)
TTTATAAAATGCAATAGGT.

1. A method for treating a disease associated with mitochondrial dysfunction in a subject in need thereof, the method comprising administering to the subject a therapeutically effective amount of a mitofusin (Mfn) modulator.

2. The method of claim 1, wherein the Mfn modulator increases or inhibits expression or activity of a mitofusin.

3. (canceled)

4. The method of claim 1, wherein the mitofusin is mitofusin 2 (Mfn2).

5. The method of claim 1, wherein the Mfn modulator directly or indirectly modulates expression or activity of a mitofusin.

6. The method of claim 5, wherein the Mfn modulator selectively inhibits expression or activity of a mitofusin.

7. (canceled)

8. The method of claim 1, wherein the Mfn modulator is a nucleic acid, polypeptide, or small molecule.

9. The method of claim 8, wherein the nucleic acid is an interfering nucleic acid selected from the group consisting of double stranded RNA (dsRNA), siRNA, shRNA, miRNA, and antisense oligonucleotide (ASO).

10. The method of claim 8, wherein the polypeptide comprises a CRISPR-Cas nuclease.

11. The method of claim 8, wherein the small molecule comprises a proteolysis targeting chimera (PROTAC), a kinase modulator, or an E3 ubiquitin ligase modulator.

12. The method of claim 11, wherein the kinase modulator comprises a PINK1 modulator, or wherein the E3 ubiquitin ligase modulator comprises a Mule ligase modulator or Parkin modulator

13. (canceled)

14. The method of claim 1, wherein the subject is a mammal.

15. The method of claim 14, wherein the mammal is a human.

16. The method of claim 1, wherein the subject comprises one or more mutations in vps13d, wherein the one or more mutations comprise a frameshift, missense, or partial duplication mutation.

17. (canceled)

18. The method of claim 1, wherein the disease associated with mitochondrial dysfunction is a neurological movement disorder.

19. The method of claim 1, wherein the Mfn modulator is delivered to a neuron of the subject.

20. The method of claim 1, wherein the therapeutically effective amount restores normal mitochondrial function in the subject.

21. A method of treating a subject having a VPS13D-associated disease, the method comprising identifying the subject as having the VPS13D-associated disease by detecting in a biological sample obtained from a subject an increased level of mitofusin (Mfn) expression or activity relative to a control sample, and administering to the subject one or more Mfn modulators.

22. The method of claim 21, wherein the subject is a mammal.

23. The method of claim 21, wherein the mitofusin is Mitofusin 2 (Mfn2).

24. The method of claim 21, wherein the subject comprises one or more mutations in vps13d.

25. (canceled)
